SEQUENCE LISTING (59 sequences)

SEQ ID NO: 1 - protein, 12 aa, homo sapiens
  spacer (IgG4hinge)
ESKYGPPCPPCP

SEQ ID NO: 2 - DNA, 36 bp, homo sapiens
  spacer (IgG4hinge)
gaatctaagtacggaccgccctgccccccttgccct

SEQ ID NO: 3 - protein, 119 aa, homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A
LEGGGEGRGSLLTCGDVEENPGPR

SEQ ID NO: 7 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, homo sapiens
  CD28 (amino acids 153-179 of Accession No. P10747)
FWVLVVVGGVLACYSLLVTVAFIIFWV

SEQ ID NO: 9 - protein, 66 aa, homo sapiens
  CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 10 - protein, 41 aa, homo sapiens
  CD28 (amino acids 180-220 of P10747)
RSKRSRLLHSDYMNMTPRRPGPTRKHYQPYAPPRDFAAYRS

SEQ ID NO: 11 - protein, 41 aa, homo sapiens
  CD28 (LL to GG)
RSKRSRGGHSDYMNMTPRRPGPTRKHYQPYAPPRDFAAYRS

SEQ ID NO: 12 - protein, 42 aa, homo sapiens
  4-1BB (amino acids 214-255 of Q07011.1)
KRGRKKLLYIFKQPFMRPVQTTQEEDGCSCRFPEEEEGGCEL

SEQ ID NO: 13 - protein, 112 aa, homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - protein, 18 aa, Artificial Sequence
  T2A
EGRGSLLTCGDVEENPGP

SEQ ID NO: 18 - protein, 22 aa, Artificial Sequence
  P2A
GSGATNFSLLKQAGDVEENPGP

SEQ ID NO: 19 - protein, 19 aa, Artificial Sequence
  P2A
ATNFSLLKQAGDVEENPGP

SEQ ID NO: 20 - protein, 20 aa, Artificial Sequence
  E2A
QCTNYALLKLAGDVESNPGP

SEQ ID NO: 21 - protein, 22 aa, Artificial Sequence
  F2A
VKQTLNFDLLKLAGDVESNPGP

SEQ ID NO: 22 - protein, 10 aa, Artificial Sequence
  linker
  REPEAT (5)...(9): SGGGG is repeated 5 times
PGGGSGGGGP

SEQ ID NO: 23 - protein, 17 aa, Artificial Sequence
  linker
GSADDAKKDAAKKDGKS

SEQ ID NO: 24 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 25 - protein, 22 aa, Artificial Sequence
  GMCSFR alpha chain signal sequence
MLLLVTSLLLCELPHPAFLLIP

SEQ ID NO: 26 - protein, 18 aa, Artificial Sequence
  CD8 alpha signal peptide
MALPVTALLLPLALLLHA

SEQ ID NO: 27 - protein, 15 aa, Artificial Sequence
  Hinge
EPKSCDKTHTCPPCP

SEQ ID NO: 28 - protein, 12 aa, Artificial Sequence
  Hinge
ERKCCVECPPCP

SEQ ID NO: 29 - protein, 61 aa, Artificial Sequence
  Hinge
ELKTPLGDTHTCPRCPEPKSCDTPPPCPRCPEPKSCDTPPPCPRCPEPKSCDTPPPCPRCP

SEQ ID NO: 30 - protein, 12 aa, Artificial Sequence
  Hinge
ESKYGPPCPSCP

SEQ ID NO: 31 - protein, 12 aa, Artificial Sequence
  Hinge
ESKYGPPCPPCP

SEQ ID NO: 32 - protein, 9 aa, Artificial Sequence
  Hinge
YGPPCPPCP

SEQ ID NO: 33 - protein, 10 aa, Artificial Sequence
  Hinge
KYGPPCPPCP

SEQ ID NO: 34 - protein, 14 aa, Artificial Sequence
  Hinge
EVVVKYGPPCPPCP

SEQ ID NO: 35 - protein, 11 aa, Artificial Sequence
  CDR L1
RASQDISKYLN

SEQ ID NO: 36 - protein, 7 aa, Artificial Sequence
  CDR L2
SRLHSGV

SEQ ID NO: 37 - protein, 9 aa, Artificial Sequence
  CDR L3
GNTLPYTFG

SEQ ID NO: 38 - protein, 5 aa, Artificial Sequence
  CDR H1
DYGVS

SEQ ID NO: 39 - protein, 16 aa, Artificial Sequence
  CDR H2
VIWGSETTYYNSALKS

SEQ ID NO: 40 - protein, 7 aa, Artificial Sequence
  CDR H3
YAMDYWG

SEQ ID NO: 41 - protein, 120 aa, Artificial Sequence
  VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 107 aa, Artificial Sequence
  VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 43 - protein, 245 aa, Artificial Sequence
  scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 11 aa, Artificial Sequence
  CDR L1
KASQNVGTNVA

SEQ ID NO: 45 - protein, 7 aa, Artificial Sequence
  CDR L2
SATYRNS

SEQ ID NO: 46 - protein, 9 aa, Artificial Sequence
  CDR L3
QQYNRYPYT

SEQ ID NO: 47 - protein, 5 aa, Artificial Sequence
  CDR H1
SYWMN

SEQ ID NO: 48 - protein, 17 aa, Artificial Sequence
  CDR H2
QIYPGDGDTNYNGKFKG

SEQ ID NO: 49 - protein, 13 aa, Artificial Sequence
  CDR H3
KTISSVVDFYFDY

SEQ ID NO: 50 - protein, 122 aa, Artificial Sequence
  VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 51 - protein, 108 aa, Artificial Sequence
  VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 52 - protein, 15 aa, Artificial Sequence
  linker
GGGGSGGGGSGGGGS

SEQ ID NO: 53 - protein, 245 aa, Artificial Sequence
  scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 54 - protein, 12 aa, Artificial Sequence
  HC-CDR3
HYYYGGSYAMDY

SEQ ID NO: 55 - protein, 7 aa, Artificial Sequence
  LC-CDR2
HTSRLHS

SEQ ID NO: 56 - protein, 9 aa, Artificial Sequence
  LC-CDR3
QQGNTLPYT

SEQ ID NO: 57 - DNA, 735 bp, Artificial Sequence
  Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 58 - protein, 5 aa, Artificial Sequence
  Hinge
  VARIANT (1)...(1): Xaa is glycine, cysteine or arginine
  VARIANT (4)...(4): Xaa is cysteine or threonine
XPPXP

SEQ ID NO: 59 - protein, 18 aa, Artificial Sequence
  Linker
GSTSGSGKPGSGEGSTKG

(19) United States
(12) Patent Application Publication
(10) Pub. No.: US 2023/0071910 A1
(43) Pub. Date: Mar. 9, 2023

(54) METHODS FOR DOSING AND TREATMENT OF FOLLICULAR LYMPHOMA AND MARGINAL ZONE LYMPHOMA IN ADOPTIVE CELL THERAPY

(71) Applicant: Juno Therapeutics, Inc., Seattle, WA (US)

(72) Inventor: Thalia FARAZI, Seattle, WA (US)

(73) Assignee: Juno Therapeutics, Inc., Seattle, WA (US)

(21) Appl. No.: 17/794,245

(22) PCT Filed: Jan. 22, 2021
(86) PCT No.: PCT/US2021/014775

§ 371 Date: Jul. 20, 2022

Related U.S. Application Data
(60) Provisional application No. 63/068,975, filed on Aug. 21, 2020.
(60) Provisional application No. 63/037,542, filed on Jun. 10, 2020.
(60) Provisional application No. 62/965,774, filed on Jan. 24, 2020.

(51) Int. Cl. A61K 35/17; C07K 14/725; C07K 16/28; C12N 5/0783

(52) U.S. Cl. CPC A61K 35/17; C07K 14/7051; C07K 16/2803; C12N 5/0636; A61K 2039/5156

(57) ABSTRACT

Provided herein are methods of administering a dose of T cells for treating subjects with indolent non-Hodgkin3 s lymphoma (NHL), and related methods, compositions, uses and articles of manufacture. The cells express a recombinant receptor such as a chimeric antigen receptor (CAR) for targeting an antigen of the lymphoma, such as CD19. In some embodiments, the methods are for treating grade 1-3 A follicular lymphoma (FL 1-3 A) or marginal zone lymphoma (MZL), including in heavily pretreated or poor-prognosis subjects, such as subjects that have relapsed after treatment with, or are refractory to treatment with, one or more prior therapies.

### CROSS-REFERENCE TO RELATED APPLICATIONS

This application claims priority to U.S. provisional application No. 62/965,774, filed Jan. 24, 2020, entitled “METHODS FOR DOSING AND TREATMENT OF FOLLICULAR LYMPHOMA AND MARGINAL ZONE LYMPHOMA IN ADOPTIVE CELL THERAPY,” U.S. provisional application No. 63/037,542, filed Jun. 10, 2020, entitled “METHODS FOR DOSING AND TREATMENT OF FOLLICULAR LYMPHOMA AND MARGINAL ZONE LYMPHOMA IN ADOPTIVE CELL THERAPY,” and U.S. provisional application No. 63/068,975, filed Aug. 21, 2020, entitled “METHODS FOR DOSING AND TREATMENT OF FOLLICULAR LYMPHOMA AND MARGINAL ZONE LYMPHOMA IN ADOPTIVE CELL THERAPY,” the contents of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 735042023440SeqList.TXT, created Jan. 13, 2021, which is 35,356 bytes in size. The information in the electronic format of the Sequence Listing is incorporated by reference in its entirety.

### FIELD

The present disclosure relates in some aspects to adoptive cell therapy involving the administration of doses of cells for treating subjects with indolent non-Hodgkin's lymphoma (NHL), including high-risk subjects, and related methods, compositions, uses and articles of manufacture. The cells generally express recombinant receptors such as chimeric antigen receptors (CARs) for targeting an antigen, such as CD19, on cells of the lymphoma. In some embodiments, the indolent lymphoma is a grade 1-3A follicular lymphoma (FL 1-3A), such as relapsed or refractory FL. In some embodiments, the disease or condition is a marginal zone lymphoma (MZL), such as a relapsed or refractory MZL. In some embodiments, the subject is of a specific group or subset of FL 1-3A or MZL subjects, such as heavily pretreated or poor-prognosis subjects.

### BACKGROUND

Indolent non-Hodgkin lymphomas (NHL) subtypes are a slow-growing lymphoma disease that are commonly treated with chemoimmunotherapeutics, such as anti-CD20 targeted therapies or alkylating agents. Follicular lymphoma is the most common subtype, accounting for 10% to 20% of all lymphoma cases. Marginal zone lymphoma accounts for about 8% to 12% of all B cell NHL cases. Many patients eventually relapse or become refractory to available therapies, and second-line, third-line, and particularly fourth-line treatments are limited. Effective therapies for patients with FL 1-3A and MZL who have failed one or more prior therapy are needed. Provided are methods and uses that meet such needs.

### SUMMARY

Provided herein are methods of treating a subject having or suspected of having a disease or condition that is follicular lymphoma(FL) Grade 1, 2 or 3A, the method including administering to the subject a dose of CD4⁺ and CD8⁺ T cells, wherein T cells of the dose comprises a chimeric antigen receptor (CAR) that specifically binds to CD19, wherein: the subject has relapsed or is refractory to treatment after at least one prior line of therapy for treating FL grade 1, 2 or 3A, wherein at least one of the at least one prior line of therapy includes treatment with an anti-CD20 antibody and an alkylating agent; the dose of T cells comprises between at or about 5×10⁷ CAR-expressing T cells and at or about 1.5×10⁸ CAR-expressing T cells, inclusive; the dose of T cells comprises a ratio of approximately 1:1 CD4+ T cells expressing the CAR to CD8+ T cells expressing the CAR; and the administration comprises administering a plurality of separate compositions, wherein the plurality of separate compositions comprises a first composition comprising the CD8+ CAR-expressing T cells and a second composition comprising the CD4+ CAR-expressing T cells.

Also provided herein is use of a dose of CD4⁺ and CD8⁺ T cells to treat a subject having or suspected of having a disease that is relapsed/refractory (r/r) follicular lymphoma (FL) Grade 1, 2 or 3A, wherein: T cells of the dose include a chimeric antigen receptor (CAR) that specifically binds to CD19; the subject has relapsed or is refractory to treatment after at least one prior line of therapy for treating FL Grade 1, 2 or 3A, wherein at least one of the at least one prior line of therapy includes treatment with an anti-CD20 antibody and an alkylating agent; the dose of T cells contains between at or about 5×10⁷ CAR-expressing T cells and at or about 1.5×10⁸ CAR-expressing T cells, inclusive; the dose of T cells has a ratio of approximately 1:1 CD4⁺ T cells expressing the CAR to CD8⁺ T cells expressing the CAR; and the dose is formulated for administration as a plurality of separate compositions, wherein the plurality of separate compositions includes a first composition containing the CD8⁺ CAR-expressing T cells and a second composition containing the CD4⁺ CAR-expressing T cells.

Also provided herein is use of a dose of CD4⁺ and CD8⁺ T cells in the manufacture of a medicament for the treatment of a subject having or suspected of having a disease that is relapsed/refractory (r/r) follicular lymphoma (FL) Grade 1, 2 or 3A, wherein: T cells of the dose include a chimeric antigen receptor (CAR) that specifically binds to CD19; the subject has relapsed or is refractory to treatment after at least one prior line of therapy for treating FL Grade 1, 2 or 3A, wherein at least one of the at least one prior line of therapy includes treatment with an anti-CD20 antibody and an alkylating agent; the dose of T cells contains between at or about 5×10⁷ CAR-expressing T cells and at or about 1.5×10⁸ CAR-expressing T cells, inclusive; the dose of T cells has a ratio of approximately 1:1 CD4⁺ T cells expressing the CAR to CD8⁺ T cells expressing the CAR; and the dose is formulated for administration as a plurality of separate compositions, wherein the plurality of separate compositions includes a first composition containing the CD8⁺ CAR-expressing T cells and a second composition containing the CD4⁺ CAR-expressing T cells.

In some embodiments, the at least one prior line of therapy is one prior line of therapy.

In some of any of the provided embodiments, the subject had progression of the disease within 24 months of diagnosis after completing the one prior line of therapy (POD24). In some of any of the provided embodiments, the subject had progression of the disease within 24 months of initiation of the one prior line of therapy after completing the one prior line of therapy (POD24). In some of any of the provided embodiments, the subject had progression of the disease within 24 months of diagnosis after completing a chemoimmunotherapeutic combination therapy for treating the disease (POD24). In some of any of the provided embodiments, the subject had progression of the disease within 24 months of initiation of treatment after completing a chemoimmunotherapeutic combination therapy for treating the disease (POD24). In some of any such embodiments, the chemoimmunotherapeutic combination therapy includes rituximab, cyclophosphamide, vincristine, doxorubicin, and prednisolone (R-CHOP). In some of any of the provided embodiments, the subject had progression of the disease within 24 months of diagnosis after completing treatment with R-CHOP (POD24). In some of any of the provided embodiments, the subject had progression of the disease within 24 months of initiation of treatment with R-CHOP (POD24).

In some of any such embodiments, the chemoimmunotherapeutic combination therapy includes an anti-CD20 monoclonal antibody and an alkylating agent. In some of any of the provided embodiments, the subject had progression of the disease within 24 months of diagnosis after completing treatment with an anti-CD20 antibody and an alkylating agent (POD24). In some of any of the provided embodiments, the subject had progression of the disease within 24 months of initiation of treatment with an anti-CD20 antibody and an alkylating agent (POD24).

In some embodiments, the subject has relapsed or is refractory to treatment after one prior line of therapy for treating FL Grade 1, 2 or 3A, and had progression of the disease within 24 months of initiation of the one prior line of therapy that is an anti-CD20 antibody and an alkylating agent (POD24). In some embodiments, the subject has relapsed or is refractory to treatment after one prior line of therapy for treating FL Grade 1, 2 or 3A, and had progression of the disease within 24 months of diagnosis after completing the one prior line of therapy that includes an anti-CD20 antibody and an alkylating agent (POD24).

In some embodiments, the subject has relapsed or is refractory to treatment after one prior line of therapy for treating FL Grade 1, 2 or 3A, and has at least one of the following: symptoms attributable to FL; threatened end-organ function, cytopenia secondary to lymphoma, or bulky disease; splenomegaly; and steady progression of disease over the preceding six months or more. In some embodiments, the subject has symptoms attributable to FL. In some embodiments, the subject has threatened end-organ function. In some embodiments, the subject has symptoms attributable to FL. In some embodiments, the subject has cytopenia secondary to lymphoma. In some embodiments, the subject has bulky disease. In some embodiments, bulky disease is a single mass>7 cm or 3 or more masses>3 cm. In some embodiments, the subject has splenomegaly. In some embodiments, the subject has steady progression of disease over the preceding six months or more.

In some embodiments, the at least one prior line of therapy is two prior lines of therapy. In some embodiments, the other of the two prior lines of therapy is selected from treatment with rituximab; obinutuzumab; bendamustine plus rituximab (BR); bendamustine plus obinutuzumab (BO); R-CHOP; rituximab, cyclophosphamide, vincristine, and prednisone (R-CVP); HSCT (optionally autologous or allogeneic HSCT); lenalidomide in combination with rituximab; or a PI3K inhibitor (PI3Ki). In some embodiments, the PI3Ki is idelalisib, copanlisib, or duvelisib.

In some embodiments, the subject is refractory to treatment or has relapsed within 12 months of completion of a prior line of therapy. In some embodiments, a prior line of therapy is a combination therapy or a monotherapy comprising a PI3Ki. In some embodiments, the other of the two prior lines of therapy is hematopoietic stem cell transplant (HSCT), and the subject has relapsed after HSCT.

In some of any of the provided embodiments, the subject is refractory to treatment with, or has relapsed during or up to 6 months after completing treatment with, an anti-CD20 therapy (anti-CD20 refractory). In some of any of the provided embodiments, the subject is refractory to treatment with an anti-CD20 therapy (anti-CD20 refractory). In some of any of the provided embodiments, the subject has relapsed during or up to 6 months after completing treatment with an anti-CD20 therapy (anti-CD20 refractory). In some of any of the provided embodiments, the subject is refractory to treatment with, or has relapsed during or up to 6 months after completing treatment with, an anti-CD20 antibody. In some of any of the provided embodiments, the subject is refractory to treatment with an anti-CD20 antibody. In some of any of the provided embodiments, the subject has relapsed during or up to 6 months after completing treatment with an anti-CD20 antibody.

In some of any of the provided embodiments, the subject is refractory to treatment with, or has relapsed during or up to 6 months after completing treatment with, an anti-CD20 therapy and an alkylating agent. In some of any of the provided embodiments, the subject is refractory to a prior line of therapy that includes an anti-CD20 therapy and an alkylating agent. In some of any of the provided embodiments, the subject has relapsed within six months of completing a prior line of therapy that includes an anti-CD20 therapy and an alkylating agent.

In some of any of the provided embodiments, the subject relapsed during an anti-CD20 antibody maintenance following 2 or more lines of therapy or within 6 months after maintenance completion. In some of any of the provided embodiments, the subject relapsed during an anti-CD20 antibody maintenance therapy following 2 or more lines of therapy. In some of any of the provided embodiments, the subject relapsed within 6 months after completion of the anti-CD20 antibody maintenance therapy. In some embodiments, the subject was treated with an anti-CD20 antibody maintenance therapy after the two prior lines of therapy, and the subject relapsed during the anti-CD20 maintenance therapy. In some embodiments, the subject was treated with an anti-CD20 antibody maintenance therapy, and the subject relapsed within six months of completing the anti-CD20 antibody maintenance therapy.

In some embodiments, the anti-CD20 antibody is a monoclonal antibody. In some of any such embodiments, the anti-CD20 monoclonal antibody is rituximab or obinutuzumab. In some of any such embodiments, the anti-CD20 monoclonal antibody is rituximab. In some of any such embodiments, the anti-CD20 antibody is obinutuzumab. In some of any such embodiments, the alkylating agent is bendamustine or chlorambucil. In some of any such embodiments, the alkylating agent is bendamustine. In some of any such embodiments, the alkylating agent is chlorambucil.

In some of any of the provided embodiments, the subject relapsed after hematopoietic stem cell transplant, optionally allogeneic or autologous HSCT. In some of any of the provided embodiments, the subject relapsed after hematopoietic stem cell transplant (HSCT). In some of any of the provided embodiments, the hematopoietic stem cell transplant is allogeneic HSCT. In some of any of the provided embodiments, the hematopoietic stem cell transplant is autologous HSCT.

In some of any of the provided embodiments, the at least one prior therapy is two prior lines of therapy. In some of any such embodiments, the other of the two prior lines of therapy is selected from treatment with rituximab; obinutuzumab; bendamustine plus rituximab (BR); bendamustine plus obinutuzumab (BO); R-CHOP; rituximab, cyclophosphamide, vincristine, and prednisone (R-CVP); HSCT (optionally autologous or allogeneic HSCT); lenalidomide in combination with rituximab; or a PI3K inhibitor, optionally wherein the PI3K inhibitor is idelalisib, copanlisib, or duvelisib. In some of any such embodiments, the other of the two prior lines of therapy is selected from treatment with rituximab; obinutuzumab; bendamustine plus rituximab (BR); bendamustine plus obinutuzumab (BO); R-CHOP; rituximab, cyclophosphamide, vincristine, and prednisone (R-CVP); HSCT (optionally autologous or allogeneic HSCT); lenalidomide in combination with rituximab; or a PI3K inhibitor. In some embodiments, the PI3K inhibitor is idelalisib, copanlisib, or duvelisib. In some embodiments, the PI3K inhibitor is idelalisib. In some embodiments, the PI3K inhibitor is copanlisib. In some embodiments, the PI3K inhibitor is duvelisib.

In some of any of the provided embodiments, the at least one prior therapy is three prior lines of therapy. In some of any such embodiments, the other two of the three prior lines of therapy are each independently selected from treatment with rituximab; obinutuzumab; bendamustine plus rituximab (BR); bendamustine plus obinutuzumab (BO); R-CHOP; rituximab, cyclophosphamide, vincristine, and prednisone (R-CVP); HSCT (optionally autologous or allogeneic HSCT); lenalidomide in combination with rituximab; or a PI3K inhibitor, optionally wherein the PI3K inhibitor is idelalisib, copanlisib, or duvelisib. In some of any such embodiments, the other two of the three prior lines of therapy are each independently selected from treatment with rituximab; obinutuzumab; bendamustine plus rituximab (BR); bendamustine plus obinutuzumab (BO); R-CHOP; rituximab, cyclophosphamide, vincristine, and prednisone (R-CVP); HSCT (optionally autologous or allogeneic HSCT); lenalidomide in combination with rituximab; or a PI3K inhibitor. In some embodiments, the PI3K inhibitor is idelalisib, copanlisib, or duvelisib. In some embodiments, the PI3K inhibitor is idelalisib. In some embodiments, the PI3K inhibitor is copanlisib. In some embodiments, the PI3K inhibitor is duvelisib.

In some embodiments, the subject is refractory to treatment with an anti-CD20 antibody. In some embodiments, the subject is refractory to a prior line of therapy that includes an anti-CD20 antibody and an alkylating agent. In some embodiments, the subject relapsed within six months of completing treatment with an anti-CD20 antibody. In some embodiments, the subject relapsed within six months of completing a prior line of therapy that includes an anti-CD20 antibody and an alkylating agent. In some embodiments, the subject was treated with an anti-CD20 antibody maintenance therapy after the two prior lines of therapy, and the subject relapsed during the anti-CD20 maintenance therapy or within six months of completing the anti-CD20 antibody maintenance therapy.

In some of any of the provided embodiments, the subject has relapsed or is refractory to treatment within 12 months of completion of a prior line of therapy, optionally after a treatment with a combination therapy or a monotherapy with a PI3Ki. In some of any of the provided embodiments, the subject has relapsed or is refractory to treatment within 12 months of completion of a combination therapy. In some of any of the provided embodiments, the subject has relapsed or is refractory to treatment within 12 months of completion of a monotherapy with a PI3Ki.

In some of any of the provided embodiments, the subject has relapsed or is refractory to treatment within 12 months of completion of a prior line of therapy. In some embodiments, the subject has relapsed or is refractory to treatment within 12 months of completion of a first line of therapy. In some embodiments, the subject has relapsed or is refractory to treatment within 12 months of completion of a second line of therapy. In some of any of the provided embodiments, the subject has relapsed within 12 months of completion of a prior line of therapy. In some of any of the provided embodiments, the subject has relapsed within 12 months of completion of a first line of therapy. In some of any of the provided embodiments, the subject has relapsed within 12 months of completion of a second line of therapy. In some of any of the provided embodiments, the subject is refractory to treatment within 12 months of completion of a prior line of therapy. In some of any of the provided embodiments, the subject is refractory to treatment within 12 months of completion of a first line of therapy. In some of any of the provided embodiments, the subject is refractory to treatment within 12 months of completion of a second line of therapy. In some embodiments, the prior line of therapy is treatment with a combination therapy or a monotherapy with a PI3Ki. In some embodiments, the prior line of therapy is monotherapy with a PI3Ki. In some embodiments, the prior line of therapy is treatment with a combination therapy.

In some embodiments, the anti-CD20 antibody is a monoclonal antibody. In some of any such embodiments, the anti-CD20 antibody is rituximab or obinutuzumab. In some of any such embodiments, the anti-CD20 antibody is rituximab. In some of any such embodiments, the anti-CD20 antibody is obinutuzumab. In some of any such embodiments, the alkylating agent is bendamustine or chlorambucil. In some of any such embodiments, the alkylating agent is bendamustine. In some of any such embodiments, the alkylating agent is chlorambucil.

In some of any of the provided embodiments, the subject has relapsed to the at least one prior line of therapy, and the relapse is after an initial response of complete response (CR) or partial response (PR) to the prior line of therapy. In some of any of the provided embodiments, the relapse is after an initial response of complete response (CR) to the prior line of therapy. In some of any of the provided embodiments, the relapse is after an initial response of partial response (PR) to the prior line of therapy. In some of any of the provided embodiments, the subject is refractory to treatment with the at least one or more prior therapy, and the refractory treatment is a best response of stable disease (SD) or progressive disease (PD) after the prior line of therapy. In some embodiments, the refractory treatment is a best response of stable disease (SD) after the prior line of therapy. In some embodiments, the refractory treatment is a best response of progressive disease (PD) after the prior line of therapy.

In some embodiments, the subject has relapsed to one prior line of therapy. In some embodiments, the subject has relapsed to two prior lines of therapy. In some embodiments, the subject has relapsed to three prior lines of therapy. In some embodiments, the subject has relapsed to more than three prior lines of therapy. In some embodiments, the subject is refractory to one prior line of therapy. In some embodiments, the subject is refractory to two prior lines of therapy. In some embodiments, the subject is refractory to three prior lines of therapy. In some embodiments, the subject is refractory to more than three prior lines of therapy.

In some of any of the provided embodiments, the subject has at least one PET-positive lesion and at least one measurable nodal lesion or extranodal lesion, optionally wherein the a measurable nodal lesion is greater than 1.5 cm in the long axis regardless of the short axis and a measurable extranodal lesion is greater than 1.0 cm in the long and short axis. In some of any of the provided embodiments, the subject has at least one PET-positive lesion and at least one measurable nodal lesion or extranodal lesion. In some of any of the provided embodiments, the a measurable nodal lesion is greater than 1.5 cm in the long axis regardless of the short axis and a measurable extranodal lesion is greater than 1.0 cm in the long and short axis.

Also provided herein is a method of treating marginal zone lymphoma (MZL), the method comprising administering to a subject having or suspected of having relapsed/refractory (r/r) marginal zone lymphoma (MZL) a dose of CD4+ and CD8+ T cells, wherein T cells of the dose comprises a chimeric antigen receptor (CAR) that specifically binds to CD19, wherein: the subject has relapsed or is refractory to treatment after at least two prior lines of therapy for treating MZL; the dose of T cells comprises between at or about 5×10⁷ CAR-expressing T cells and at or about 1.5×10⁸ CAR-expressing T cells, inclusive; the dose of T cells comprises a ratio of approximately 1:1 CD4+ T cells expressing the CAR to CD8+ T cells expressing the CAR; and the administration comprises administering a plurality of separate compositions, wherein the plurality of separate compositions comprises a first composition comprising the CD8+ CAR-expressing T cells and a second composition comprising the CD4+ CAR-expressing T cells.

Also provided hereis is use of a dose of CD4⁺ and CD8⁺ T cells to treat a subject having or suspected of having relapsed/refractory (r/r) marginal zone lymphoma (MZL), wherein: T cells of the dose include a chimeric antigen receptor (CAR) that specifically binds to CD19; the subject has relapsed or is refractory to treatment after at least two prior lines of therapy for treating MZL; the dose of T cells contains between at or about 5×10⁷ CAR-expressing T cells and at or about 1.5×10⁸ CAR-expressing T cells, inclusive; the dose of T cells has a ratio of approximately 1:1 CD4⁺ T cells expressing the CAR to CD8⁺ T cells expressing the CAR; and the dose is formulated for administration of a plurality of separate compositions, wherein the plurality of separate compositions includes a first composition containing the CD8⁺ CAR-expressing T cells and a second composition containing the CD4⁺ CAR-expressing T cells.

Also provided herein is use of a dose of CD4⁺ and CD8⁺ T cells in the manufacture of a medicament for treatment of a subject having or suspected of having relapsed/refractory (r/r) marginal zone lymphoma (MZL), wherein: T cells of the dose include a chimeric antigen receptor (CAR) that specifically binds to CD19; the subject has relapsed or is refractory to treatment after at least two prior lines of therapy for treating MZL; the dose of T cells contains between at or about 5×10⁷ CAR-expressing T cells and at or about 1.5×10⁸ CAR-expressing T cells, inclusive; the dose of T cells has a ratio of approximately 1:1 CD4⁺ T cells expressing the CAR to CD8⁺ T cells expressing the CAR; and the dose is formulated for administration of a plurality of separate compositions, wherein the plurality of separate compositions includes a first composition containing the CD8⁺ CAR-expressing T cells and a second composition containing the CD4⁺ CAR-expressing T cells.

In some of any of the provided embodiments, the MZL is a subtype selected from among extra-nodal MZL (ENMZL, mostly gastric), splenic MZL (SMZL), and nodal MZL (NMZL),In some embodiments, the MZL is extra-nodal MZL (ENMZL, mostly gastric). In some embodiments, the MZL is splenic MZL (SMZL). In some embodiments, the MZL is nodal MZL (NMZL).

In some of any of the provided embodiments, at least one of the at least two prior lines of therapy is a combination systemic therapy for treating the MZL or is a hematopoietic stem cell transplant (HSCT). In some of any of the provided embodiments, at least one of the at least two prior therapies is a combination systemic therapy for treating the MZL. In some of any of the provided embodiments, at least one of the at least two prior therapies is a hematopoietic stem cell transplant (HSCT).

In some of any of the provided embodiments, at least one of the at least two prior lines of therapy is a combination systemic therapy and the combination systemic therapy is selected from rituximab plus cyclophosphamide, doxorubicin, vincristine, and prednisone (R-CHOP), or a therapy with an anti-CD20 antibody and an alkylating agent. In some of any of the provided embodiments, at least one of the at least two prior lines of therapy is rituximab plus cyclophosphamide, doxorubicin, vincristine, and prednisone (R-CHOP). In some of any of the provided embodiments, at least one of the at least two prior lines of therapy is a combination systemic therapy that is an anti-CD20 antibody and an alkylating agent.

In some embodiments, the anti-CD20 antibody is a monoclonal antibody. In some of any such embodiments, the anti-CD20 antibody is rituximab or obinutuzumab. In some of any such embodiments, the anti-CD20 antibody is rituximab. In some of any such embodiments, the anti-CD20 antibody is obinutuzumab. In some of any such embodiments, the alkylating agent is bendamustine or chlorambucil .In some of any such embodiments, the alkylating agent is bendamustine. In some of any such embodiments, the alkylating agent is chlorambucil.

In some of any of the provided embodiments, at least one of the at least two prior therapies is rituximab and bendamustine or rituximab and chlorambucil. In some of any of the provided embodiments, at least one of the at least two prior lines of therapy is rituximab and bendamustine. In some of any of the provided embodiments, at least one of the at least two prior lines of therapy is rituximab and chlorambucil.

In some of any of the provided embodiments, the subject has splenic MZL (SMZL) and at least one of the at least two prior lines of therapy is a Splenectomy. In some of any of the provided embodiments, the subject has extra-nodal MZL (ENMZL) and an antibiotic is not one of the at least two prior lines of therapy.

In some of any of the provided embodiments, the subject has PET non-avid disease with at least one measurable nodal lesion greater than 2.0 cm in the long axis or at least one measurable extranodal lesion. In some of any of the provided embodiments, the subject has PET non-avid disease with at least one measurable nodal lesion greater than 2.0 cm in the long axis. In some of any of the provided embodiments, the subject has PET non-avid disease with at least one measurable extranodal lesion.

In some of any of the provided embodiments, the subject does not have FL grade 3B (FL3B). In some of any of the provided embodiments, the subject does not have evidence of composite DLBCL and FL, or of transformed FL. In some of any of the provided embodiments, the subject does not have evidence of composite DLBCL and FL. In some of any of the provided embodiments, the subject does not have evidence of transformed FL. In some of any of the provided embodiments, the subject does not have World Health Organization (WHO) subclassification of duodenal-type FL.

In some of any of the provided embodiments, the subject has relapsed to the at least one prior line of therapy, and the relapse is after an initial response of complete response (CR) or partial response (PR) to the prior line of therapy. In some of any of the provided embodiments, the relapse is after an initial response of complete response (CR) to the prior line of therapy. In some of any of the provided embodiments, the relapse is after an initial response of partial response (PR) to the prior line of therapy. In some of any of the provided embodiments, the subject is refractory to treatment with the at least one or more prior therapy, and the refractory treatment is a best response of stable disease (SD) or progressive disease (PD) after the prior line of therapy. In some embodiments, the refractory treatment is a best response of stable disease (SD) after the prior line of therapy. In some embodiments, the refractory treatment is a best response of progressive disease (PD) after the prior line of therapy.

In some embodiments, the subject has relapsed to one prior line of therapy. In some embodiments, the subject has relapsed to two prior lines of therapy. In some embodiments, the subject has relapsed to three prior lines of therapy. In some embodiments, the subject has relapsed to more than three prior lines of therapy. In some embodiments, the subject is refractory to one prior line of therapy. In some embodiments, the subject is refractory to two prior lines of therapy. In some embodiments, the subject is refractory to three prior lines of therapy. In some embodiments, the subject is refractory to more than three prior lines of therapy.

In some of any of the provided embodiments, the subject has Eastern Cooperative Oncology Group (ECOG) performance status of 0 or 1. In some of any of the provided embodiments, the subject has ECOG performance status of 0. In some of any of the provided embodiments, the subject has ECOG performance status of 1.

In some of any of the provided embodiments, prior to the administration of the dose of CD4+ and CD8+ T cells, the subject has been administered a lymphodepleting therapy. In some of any provided embodiments, the lymphodepleting therapy is completed within about 7 days prior to initiation of the administration of the dose of CD4+ and CD8+ T cells. In some of any provided embodiments, the lymphodepleting therapy is completed about 2 to 7 days prior to initiation of the administration of the dose of CD4+ and CD8+ T cells.

In some of any such embodiments, the lymphodepleting therapy comprises the administration of fludarabine and/or cyclophosphamide. In some of any such embodiments, the lymphodepleting therapy comprises the administration of fludarabine. In some of any such embodiments, the lymphodepleting therapy comprises the administration of cyclophosphamide. In some of any such embodiments, the lymphodepleting therapy comprises the administration of fludarabine and cyclophosphamide In some of any such embodiments, the lymphodepleting therapy comprises administration of cyclophosphamide at or about 200-400 mg/m², optionally at or about 300 mg/m², inclusive, and/or fludarabine at or about 20-40 mg/m², optionally 30 mg/m², daily for 2-4 days, optionally for 3 days. In some of any such embodiments, the lymphodepleting therapy comprises administration of cyclophosphamide at or about 300 mg/m², inclusive and/or fludarabine at or about 30 mg/m², daily for 3 days. In some of any such embodiments, the lymphodepleting therapy comprises administration of cyclophosphamide at or about 300 mg/m² and fludarabine at or about 30 mg/m² daily concurrently for 3 days.

In some of any of the provided embodiments, the CD19 is human CD19.

In some of any of the provided embodiments, the chimeric antigen receptor (CAR) comprises an scFv comprising the variable heavy chain region and the variable light chain region of the antibody FMC63, a spacer that is 15 amino acids of less and contains an immunoglobulin hinge region or a modified version thereof, a transmembrane domain, and an intracellular signaling domain comprising a signaling domain of a CD3-zeta (CD3ζ) chain and a costimulatory signaling region that is a signaling domain of 4-1BB.

In some of any such embodiments, the immunoglobulin hinge region or a modified version thereof comprises the formula X₁PPX₂P, wherein X₁ is glycine, cysteine or arginine and X₂ is cysteine or threonine (SEQ ID NO:58). In some embodiments, X₁ is glycine. In some embodiments, X₁ is cysteine. In some embodiments, X₁ is arginine. In some embodiments, X₂ is cysteine. In some embodiments, X₂ is threonine. In some of any such embodiments, the immunoglobulin hinge or a modified version thereof is an IgG1 hinge or a modified version thereof. In some of any such embodiments, the immunoglobulin hinge or a modified version thereof is an IgG1 hinge. In some of any such embodiments, the immunoglobulin hinge or a modified version thereof is modified version of an IgG1 hinge. In some of any such embodiments, the immunoglobulin hinge or a modified version thereof is an IgG4 hinge or a modified version thereof. In some of any such embodiments, the immunoglobulin hinge or a modified version thereof is an IgG4 hinge. In some of any such embodiments, the immunoglobulin hinge or a modified version thereof is a modified version of an IgG4 hinge.

In some of any such embodiments, the spacer comprises or consists of the sequence set forth in SEQ ID NO: 1, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto. In some of any such embodiments, the spacer is at or about 12 amino acids in length. In some of any such embodiments, the spacer comprises the sequence set forth in SEQ ID NO: 1, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity to the sequence set forth in SEQ ID NO: 1, SEQ ID NO: 30, SEQ ID NO: 31, SEQ ID NO: 32, SEQ ID NO: 33, or SEQ ID NO: 34. In some of any such embodiments, the spacer consists of the sequence set forth in SEQ ID NO: 1, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity to the sequence set forth in SEQ ID NO: 1, SEQ ID NO: 30, SEQ ID NO: 31, SEQ ID NO: 32, SEQ ID NO: 33, or SEQ ID NO: 34. In some of any such embodiments, the spacer comprises the sequence set forth in SEQ ID NO: 1. In some of any such embodiments, the spacer is at or about 12 amino acids in length. In some of any such embodiments, the spacer consists of the sequence set forth in SEQ ID NO: 1.

In some of any such embodiments, the transmembrane domain is a transmembrane domain of CD28. In some of any such embodiments, the transmembrane domain comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:8. In some of any such embodiments, the transmembrane domain comprises the sequence of amino acids set forth in SEQ ID NO: 8. In some of any such embodiments, the transmembrane domain consists of the sequence of amino acids set forth in SEQ ID NO: 8.

In some of any such embodiments, the costimulatory domain comprises the sequence set forth in SEQ ID NO: 12 or is a variant thereof having at least 85%, 86%, 87%, 88%, 89%, 90%, 91%, 92%, 93%, 94%, 95%, 96%, 97%, 98%, 99% or more sequence identity to the sequence set forth in SEQ ID NO:12. In some of any such embodiments, the costimulatory domain comprises the sequence set forth in SEQ ID NO: 12. In some of any such embodiments, the costimulatory domain consists of the sequence set forth in SEQ ID NO: 12. In some of any such embodiments, the signaling domain of a CD3-zeta (CD3ζ) chain comprises the sequence set forth in SEQ ID NO: 13, 14, or 15, or is a variant thereof having at least 85%, 86%, 87%, 88%, 89%, 90%, 91%, 92%, 93%, 94%, 95%, 96%, 97%, 98%, 99% or more sequence identity to the sequence set forth in SEQ ID NO:13, 14 or 15. In some of any such embodiments, the signaling domain of a CD3-zeta (CD3ζ) chain comprises the sequence set forth in SEQ ID NO: 13. In some of any such embodiments, the signaling domain of a CD3-zeta (CD3ζ) chain comprises the sequence set forth in SEQ ID NO: 14. In some of any such embodiments, the signaling domain of a CD3-zeta (CD3ζ) chain comprises the sequence set forth in SEQ ID NO: 15. In some of any such embodiments, the signaling domain of a CD3-zeta (CD3ζ) chain consists of the sequence set forth in SEQ ID NO: 13. In some of any such embodiments, the signaling domain of a CD3-zeta (CD3ζ) chain consists of the sequence set forth in SEQ ID NO: 14. In some of any such embodiments, the signaling domain of a CD3-zeta (CD3ζ) chain consists of the sequence set forth in SEQ ID NO: 15.

In some of any such embodiments, the scFv comprises a CDRL1 sequence of SEQ ID NO: 35, a CDRL2 sequence of SEQ ID NO: 55, and/or a CDRL3 sequence of SEQ ID NO: 56 and a CDRH1 sequence of SEQ ID NO: 38, a CDRH2 sequence of SEQ ID NO: 39, and/or a CDRH3 sequence of SEQ ID NO: 54. In some of any such embodiments, the scFv comprises a CDRL1 sequence of SEQ ID NO: 35, a CDRL2 sequence of SEQ ID NO: 55, and a CDRL3 sequence of SEQ ID NO: 56 and a CDRH1 sequence of SEQ ID NO: 38, a CDRH2 sequence of SEQ ID NO: 39, and a CDRH3 sequence of SEQ ID NO: 54. In some of any such embodiments, the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40). In some of any such embodiments, the scFv comprises a CDRL1 sequence of RASQDISKYLN (SEQ ID NO: 35), a CDRL2 sequence of SRLHSGV (SEQ ID NO: 36), and a CDRL3 sequence of GNTLPYTFG (SEQ ID NO: 37) and a CDRH1 sequence of DYGVS (SEQ ID NO: 38), a CDRH2 sequence of VIWGSETTYYNSALKS (SEQ ID NO: 39), and a CDRH3 sequence of YAMDYWG (SEQ ID NO: 40). In some of any such embodiments, the scFv comprises, in order, a V_{H}, comprising the sequence set forth in SEQ ID NO: 41, and a V_{L} comprising the sequence set forth in SEQ ID NO: 42. In some of any such embodiments, the scFv comprises the sequence set forth in SEQ ID NO:43. In some embodiments, the scFv comprises, in order from N-terminus to C-terminus, a V_{L} comprising the sequence set forth in SEQ ID NO: 42, and a V_{H}, comprising the sequence set forth in SEQ ID NO: 41.

In some of any of the provided embodiments, the CAR contains in order from N-terminus to C-terminus an extracellular antigen-binding domain that is the scFv set forth in SEQ ID NO: 43, the spacer set forth in SEQ ID NO:1, the transmembrane domain set forth in SEQ ID NO:8, the 4-1BB costimulatory signaling domain set forth in SEQ ID NO:12, and the signaling domain of a CD3-zeta (CD3ζ) chain set forth in SEQ ID NO:13.

In some embodiments, the dose of CD4+ and CD8+ T cells is between about 5×10⁷ CAR+ T cells and about 1.1×10⁸ CAR+ T cells, inclusive of each. In some of any of the provided embodiments, the dose of CD4+ and CD8+ T cells is 5×10⁷CAR+ T cells. In some of any of the provided embodiments, the dose of CD4+ and CD8+ T cells is 6×10⁷CAR+ T cells. In some of any of the provided embodiments, the dose of CD4+ and CD8+ T cells is 7×10⁷CAR+ T cells. In some of any of the provided embodiments, the dose of CD4+ and CD8+ T cells is 8×10⁷CAR+ T cells. In some of any of the provided embodiments, the dose of CD4+ and CD8+ T cells is 9×10⁷ CAR+ T cells. In some of any of the provided embodiments, the dose of CD4+ and CD8+ T cells is 1×10⁸ CAR+ T cells. In some of any of the provided embodiments, the dose of CD4+ and CD8+ T cells is 1.1×10⁸ CAR+ T cells. In some of any of the provided embodiments, the dose of CD4+ and CD8+ T cells is 1.2×10⁸ CAR+ T cells. In some of any of the provided embodiments, the dose of CD4+ and CD8+ T cells is 1.3×10⁸ CAR+ T cells. In some of any of the provided embodiments, the dose of CD4+ and CD8+ T cells is 1.4×10⁸ CAR+ T cells. In some of any of the provided embodiments, the dose of CD4+ and CD8+ T cells is 1.5×10⁸ CAR+ T cells.

In some of any of the provided embodiments, the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or the initiation of administration of the first composition and the initiation of administration of the second composition are carried out between about 1 minute and about 1 hour apart or between about 5 minutes and about 30 minutes apart. In some of any of the provided embodiments, the first composition and second composition are administered 0 to 12 hours apart. In some of any of the provided embodiments, the first composition and second composition are administered 0 to 6 hours apart. In some of any of the provided embodiments, the first composition and second composition are administered 0 to 2 hours apart. In some of any of the provided embodiments, the administration of the first composition and the administration of the second composition are carried out on the same day. In some of any of the provided embodiments, the administration of the first composition and the administration of the second composition are carried out between about 0 and about 12 hours apart. In some of any of the provided embodiments, the administration of the first composition and the administration of the second composition are carried out between about 0 and about 6 hours apart. In some of any of the provided embodiments, the administration of the first composition and the administration of the second composition are carried out between about 0 and 2 hours apart. In some embodiments, the initiation of administration of the first composition and the initiation of administration of the second composition are carried out between about 1 minute and about 1 hour apart. In some embodiments, the initiation of administration of the first composition and the initiation of administration of the second composition are carried out between about 5 minutes and about 30 minutes apart. In some of any of the provided embodiments, the first composition and second composition are administered no more than 2 hours, no more than 1 hour, no more than 30 minutes, no more than 15 minutes, no more than 10 minutes or no more than 5 minutes apart. In some of any of the provided embodiments, the first composition and second composition are administered no more than 2 hours apart. In some of any of the provided embodiments, the first composition and second composition are administered less than 2 hours apart. In some of any of the provided embodiments, the first composition and second composition are administered no more than 1 hour apart. In some of any of the provided embodiments, the first composition and second composition are administered no more than 30 minutes apart. In some of any of the provided embodiments, the first composition and second composition are administered no more than 15 minutes apart. In some of any of the provided embodiments, the first composition and second composition are administered no more than 10 minutes apart. In some of any of the provided embodiments, the first composition and second composition are administered no more than 5 minutes apart. In some of any of the provided embodiments, the first composition is administered prior to the second composition. In some embodiments, the first composition comprises the CD8+ CAR-expressing T cells and the second composition comprises the CD4+ T CAR-expressing cells.

In some of any of the provided embodiments, the dose of CD4+ T cells and CD8+ T cells is administered intravenously. In some of any of the provided embodiments, the T cells are primary T cells obtained from the sample from the subject. In some of any of the provided embodiments, the T cells are autologous to the subject. In some embodiments, the sample from which the cells are derived or isolated is a blood or a blood-derived sample. In some embodiments, the sample from which the cells are derived or isolated is or is derived from an apheresis or leukapheresis product. In some embodiments, the sample is obtained from the subject prior to administration of a lymphodepleting therapy to the subject. In some embodiments, a leukapheresis sample is obtained from a subject approximately four or five weeks prior to administration of the dose of CD4+ T cells and CD8+ T cells to the subject. In some of any such embodiments, the subject is human

In some of any of the provided embodiments, the complete response rate (CRR) among a plurality of subjects treated according to the method is greater than at or about 50%, greater than at or about 60%, greater than at or about 70%, or greater than at or about 80%. In some of any of the provided embodiments, the complete response rate (CRR) among a plurality of subjects treated according to the method is greater than at or about 50%. In some of any of the provided embodiments, the complete response rate (CRR) among a plurality of subjects treated according to the method is greater than at or about 60%. In some of any of the provided embodiments, the complete response rate (CRR) among a plurality of subjects treated according to the method is greater than at or about 70%. In some of any of the provided embodiments, the complete response rate (CRR) among a plurality of subjects treated according to the method is greater than at or about 80%. In some of any such embodiments, CRR is the percentage of subjects with a best overall response (BOR) up to 24 months of complete response (CR).

In some of any of the provided embodiments, the subject has FL Grade 1, 2 or 3A and CRR is assessed by PET-CT. In some of any of the provided embodiments, the subject has FL Grade 1 and CRR is assessed by PET-CT. In some of any of the provided embodiments, the subject has FL Grade 2 and CRR is assessed by PET-CT. In some of any of the provided embodiments, the subject has FL Grade 3A and CRR is assessed by PET-CT. In some of any of the provided embodiments, the subject has MZL and CRR is assessed by CT. In some of any of the provided embodiments, the subject has MZL and CRR is assessed by MRI. In some of any of the provided embodiments, the subject has MZL and CRR is assessed by CT and MRI scans.

### BRIEF DESCRIPTION OF THE DRAWINGS

FIG. 1A shows differential gene expression profiles in pre-treatment tumor biopsies in subjects showing complete response (CR) or progressive disease (PD) at 3 months post-treatment, among patients in the initial cohort.

FIG. 1B shows various enriched gene sets associated with PD at 3 months post-treatment among patients in the initial cohort, including genes expressed more highly in diffuse large B-cell lymphoma (DLBCL) cell line samples compared to follicular lymphoma cell line samples (FL; FL_DLBCL_DN).

FIG. 2A shows differential gene expression profiles in pre-treatment tumor biopsies in subjects showing complete response (CR) or progressive disease (PD) at 3 months post-treatment, among patients in the larger cohort.

FIG. 2B shows various enriched gene sets associated with PD at 3 months post-treatment among patients in the larger cohort, including genes expressed more highly in diffuse large B-cell lymphoma (DLBCL) compared to follicular lymphoma (FL; DLBCL_LIKE_vs_FL and SHIPP_DLBCL_VS_FOLLICULAR_LYMPHOMA_UP).

FIG. 2C shows various enriched gene sets associated with T cell exclusion among matched pre-treatment and day 11 post-treatment samples, including genes expressed more highly in diffuse large B-cell lymphoma (DLBCL) compared to follicular lymphoma (FL; DLBCL_LIKE_vs_FL and SHIPP_DLBCL_VS_FOLLICULAR_LYMPHOMA_UP).

FIG. 3 shows differential gene expression between FL tumor biopsies and DLBCL tumor biopsies.

FIGS. 4A and 4B show differential gene expression of exemplary genes EZH2 (FIG. 4A) and CD3ε (FIG. 4B) between FL and DLBCL tumors.

FIGS. 5A and 5B (initial cohort) and FIG. 5C (larger cohort) show the single-sample Gene Set Enrichment Analysis (ssGSEA) scores between genes found to be elevated in DLBCL (designated “DLBCL-like gene set”; FIG. 5A) versus in FL (designated “FL-like gene set”; FIGS. 5B and 5C) and subjects who went onto exhibit a CR or subjects who went onto exhibit PD, and illustrates that subjects having tumor gene expression profiles more similar to those seen in FL, as compared to those seen in DLBCL, were more likely to show CR at 3 months post-treatment.

FIG. 5D shows progression free survival (PFS) curves among 74 DLBCL subjects, compared between the 15 subjects with the highest FL-like gene expression and the other 59 subjects.

### DETAILED DESCRIPTION

Provided are methods and uses of engineered cells (e.g., T cells) and/or compositions thereof, for the treatment of subjects having a disease or condition, which generally is or includes a cancer or a tumor, such as a leukemia or a lymphoma, most particularly a B cell malignancy or a non-Hodgkin lymphoma (NHL). In particular embodiments of any of the provided methods, the T cells are engineered with a chimeric antigen receptor (CAR) that is directed against CD19. In some aspects, the disease or condition is a B cell lymphoma. In some aspects, the disease or condition is a follicular lymphoma (FL), grade 1-3A. In some aspects, the disease or condition is a marginal zone lymphoma (MZL). In some aspects, the methods and uses provide for or achieve improved response and/or more durable responses or efficacy and/or a reduced risk of toxicity or other side effects, e.g., in particular groups of subjects treated, as compared to certain alternative methods. In some embodiments, the methods are advantageous by virtue of the administration of specified numbers or relative numbers of the engineered cells, the administration of defined ratios of particular types of the cells, treatment of particular patient populations, such as those having a particular risk profile, staging, and/or prior treatment history, and/or combinations thereof.

In some embodiments, the methods and uses include administering to the subject cells expressing genetically engineered (recombinant) cell surface receptors in adoptive cell therapy, which generally are chimeric receptors such as chimeric antigen receptors (CARs), recognizing an antigen expressed by, associated with and/or specific to the lymphoma and/or cell type from which it is derived. The cells are generally administered in a composition formulated for administration; the methods generally involve administering one or more doses of the cells to the subject, which dose(s) may include a particular number or relative number of cells or of the engineered cells, and/or a defined ratio or compositions of two or more sub-types within the composition, such as CD4 vs. CD8 T cells.

In some embodiments, the cells, populations, and compositions are administered to a subject having the particular disease or condition to be treated, e.g., via adoptive cell therapy, such as adoptive T cell therapy. In some embodiments, the methods involve treating a subject having a lymphoma or a B cell malignancy, such as an indolent non-Hodgkin lymphoma (NHL) with a dose of antigen receptor-expressing cells (e.g. CAR-expressing cells).

In some embodiments, the provided methods involve treating a specific group or subset of subjects, e.g., subjects identified as having high-risk disease. In some aspects, the methods treat subjects having a poor prognosis indolent NHL, such as NHL that has relapsed or is refractory (R/R) to standard therapy and/or has a poor prognosis. In some aspects, the methods treat subjects having a NHL such as FL G1-3A or MZL, that has relapsed or is refractory (R/R) to standard therapy. In some aspects, the provided methods, compositions, uses and articles of manufacture achieve improved and superior responses to available therapies. In some embodiments, the improved or superior responses are compared to current standard of care (SOC).

CD19 is a 95 kDa glycoprotein present on B cells from early development until differentiation into plasma cells (Stamenkovic et al., J Exp Med. 1988; 168(3):1205-10). It is a member of the immunoglobulin superfamily and functions as a positive regulator of the B-cell receptor by lowering the signaling threshold for B-cell activation (Brentjens et al., Blood. 2011; 118(18):4817-28; LeBien et al, Blood. 2008; 112(5):1570-80). CD19 is an attractive therapeutic target because it is expressed by most B-cell malignancies, including B-cell NHL (Davila et al., Oncoimmunology. 2012; (9):1577-83) Importantly, the CD19 is not expressed on hematopoietic stem cells or on any normal tissue apart from those of the B-cell lineage. Additionally, CD19 is not shed in the circulation, which limits off-target adverse effects (Shank et al., Pharmacotherapy. 2017; 37(3):334-45).

In particular embodiments, the methods provided herein are based on administration of a CD19-directed CAR T cell therapy in which the CAR contains a CD19-directed scFv antigen binding domain (e.g. from FMC63). The CAR further contains an intracellular signaling domain containing a signaling domain from CD3zeta, and also incorporates a 4-1BB costimulatory domain, which has been associated with lower incidence of cytokine release syndrome (CRS) and neurotoxicity (NT; e.g. neurological events (NE)) compared with CD28-containing constructs (Lu et al. J Clin Oncol. 2018; 36:3041). In some embodiments, the methods provided herein include CD8+ and CD4+ T-cell subsets that are transduced and expanded separately in vitro, and administered at equal (about 1:1) target doses. In some embodiments, there is low variability in the administered total CD4+ and CD8+ CAR+ T-cell doses, two parameters associated with increased toxicity in previous studies (Neelapu et al., N Engl Med. 2017. 377; 2531-44; Turtle et al., Sci Transl Med. 2016; 8:355ra116; Hay et al., Blood. 2017; 130:2295-306).

In some embodiments, the methods include administration of cells to a subject selected or identified as having a certain prognosis or risk of a B cell malignancy, such as a NHL, such as FL G1-3A, or MZL. Lymphomas, such as NHL, can be a variable disease. Some subjects with NHL may survive without treatment while others may require immediate intervention. In some embodiments, the methods, uses and articles of manufacture involve, or are used for treatment of subjects involving, selecting or identifying a particular group or subset of subjects, e.g., based on specific types of disease, diagnostic criteria, prior treatments and/or response to prior treatments. In some embodiments, the methods involve treating a subject having relapsed following remission after treatment with, or become refractory to, one or more prior therapies; or a subject that has relapsed or is refractory (R/R) to one or more prior therapies, e.g., one or more lines of standard therapy. In some embodiments, the methods involve treating a subject having relapsed following remission after treatment with, or become refractory to, two or more prior therapies. In some embodiments, the methods involve treating a subject that has relapsed or is refractory (R/R) to two or more prior therapies, e.g., two or more lines of standard therapy. In some embodiments, the methods involve treating a subject having relapsed following remission after treatment with, or become refractory to, three or more prior therapies; or a subject that has relapsed or is refractory (R/R) to three or more prior therapies, e.g., three or more lines of standard therapy.

In some embodiments, the subject has been previously treated with a therapy or a therapeutic agent targeting the disease or condition, e.g., a NHL such as FL G1-3A or MZL, prior to administration of the cells expressing the recombinant receptor. In some embodiments, the subject has been previously treated with a hematopoietic stem cell transplantation (HSCT), e.g., allogeneic HSCT or autologous HSCT. In some embodiments, the subject has had poor prognosis after treatment with standard therapy and/or has failed one or more lines of previous therapy. In some embodiments, the subject has been treated or has previously received at least or at least about or about 1, 2, 3, 4, or more other therapies for treating the disease or disorder, such as FL G1-3A or MZL, other than a lymphodepleting therapy and/or the dose of cells expressing the antigen receptor. In some embodiments, the subject has been treated or has previously received a therapy that includes a CD20 targeted agent (e.g. anti-CD20 antibody) and an alkylating agent.

Certain CD19-directed CAR-T cell therapies are available for treatment of B cell lymphoma, including axicabtagene ciloleucel (axi-cel) and tisagenlecleucel. In one exemplary study, axi—cel-treated subjects achieved CR rates of 54%, with 40% in durable remission (median follow-up, 15.4 months) (Neelapu et al., N Engl Med. 2017. 377; 2531-44). Most subjects developed CRS (93%) and NEs (64%), with median time to onset of 2 and 5 days, respectively, and grade ≥3 CRS (Lee grading criteria (Lee et al., Blood. 2014; 124:188-95) and NEs occurred in 13% and 28%, respectively, and 43% received tocilizumab (27% received corticosteroids). In another exemplary study, approximately one-third of patients who received tisagenlecleucel maintained durable remission at 1 year (Schuster et al., N Engl J Med. 2019. 380:45-56). Most subjects (58%) developed CRS, while 21% had NEs. Grade ≥3 CRS (Penn grading criteria, Porter et al., J Hematol Oncol. 2018; 11:35) and NEs were reported in 22% and 12% of patients, respectively (Schuster et al., N Engl J Med. 2019. 380:45-56). Although favorable response outcomes were observed for treating these indications, the toxicity rates remain high. Further, there currently are no approved CAR T-cell therapies for treating patients with certain lymphoma subtypes including patients with FL G1-3A or MZL, and in particular in such patients with certain high-risk features, such as having a double refractory status or disease progression within 24 months of initial diagnosis and/or initiation of treatment (POD24).

FL is the most common subtype of indolent non-Hodgkin lymphomas (NHL), accounting for about 10% to 20% of all lymphoma cases in Western countries (Union for International Cancer Control [UICC], 2014; Miranda-Filho et al., Cancer Causes Control. 2010: 30(5):489-99). Second-line (2L) treatments most commonly include the same treatments used in frontline, mainly chemoimmunotherapy regimens. After 2L therapy in FL, options for r/r FL patients are limited. Both the National Comprehensive Cancer Network (NCCN, 2019) and the European Society for Medical Oncology (ESMO) (Dreyling, 2016) guidelines suggest that if a long remission was achieved in the frontline setting, often the same regimen is repeated in 2L treatment, most frequently for patients that receive bendamustine plus rituximab (BR) in frontline and less frequently for patients that receive rituximab, cyclophosphamide, vincristine, doxorubicin, and prednisolone (R-CHOP), due to concern for cardiotoxicity. Second-line treatments for highly selected patients may also include autologous or allogeneic hematopoietic stem cell transplant (HSCT) (NCCN, 2019; Dreyling et al., 2016 Ann Oncol. 2016; 27(83-v90). Currently, there is no standard of care in fourth-line FL and beyond (4L+). No agents are currently approved in this setting. Moreover, by fourth-line, patients have generally exhausted available treatment options. Progression of the disease results in shorter intervals between each treatment and failure, suggesting that patients who have received three prior systemic therapies at time of disease relapse or progression would comprise a patient population of high unmet need. The lack of available treatment options in the refractory setting presents a significant unmet need, particularly in the third-line and beyond (3L+). While the prognosis for FL has improved substantially over the past two decades, FL remains incurable (Kahl et al., Blood. 2016 Apr. 28; 127(17):2055-63).

Marginal zone lymphoma (MZL) is the third most common NHL histology accounting for 8% to 12% of all B-cell NHL cases (Swerdlow et al., 2016; Blood, 30:84-91; Al-Hamadani et al., Am J Hematol., 2015; 90(9):790-5). Three subtypes are described: extra-nodal MZL (ENMZL, mostly gastric), splenic MZL (SMZL), and nodal MZL (NMZL). Patients with MZL are often underrepresented or excluded from studies of other indolent NHL, given its heterogeneity. Moreover, treatment-related toxicities may limit options for older patients (median age 67) and the clinical course of MZL is also heterogeneous, with nodal disseminated MZL associated with worse prognosis compared to other subtypes (Denlinger et al., Cancer Manag Res. 2018; 10:615-24). Ibrutinib is a common therapy for MZL, such that by third-line therapy (3L) many patients with MZL have likely been treated with ibrutinib, and thus patients failing two prior lines of therapy are considered a high unmet need population. Other treatment options include lenalidomide and rituximab combination in 2L+ MZL.

Available evidence indicates a high unmet need for additional therapeutic options that provide a favorable benefit/risk profile and durable response, which is not met with available therapies for subjects with second-line (2L), third-line (3L) or fourth-line and beyond (4L+) indolent FL Grade 1-3A. Furthermore, limited treatment options are available for the rarer subtypes of NHL, including MZL. These findings reveal important treatment gaps for R/R NHL, especially among subjects with high-risk features of FL G1-3A or MZL, and a need for improvement of the existing treatments. Provided herein are embodiments that can meet such needs.

In particular embodiments, the provided methods can be used to treat particular NHL subtypes or high-risk groups, such as patients who are double refractory to treatment or have POD24, in which available treatment options remain limited. In some cases, available therapies including other CAR T-cell therapies, may be associated with high risk of toxicity. In some aspects, cytokine-associated toxicity such as CRS and neurotoxicity have been observed with CAR T-cell therapies (Bonifant et al., Mol Ther Oncolytics. 2016; 3:16011; Turtle et al., J Clin Invest. 2016; 126(6):2123-38). For example, existing CAR T-cell therapies are associated with severe CAR T-cell-related toxicities, including cytokine release syndrome (CRS) and neurological events (NEs), that may limit administration to specialized treatment center (Yescarta Risk Evaluation and Mitigation Strategy (REMS) Gilead Pharma Sept. 10, 2019; Kymriah Risk Evaluation and Mitigation Strategy (REMS) Novartis Sep. 10, 2019) and impact use in difficult-to-treat patients. CAR T-cell therapies with a favorable benefit/risk profile may allow for broader inclusion of subject subgroups including high-risk patient populations.

The observations herein support treating subjects with high-risk disease with a CD19-directed CAR T-cell therapy in accord with the provided methods. For example, subjects with FL Grade 1-3A or MZL and patients with certain high-risk features, such as having double refractory status or disease progression within 24 months of initial diagnosis and/or initiation of treatment (POD24), can be treated in accord with the provided methods. In some embodiments, the provided methods can be used to treat subjects that have been heavily pretreated (e.g. with one, two, three, four, or more prior therapies for treating the disease).

Thus, in some embodiments, the provided methods, articles of manufacture and/or compositions, can offer advantages over other available methods or solutions or approaches for treatment such as for adoptive cell therapy. In particular, among the provided embodiments are those that offer a treatment for subjects that have R/R FL G1-3A and R/R MZL. In particular embodiments, among subjects with R/R FL G1-3A subjects that have a double refractory status or disease progression within 24 months of initial diagnosis and/or initiation of treatment (POD24).

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. METHODS AND USES OF CD19-TARGETED CELL THERAPY IN INDOLENT FOLLICULAR LYMPHOMA (FL) GRADE 1-3A AND MARGINAL ZONE LYMPHOMA (MZL)

Provided herein are methods of treatment that involve administering engineered cells or compositions containing engineered cells, such as engineered T cells. Also provided are methods and uses of engineered cells (e.g., T cells) and/or compositions thereof, including methods for the treatment of subjects having R/R FL G1-3A or R/R MZL, that involves administration of the engineered cells and/or compositions thereof. In some aspects, also provided are uses of engineered cells or compositions containing engineered cells, such as engineered T cells for treatment of R/R FL G1-3A or R/R MZL. In some aspects, the uses of the engineered cells or compositions containing engineered cells, such as engineered T cells are in accord with any of the methods described herein.

In some embodiments, the methods and uses include administering to the subject cells expressing genetically engineered (recombinant) cell surface receptors in adoptive cell therapy, which generally are chimeric receptors such as chimeric antigen receptors (CARs), recognizing an antigen expressed by, associated with and/or specific to the lymphoma and/or cell type from which it is derived. The cells are generally administered in a composition formulated for administration. In particular embodiments, the methods involve administering one or more doses of the cells to the subject that include a particular number or relative number of cells or of the engineered cells, such as a defined ratio or compositions of two or more sub-types within the composition, such as CD4 vs. CD8 T cells. In some embodiments, the engineered cells or compositions comprising the same are administered in an effective amount to effect treatment of the disease or disorder. Uses include uses of the engineered cells or compositions in such methods and treatments, and in the preparation of a medicament in order to carry out such therapeutic methods. In some embodiments, the methods are carried out by administering the engineered cells, or compositions comprising the same, to the subject having or suspected of having the disease or condition. In some embodiments, the methods thereby treat the disease or condition or disorder in the subject.

General methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cells, populations, and compositions are administered to a subject having an indolent NHL to be treated, e.g., via adoptive cell therapy, such as adoptive T cell therapy. In some aspects, NHL can be staged based on the Lugano classification (see, e.g., Cheson et al., (2014) JCO 32(27):3059-3067; Cheson, B. D. (2015) Chin Clin Oncol 4(1):5). In some cases, the stages are described by Roman numerals I through IV (1-4), and limited stage (I or II) lymphomas that affect an organ outside the lymph system (an extranodal organ) are indicated by an E. Stage I represents involvement in one node or a group of adjacent nodes, or a single extranodal lesions without nodal involvement (IE). Stage 2 represents involvement in two or more nodal groups on the same side of the diaphragm or stage I or II by nodal extent with limited contiguous extranodal involvement (IIE). Stage III represents involvement in nodes on both sides of the diaphragm or nodes above the diaphragm with spleen involvement. Stage IV represents involvement in additional non-contiguous extra-lymphatic involvement. In addition, “bulky disease” can be used to describe large tumors in the chest, in particular for stage II. The extent of disease is determined by positron emission tomography (PET)-computed tomography (CT) for avid lymphomas, and CT for non-avid histologies. In some of any embodiments, at or prior to the administration of the dose of cells, the subject to be treated according to the provided embodiments has a positron emission tomography (PET)-positive disease.

In some embodiments, the methods involve treating a subject having a R/R FL G1-3A or R/R MZL with a dose of antigen receptor-expressing cells (e.g. CAR-expressing cells). In particular embodiments, the method includes administering to the subject a dose of CD4+ and CD8+ T cells, wherein T cells of each dose comprises a chimeric antigen receptor (CAR) that specifically binds to CD19.

In some embodiments, the subject is 18 years of age or older. In some embodiments, if the subject has previously received a CD19-targeted therapy, the subject must be confirmed to have CD19-positive lymphoma since completing the prior CD19-targeting therapy. In some embodiments, the subject has histologically confirmed disease within 6 months of screening. In some embodiments, the subject has adequate organ function and vascular access.

In some embodiments, the subject has an Eastern Cooperative Oncology Group (ECOG) performance status of 0 or 1. In some embodiments, the Eastern Cooperative Oncology Group (ECOG) performance status indicator can be used to assess or select subjects for treatment, e.g., subjects who have had poor performance from prior therapies (see, e.g., Oken et al., (1982) Am J Clin Oncol. 5:649-655). The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of self-care; see e.g., Sørensen et al., (1993) Br J Cancer 67(4) 773-775. The criteria reflective of the ECOG performance status are described in Table 1 below:

TABLE 1
ECOG Performance Status Criteria

| Grade | ECOG performance status |
|---|---|
| 0 | Fully active, able to carry on all pre-disease performance without restriction |
| 1 | Restricted in physically strenuous activity but ambulatory and able to carry out |
| | work of a light or sedentary nature, e.g., light house work, office work |
| 2 | Ambulatory and capable of all self-care but unable to carry out any work |
| | activities; up and about more than 50% of waking hours |
| 3 | Capable of only limited self-care; confined to bed or chair more than 50% of |
| | waking hours |
| 4 | Completely disabled; cannot carry on any self-care; totally confined to bed or |
| | chair |
| 5 | Dead |

The provided methods are for treatment of subjects that have relapsed or are refractory to (R/R) a prior therapy. In some aspects, the subject has relapsed after an initial response of complete response (CR) or partial response (PR) to the prior therapy. In some embodiments, the subject is refractory to treatment with the at least one or more prior therapy, and the refractory treatment is a best response of stable disease (SD) or progressive disease (PD) after the prior therapy.

In some embodiments, the subject is at least 18 years of age. In particular embodiments, the provided methods can result in favorable outcomes and low toxicity rates in a group of subjects that are older, including in subjects greater than 60 years of age or older.

In some embodiments, the provided methods implement flat dosing, e.g. total number of CAR+ cells, total number of CAR+CD8+ T cells and/or CAR+CD4+ T cells, such as to administer a precise or fixed dose of such cell type(s) to each of a group of subjects treated, including subjects of variable weight. Thus, the provided methods include methods in which the dose of cells is a flat dose of cells or fixed dose of cells such that the dose of cells is not tied to or based on the body surface area or weight of a subject. In some embodiments, such methods minimize or reduce the chance of administering too many cells to the subject, which may increase the risk of a toxic outcome associated with administration of the CAR-T cells.

In some embodiments, the dose of T cells comprises between about 5×10⁷ recombinant receptor (e.g. CAR)-expressing T cells and about 1.1×10⁸ recombinant receptor (e.g. CAR)-expressing T cells. In some embodiments, the dose of T cells comprises at or about 5×10⁷ recombinant receptor (e.g. CAR)-expressing T cells. In some embodiments, the dose of T cells comprises at or about 6×10⁷ recombinant receptor (e.g. CAR)-expressing T cells. In some embodiments, the dose of T cells comprises at or about 7×10⁷ recombinant receptor (e.g. CAR)-expressing T cells. In some embodiments, the dose of T cells comprises at or about 0.75×10⁸ recombinant receptor (e.g. CAR)-expressing CD8⁺ T cells. In some embodiments, the dose of T cells comprises at or about 8×10⁷ recombinant receptor (e.g. CAR)-expressing T cells. In some embodiments, the dose of T cells comprises at or about 9×10⁷ recombinant receptor (e.g. CAR)-expressing T cells. In some embodiments, the dose of T cells comprises at or about 1×10⁸ recombinant receptor (e.g. CAR)-expressing T cells. In some embodiments, the dose of T cells comprises at or about 1.1×10⁸ recombinant receptor (e.g. CAR)-expressing T cells. In some embodiments, the dose of T cells comprises at or about 1.5×10⁸ recombinant receptor (e.g. CAR)-expressing T cells. In some embodiments, the T cells of the dose include CD4⁺ and CD8⁺ T cells. In some embodiments, the number of cells is the number of such cells that are viable cells.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8⁺- and CD4⁺-enriched populations, respectively, e.g., CD4⁺ and/or CD8⁺ T cells each individually including cells genetically engineered to express the recombinant receptor. In some embodiments, the administration of the dose comprises administration of a first composition comprising a dose of CD8⁺ T cells or a dose of CD4⁺ T cells and administration of a second composition comprising the other of the dose of CD4⁺ T cells and the CD8⁺ T cells.

In some embodiments, the administration of the composition or dose, e.g., administration of the plurality of cell compositions, involves administration of the cell compositions separately. In some aspects, the separate administrations are carried out simultaneously, or sequentially, in any order. In particular embodiments, the separate administrations are carried out sequentially by administering, in any order, a first composition comprising a dose of CD8⁺ T cells or a dose of CD4⁺ T cells and a second composition comprising the other of the dose of CD4⁺ T cells and the CD8⁺ T cells. In some embodiments, the dose comprises a first composition and a second composition, and the first composition and second composition are administered within 48 hours of each other, such as no more than 36 hours of each other or not more than 24 hours of each other. In some embodiments, the first composition and second composition are administered 0 to 12 hours apart, 0 to 6 hours apart or 0 to 2 hours apart. In some embodiments, the initiation of administration of the first composition and the initiation of administration of the second composition are carried out no more than 2 hours, no more than 1 hour, or no more than 30 minutes apart, no more than 15 minutes, no more than 10 minutes or no more than 5 minutes apart. In some embodiments, the initiation and/or completion of administration of the first composition and the completion and/or initiation of administration of the second composition are carried out no more than 2 hours, no more than 1 hour, or no more than 30 minutes apart, no more than 15 minutes, no more than 10 minutes or no more than 5 minutes apart. In some embodiments, the first composition and the second composition are administered less than 2 hours apart.

In some composition, the first composition, e.g., first composition of the dose, comprises CD4⁺ T cells. In some composition, the first composition, e.g., first composition of the dose, comprises CD8⁺ T cells. In some embodiments, the first composition is administered prior to the second composition. In particular embodiments, the CD8+ T cells are administered prior to the CD4+ T cells.

In some embodiments, the dose or composition of cells includes a defined or target ratio of CD4⁺ cells expressing a recombinant receptor (e.g. CAR) to CD8⁺ cells expressing a recombinant receptor (e.g. CAR) and/or of CD4⁺ cells to CD8⁺ cells, which ratio optionally is approximately 1:1 or is between approximately 1:3 and approximately 3:1, such as approximately 1:1. In some aspects, the administration of a composition or dose with the target or desired ratio of different cell populations (such as CD4⁺:CD8⁺ ratio or CAR⁺CD4⁺:CAR⁺CD8⁺ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

A. Indolent Follicular Lymphoma (FL) Grade 1-3A

In some embodiments, the provided methods involve treating a specific group or subset of subjects that have follicular lymphoma (FL). In some embodiments, the subject has FL that is a Grade 1-3A, including subjects with relapsed/refractory FL G1-3A. In some embodiments, the provided methods involve a subject having a disease that is an indolent follicular lymphoma (FL). In some embodiments the subject has a disease that is FL Grade 1. In some embodiments, the subject has a disease that is FL Grade 2. In some embodiments, the subject has a disease that is FL Grade 3A.

In particular aspects, the method provided herein include administering to the subject a dose of CD4+ and CD8+ T cells, wherein T cells of each dose comprises a chimeric antigen receptor (CAR) that specifically binds to CD19. Such CAR-T cells, and methods for their manufacture, are described in Section II.

In some embodiments, the provided methods include administration of from or from about 2.5×10⁷ to at or about 1.5×10⁸, such as from about 5×10⁷ to at or about 1×10⁸ total recombinant receptor-expressing T cells (e.g. CAR+ T cells), such as a dose of T cells including CD4+ and CD8+ T cells administered at a defined ratio as described herein, e.g. at or about a 1:1 ratio. In some embodiments, the dose of T cells includes a dose between at or about 5×10⁷ CAR-expressing T cells and at or about 1.5×10⁸ CAR-expressing T cells, inclusive. In some embodiments, the dose of T cells includes a dose between at or about 5×10⁷ CAR-expressing T cells and at or about 1.4×10⁸ CAR-expressing T cells, inclusive. In some embodiments, the dose of T cells includes a dose between at or about 5×10⁷ CAR-expressing T cells and at or about 1.3×10⁸ CAR-expressing T cells, inclusive. In some embodiments, the dose of T cells includes a dose between at or about 5×10⁷ CAR-expressing T cells and at or about 1.2×10⁸ CAR-expressing T cells, inclusive. In some embodiments, the dose of T cells includes a dose between at or about 5×10⁷ CAR-expressing T cells and at or about 1.1×10⁸ CAR-expressing T cells, inclusive. In some embodiments, the dose of T cells includes a dose between at or about 5×10⁷ CAR-expressing T cells and at or about 1.0×10⁸ CAR-expressing T cells, inclusive. In some embodiments, the dose of T cells includes a dose between at or about 5×10⁷ CAR-expressing T cells and at or about 9×10⁷ CAR-expressing T cells, inclusive. In some embodiments, the dose of T cells includes a dose between at or about 5×10⁷ CAR-expressing T cells and at or about 8×10⁷ CAR-expressing T cells, inclusive. In some embodiments, the dose of T cells includes a dose between at or about 5×10⁷ CAR-expressing T cells and at or about 7×10⁷ CAR-expressing T cells, inclusive. In some embodiments, the dose of T cells includes a dose between at or about 5×10⁷ CAR-expressing T cells and at or about 6×10⁷ CAR-expressing T cells, inclusive. In some embodiments, the dose of T cells comprises a ratio of approximately 1:1 CD4+ T cells expressing the CAR to CD8+ T cells expressing the CAR. In particular embodiments, the dose administered is a precise or flat or fixed number of CAR+ T cells, or precise or flat or fixed number of a particular type of CAR+ T cells such as CD4+CAR+ T cells and/or CD8+CAR+ T cells, and/or a number of any of such cells that is within a specified degree of variance, such as no more than, + or − (plus or minus, in some cases indicated as ±), 5, 6, 7, 8, 9, 10, 11, 12, 13, 14, or 15% as compared to such precise or flat or fixed number. In some embodiments, such flat or fixed number of cells is at or about 2.5×10⁷ total CAR+ T cells or of CD8+ and/or CD4+ CAR+ T cells, 5×10⁷ total CAR+ T cells or of CD8+ and/or CD4+ CAR+ T cells, or 1×10⁸ total CAR+ T cells or of CD8+ and/or CD4+ CAR+ T cells.

In some embodiments, the cells of the dose consist of or consist essentially of CAR+ T cells. In some embodiments, the cells of the dose consist of or consist essentially of CD3+ CAR+ T cells. In some embodiments, the cells of the dose consist of or consist essentially of CD4+CAR+ T cells and CD8+CAR+ T cells. In some embodiments, the cells of the dose are free or are essentially free of CD3-cells. In some embodiments, the cells of the dose are free or are essentially free of CAR-cells. In some embodiments, the cells of the dose are free or are essentially free of CD3-CAR-cells.

In some embodiments, the number of cells in the dose includes or consists of or consists essentially of 2.5×10⁷ CAR+ T cells, such as 1.25×10⁷ CD4+CAR+ T cells and 1.25×10⁷ CD8+CAR+ T cells). In some embodiments, the dose includes or consists of or consists essentially of 5×10⁷ CAR+ T cells, such as 2.5×10⁷ CD4+CAR+ T cells and 2.5×10⁷ CD8+CAR+ T cells. In some embodiments, the dose includes or consists or consists essentially of 1×10⁸ CAR+ T cells, such as 0.5×10⁸ CD4+CAR+ T cells and 0.5×10⁸ CD8+CAR+ T cells. In some aspects, the number of cells administered, is within a certain degree of variance of such numbers in the aforementioned embodiments, such as within plus or minus (±) 5, 6, 7, 8, 9, or 10%, such as within plus or minus 8%, as compared to such number(s) of cells. In some aspects, the dose is within a range in which a correlation is observed (optionally a linear relationship) between the number of such cells (e.g., of total CAR+ T cells or of CD8+ and/or CD4+ CAR+ T cells) and one or more outcomes indicative of therapeutic response, or duration thereof (e.g., likelihood of achieving a remission, a complete remission, and/or a particular duration of remission) and/or duration of any of the foregoing.

In some embodiments, the administration comprises administering a plurality of separate compositions, wherein the plurality of separate compositions comprises a first composition comprising the CD8+ CAR-expressing T cells and a second composition comprising the CD4+ CAR-expressing T cells, in accord with a dose in any of the aforementioned embodiments.

In some embodiments, the subject is selected for treatment if the subject has follicular lymphoma (FL) Grade 1-3A. In some embodiments, the subject is selected for treatment if there is PET-positive disease with at least one PET-positive lesion and at least one measurable nodal or extranodal lesion in two perpendicular dimensions. In some embodiments, nodal lesions greater than 1.5 cm in the long axis. In some embodiments, extranodal lesions are >1.0 cm in the long and short axis. In some embodiments, the FL exhibits or is associated with neoplastic follicles that show attenuated mantle zones, loss of polarization, and/or absence of tangible body macrophages. In some embodiments, the FL is associated with a mixture of centrocytes and centroblasts. In some embodiments, the FL is not associated with centrocytes.

In some embodiments, the FL involves lymph nodes and/or spleen, bone marrow, peripheral blood, and other extranodal sites. In some embodiments, the FL involves lymph nodes. In some aspects, exemplary features associated with FL include those described in Choi et al. (2018) Arch Pathol Lab Med 142:1330-1340; Luminari et al., (2012) Rev. Brad. Hematol. Hemoter., 34:54-59 and Salles (2007) ASH Education Book, 2007:216-25. In some aspects, in the case of FL, exemplary parameters used to assess the extent of disease burden include such parameters as hemoglobin levels (e.g., <12 g/dL or <10 g/dL), erythrocyte sedimentation rate (ESR), lactic dehydrogenase (LDH) level, and β2-microglubilin (B2M) value, gene expression, single nucleotide polymorphisms (SNPs; e.g. in IL-8, IL-2, Il-12B, and IL1RN), miRNA expression, and protein expression (e.g., CD68, STAT1, FOXP3, CD57). (Salles (2007) ASH Education Book, 2007:216-25). In the case of FL, the extent or burden of disease may be assessed by the Ann Arbor staging system, tumor burden, bulky disease, number of nodal or extranodal sites of disease, and/or bone marrow involvement.

In some embodiments, the FL is Grade 1. In some embodiments, the Grade 1 FL exhibits or is associated with more than 5 centroblasts per high-powered field (HPF). In some embodiments, the FL is Grade 2. In some embodiments, the Grade 2 FL exhibits or is associated with more than 6 and fewer than 15 centroblasts per high-powered field (HPF). In some embodiments, the FL is Grade 3A. In some embodiments, the Grade 3A FL exhibits or is associated with more than 15 centroblasts per high-powered field (HPF). In some embodiments, the FL is associated with co-expression of CD10, BCL6 and BCL2 within the follicles. In some embodiments, the FL is associated with or characterized by t(14; 18)/IGH-BCL2 and/or BCL6 rearrangements. In some embodiments, the FL is associated with a t(14; 18)(q32; q21) translocation. In some aspects, the t(14; 18)(q32; q21) translocation places BCL2 expression under the control of the immunoglobulin (Ig) heavy locus (IGH) enhancer. In some aspects, t(14; 18) is detected in approximately 90% of grades 1 and 2 FLs and 60 to 70% of Grade 3.

Due to the heterogeneity of the clinical course of patients with FL, one of the challenges of treating FL is the identification of high-risk patients in need of alternate treatment options. Several patient factors have been identified that are associated with higher-risk disease. After initial treatment of newly diagnosed patients with a chemoimmunotherapeutic regimen such as R-CHOP, approximately 25% of patients will progress within 24 months (Casulo et al. Blood. 2015; 125(1):40-7). Patients with progression of disease within 24 months (POD24) have a 5-year overall survival (OS) of 50%, whereas non-POD24 patients have a 5-year OS of 90%. POD24 is an important prognostic survival factor (Casulo, Br J Haematol. 2019; 186(4):513-23). Additional 2L populations with unmet need include patients who are refractory to some of the mainstay FL treatments, i.e., anti-CD20 antibodies and/or alkylating agent. Particularly high-risk patients include those that are double refractory to both an anti-CD20 antibody and an alkylating agent. Since rituximab-based therapies are a standard of care, patients who are refractory to or who relapse early after anti-CD20 therapy have poor outcomes. Time to progression and prior therapies received (e.g., combination therapies, HSCT) are also elements in defining risk of treatment failure in FL. The provided methods include treatment of subject who are high-risk patients. In some embodiments of the provided methods, high risk indolent FL Grade 1-3A patient groups in 2L and subsequent lines of therapy include POD24, rituximab refractory, and the double refractory populations.

In some embodiments, the subject has relapsed or is refractory to treatment after one or more other prior therapy, such as at least two prior therapies, at least three prior therapies or at least four prior therapies. In some embodiments, the subject has relapsed or is refractory to treatment after one or more other prior therapy, such as one prior therapy, two prior therapies, three prior therapies or four prior therapies. Considerations and indications for treatment of relapsed/refractory (r/r) or progressive disease include, among other factors, the modified Groupe d'Etude des Lymphomes Folliculaires (GELF) criteria, which includes: symptoms attributable to FL (not limited to B symptoms); threatened end-organ function; cytopenia secondary to lymphoma; bulky disease (single mass >7 cm or 3 or more masses >3 cm); splenomegaly; and steady progression over at least 6 months (National Comprehensive Cancer Network (NCCN) (NCCN, 2019).

In some embodiments, the subject has relapsed after hematopoietic stem cell transplant. In some embodiments, the transplant is allogeneic or autologous HSCT.

In some embodiments, the subject is refractory to treatment with, or has relapsed during or up to 6 months after completing at least one prior line of therapy that is a chemoimmunotherapeutic combination therapy for treating the disease. In some embodiments, the chemoimmunotherapeutic combination therapy is independently selected from treatment with rituximab; obinutuzumab; bendamustine plus rituximab (BR); bendamustine plus obinutuzumab (BO); R-CHOP; rituximab, cyclophosphamide, vincristine, and prednisone (R-CVP); lenalidomide in combination with rituximab; or a PI3K inhibitor, such as wherein the PI3K inhibitor is idelalisib, copanlisib, or duvelisib. In some embodiments, the prior line of therapy is a chemoimmunotherapeutic combination therapy includes an anti-CD20 monoclonal antibody and an alkylating agent.

In some embodiments the subject has relapsed or is refractory to treatment after at least one prior line of therapy for treating the disease, wherein at least one of the at least one prior lines of therapy includes treatment with an anti-CD20 antibody and an alkylating agent. In some embodiments the subject has relapsed or is refractory to treatment after one prior line of therapy for treating the disease, wherein the one prior lines of therapy includes treatment with an anti-CD20 antibody and an alkylating agent. In some embodiments, the subject the subject has relapsed or is refractory to treatment after at least two prior lines of therapy for treating the disease, wherein at least one of the at least two prior lines of therapy includes treatment with an anti-CD20 antibody and an alkylating agent. In some embodiments, the subject the subject has relapsed or is refractory to treatment after two prior lines of therapy for treating the disease, wherein at least one of the two prior lines of therapy includes treatment with an anti-CD20 antibody and an alkylating agent. In some embodiments, the subject the subject has relapsed or is refractory to treatment after at least three prior lines of therapy for treating the disease, wherein at least one of the at least three prior lines of therapy includes treatment with an anti-CD20 antibody and an alkylating agent. In some embodiments, the subject the subject has relapsed or is refractory to treatment after three prior lines of therapy for treating the disease, wherein at least one of the three prior lines of therapy includes treatment with an anti-CD20 antibody and an alkylating agent.

In some embodiments, the subject is refractory to treatment with, or relapsed during or up to 6 months after completing treatment with, an anti-CD20 therapy and an alkylating agent. In some embodiments, the subject is refractory to treatment with, or relapsed during or up to 6 months after completing a prior line of therapy that includes an anti-CD20 therapy and an alkylating agent. In some embodiments, the subject is refractory to treatment with, or relapsed during or up to 6 months after completing the prior line of therapy that includes anti-CD20 therapy and an alkylating agent. In some embodiments, the subject is refractory to treatment with, or has relapsed during or up to 6 months after completing treatment with, an anti-CD20 therapy. In some embodiments, the subject is administered an anti-CD20 antibody maintenance therapy following completion of a line of therapy. In some embodiments, the subject has relapsed during an anti-CD20 antibody maintenance following 2 or more lines of therapy or within 6 months after maintenance completion. In some embodiments, the subject has relapsed during an anti-CD20 antibody maintenance following the preceding line of therapy or within 6 months after maintenance completion.

Exemplary anti-CD20 antibodies in the aforementioned embodiments can include but are not limited to rituximab, ofatumumab, ocrelizumab (also known as GA101 or RO5072759), veltuzumab, obinutuzumab, TRU-015 (Trubion Pharmaceuticals), ocaratuzumab (also known as AME-133v or ocaratuzumab), and Pro131921 (Genentech). In some embodiments, the anti-CD20 antibody is a monoclonal antibody. In some embodiments, the anti-CD20 antibody comprises rituximab. In some embodiments, the anti-CD20 antibody comprises obinutuzumab. Exemplary alkylating agents in the aforementioned embodiments include but are not limited to cyclophosphamide, decarbazine, melphalan, ifosfamide, temozolomide, and bendamustine. In some embodiments, the alkylating agent is bendamustine.

In some embodiments, the subject had progression of disease within 24 months of diagnosis and after completing a prior line of therapy, such as a chemoimmunotherapeutic regimen. After initial treatment of newly diagnosed patients with a chemoimmunotherapeutic regimen such as R-CHOP, approximately 25% of patients will progress within 24 months (Casulo, 2015). Patients with progression of disease within 24 months (POD24) have a 5-year overall survival (OS) of 50%, whereas non-POD24 patients have a 5-year OS of 90%. POD24 is an important prognostic survival factor (Casulo, Br J Haematol. 2019; 186(4):513-23). In some embodiments, the subject had progression of the disease within 24 months of initial treatment after completing a chemoimmunotherapeutic combination therapy for treating the disease (POD24). In some embodiments, the subject had progression of the disease within 24 months of diagnosis after completing a chemoimmunotherapeutic combination therapy for treating the disease (POD24).

In some embodiments, a cohort of FL subjects is treated according to any of the methods provided herein. In some embodiments, a FL cohort comprises subjects that have relapsed or are refractory to one prior line of therapy for treating FL Grade 1, 2 or 3A, wherein the one prior line of therapy includes treatment with an anti-CD20 antibody and an alkylating agent. In some embodiments, the FL cohort comprises subjects having (i) progressive disease within 24 months of diagnosis of FL (POD24), within 24 months of initiation of the one prior line of therapy (POD24), or both, and (ii) received the one prior line of therapy within six months of the subject's original FL diagnosis. In some embodiments, the FL cohort comprises subjects that have one or more of the following: (i) symptoms attributable to; (ii) threatened end-organ function, cytopenia secondary to lymphoma, or bulky disease ; (iii) splenomegaly; and (iv) steady progression of disease over at least 6 months. Thus in some embodiments, the FL cohort comprises subjects that have relapsed or are refractory to one prior line of therapy for treating FL Grade 1, 2 or 3A, wherein the one prior line of therapy includes treatment with an anti-CD20 antibody and an alkylating agent; and (i) has progressive disease within 24 months of diagnosis of FL (POD24), within 24 months of initiation of the one prior line of therapy (POD24), or both, and received the one prior line of therapy within six months of the subject's original FL diagnosis; or (ii) has one or more of symptoms attributable to FL; threatened end-organ function, cytopenia secondary to lymphoma, or bulky disease; splenomegaly; and steady progression of disease over at least 6 months. In some embodiments, the symptoms attributable to FL are not limited to B symptoms. In some embodiments, bulky disease is a single mass >7 cm or 3 or more masses >3 cm.

In some embodiments, a cohort of FL subjects is treated according to any of the methods provided herein. In some embodiments, a FL cohort comprises subjects that have relapsed or are refractory to two prior lines of therapy for treating FL Grade 1, 2 or 3A, wherein one of the two prior lines of therapy includes treatment with an anti-CD20 antibody and an alkylating agent. In some embodiments, the FL cohort comprises subjects that (i) have relapsed or refractory disease within 12 months of completion of a prior line of therapy (e.g. the first prior line of therapy or the second prior line of therapy) and have received prior combination therapies; (ii) have relapsed after HSCT, and/or (iii) meet the definition of double refractory. Thus, in some embodiments, the FL cohort comprises subjects (i) that have relapsed or are refractory to two prior lines of therapy for treating FL Grade 1, 2 or 3A, wherein one of the two prior lines of therapy includes treatment with an anti-CD20 antibody and an alkylating agent; and (ii) that have relapsed or refractory disease within 12 months of completion of a prior line of therapy (e.g. the first prior line of therapy or the second prior line of therapy) and have received prior combination therapies; have relapsed after HSCT, and/or meet the definition of double refractory. In some embodiments, monotherapy with a PI3Ki is a prior line of therapy. In some embodiments, a subject is double refractory if the subject (i) is refractory a systemic line of therapy including an anti-CD20 antibody and an alkylating agent; (ii) has relapsed within six months after completion of a prior line of systemic therapy including an anti-CD20 antibody and an alkylating agent; and/or (iii) has relapsed during anti-CD20 antibody maintenance therapy provided after the two prior lines or therapy or within 6 months of completing anti-CD20 antibody maintenance therapy provided after the two prior lines or therapy.

In some embodiments a cohort of FL subjects is treated according to any of the methods provided herein. In some embodiments, a FL cohort comprises subjects that have relapsed or are refractory to three or more (e.g. three) prior lines of therapy for treating FL Grade 1, 2 or 3A, wherein one of the three or more prior lines of therapy includes treatment with an anti-CD20 antibody and an alkylating agent. In some embodiments, HSCT is one of the three or more prior lines of therapy. In some embodiments, a FL cohort comprises subjects that are double refractory. In some embodiments, a subject is double refractory if the subject (i) is refractory a systemic line of therapy including an anti-CD20 antibody and an alkylating agent; (ii) has relapsed within six months after completion of a prior line of systemic therapy including an anti-CD20 antibody and an alkylating agent; and/or (iii) has relapsed during anti-CD20 antibody maintenance therapy provided after the two prior lines or therapy or within 6 months of completing anti-CD20 antibody maintenance therapy provided after the two prior lines or therapy.

In some embodiments, the subject does not have FL grade 3B (FL3B). In some embodiments, the subject does not have evidence of composite DLBCL and FL, or of transformed FL. In some embodiments, the subject does not have World Health Organization (WHO) subclassification of duodenal-type FL

B. Marginal Zone Lymphoma (MZL)

In some embodiments, the provided methods involve treating a specific group or subset of subjects that have marginal zone lymphoma (MZL). In some embodiments, the subject has relapsed/refractory MZL.

MZL is a heterogeneous B-cell malignancy that derives from transformation of B-cells that mature in the secondary lymphoid follicles before translocating to the marginal zones of mucosa associated lymphoid tissues (MALT), the spleen, or the lymph nodes. In some embodiments, the MZL exhibits or is associated with marginal zone lymphocytes characterized by hypermutated IgV genes. In some embodiments, the marginal zone lymphocytes exhibit or are associated with a pan-B+; CD5−/+; CD10−; CD23−; CD11c+/−; cyIg +(40% of the cells), sIgM+ bright; sIgD-immunophenotype.

Three subtypes are described: extra-nodal MZL (ENMZL, mostly gastric), splenic MZL (SMZL), and nodal MZL (NMZL). In some aspects, the subject has extranodal MZL. In some aspects, extranodal MZL can be detected in the MALT of the gastrointestinal tract, thyroid gland, breast, lung, spleen and/or other MALT sites. In some embodiments, extranodal MZL can be detected in the stomach. In some aspects, the extra-nodal MZL exhibits or is associated with the translocation t(11:18)(q21; q21)/API2/MLT fusion. In some aspects, the extra-nodal MZL exhibits or is associated with the translocation t(14:18)(q32; q21)/IgH/MLT1 fusion. In some aspects, the extra-nodal MZL exhibits or is associated with the t(3:14)/IgH/FOXP1 fusion. In some embodiments, the subject has splenic MZL wherein disease is initially only detected in the spleen, bone marrow, and/or blood. In some embodiments, splenic MZL exhibits or is associated with splenomegaly associated circulating villous lymphocytes. In some aspects, the splenic MZL exhibits or is associated with 7q deletions. In some aspects, the deletion spans a region between 7q32.1 to 7q32-3. In some aspects, the splenic MZL exhibits or is associated with 7q translocations. In some aspects, the translocation is centered around the 7q22-q32 region. In some aspects, the splenic MZL exhibits or is associated with trisomy 3. In some aspects, the splenic MZL exhibits or is associated with trisomy 12. In some embodiments, the subject has nodal MZL, wherein the lymphoma is first restricted to the lymph nodes, bone marrow, and/or blood (Swerdlow et al., (2016) Blood, 30:84-91).

In particular aspects, the method provided herein include administering to the subject a dose of CD4+ and CD8+ T cells, wherein T cells of each dose comprises a chimeric antigen receptor (CAR) that specifically binds to CD19. Such CAR-T cells, and methods for their manufacture, are described in Section II.

In some embodiments, the provided methods include administration of from or from about 2.5×10⁷ to at or about 1.5×10⁸, such as from about 5×10⁷ to at or about 1×10⁸ total recombinant receptor-expressing T cells (e.g. CAR+ T cells), such as a dose of T cells including CD4+ and CD8+ T cells administered at a defined ratio as described herein, e.g. at or about a 1:1 ratio. In some embodiments, the dose of T cells includes a dose between at or about 5×10⁷ CAR-expressing T cells and at or about 1.5×10⁸ CAR-expressing T cells, inclusive. In some embodiments, the dose of T cells includes a dose between at or about 5×10⁷ CAR-expressing T cells and at or about 1.1×10⁸ CAR-expressing T cells, inclusive. In some embodiments, the dose of T cells includes a dose between at or about 5×10⁷ CAR-expressing T cells and at or about 1.0×10⁸ CAR-expressing T cells, inclusive. In some embodiments, the dose of T cells includes a dose between at or about 5×10⁷ CAR-expressing T cells and at or about 9×10⁷ CAR-expressing T cells, inclusive. In some embodiments, the dose of T cells includes a dose between at or about 5×10⁷ CAR-expressing T cells and at or about 8×10⁷ CAR-expressing T cells, inclusive. In some embodiments, the dose of T cells includes a dose between at or about 5×10⁷ CAR-expressing T cells and at or about 7×10⁷ CAR-expressing T cells, inclusive. In some embodiments, the dose of T cells includes a dose between at or about 5×10⁷ CAR-expressing T cells and at or about 6×10⁷ CAR-expressing T cells, inclusive. In some embodiments, the dose of T cells comprises a ratio of approximately 1:1 CD4+ T cells expressing the CAR to CD8+ T cells expressing the CAR. In particular embodiments, the dose administered is a precise or flat or fixed number of CAR+ T cells, or precise or flat or fixed number of a particular type of CAR+ T cells such as CD4+CAR+ T cells and/or CD8+CAR+ T cells, and/or a number of any of such cells that is within a specified degree of variance, such as no more than, + or − (plus or minus, in some cases indicated as ±), 5, 6, 7, 8, 9, 10, 11, 12, 13, 14, or 15% as compared to such precise or flat or fixed number. In some embodiments, such flat or fixed number of cells is at or about 2.5×10⁷ total CAR+ T cells or of CD8+ and/or CD4+ CAR+ T cells, 5×10⁷ total CAR+ T cells or of CD8+ and/or CD4+ CAR+ T cells, or 1×10⁸ total CAR+ T cells or of CD8+ and/or CD4+ CAR+ T cells. In some embodiments, the number of cells in the dose includes or consists of or consists essentially of 2.5×10⁷ CAR+ T cells, such as 1.25×10⁷ CD4+CAR+ T cells and 1.25×10⁷ CD8+CAR+ T cells). In some embodiments, the dose includes or consists of or consists essentially of 5×10⁷ CAR+ T cells, such as 2.5×10⁷ CD4+CAR+ T cells and 2.5×10⁷ CD8+CAR+ T cells. In some embodiments, the dose includes or consists or consists essentially of 1×10⁸ CAR+ T cells, such as 0.5×10⁸ CD4+CAR+ T cells and 0.5×10⁸ CD8+CAR+ T cells. In some aspects, the number of cells administered, is within a certain degree of variance of such numbers in the aforementioned embodiments, such as within plus or minus (±) 5, 6, 7, 8, 9, or 10%, such as within plus or minus 8%, as compared to such number(s) of cells. In some aspects, the dose is within a range in which a correlation is observed (optionally a linear relationship) between the number of such cells (e.g., of total CAR+ T cells or of CD8+ and/or CD4+ CAR+ T cells) and one or more outcomes indicative of therapeutic response, or duration thereof (e.g., likelihood of achieving a remission, a complete remission, and/or a particular duration of remission) and/or duration of any of the foregoing.

In some embodiments, the administration comprises administering a plurality of separate compositions, wherein the plurality of separate compositions comprises a first composition comprising the CD8+ CAR-expressing T cells and a second composition comprising the CD4+ CAR-expressing T cells, in accord with a dose in any of the aforementioned embodiments.

In some embodiments, the subject is selected for treatment if the subject has a MZL. In some embodiments, the subject is selected for treatment if there is histologically confirmed disease. In some embodiments, the subject is selected for treatment if there is CT confirmed disease. In some embodiments, the subject is selected for treatment if there is PET confirmed disease. In some embodiments, the subject is selected for treatment if there is PET non-avid disease. In some embodiments, the subject is selected for treatment if the subject has at least one measurable nodal lesion greater than 2.0 cm in the long axis. In some embodiments, the patient has at least one measurable nodal lesion. In some embodiments, the patient has at least one measurable extranodal lesion.

In some embodiments, the subject has relapsed or is refractory to treatment after at least two prior lines of therapy for treating MZL. In some embodiments, the subject has relapsed or is refractory to treatment after two prior lines of therapy for treating MZL. The prior line(s) of therapy can include any therapy that is used for treating MZL. In some embodiments, one or more of the prior lines of therapy is selected from treatment with a CD20-targeted therapy (e.g. anti-CD20 antibody, such as rituximab or obinutuzumab), ibrutinib, or a hematopoietic stem cell transplant (HSCT). In some cases, the HSCT is allogeneic. In some cases, the HSCT is autologous.

In particular embodiments, the subject has received prior line of therapy that is a combination chemoimmunotherapeutic therapy that includes a CD20-targeted therapy (e.g. anti-CD20 antibody). In some embodiments, the combination is a combination systemic therapy. Among subjects treated are subjects that have relapsed or is refractory to treatment with a combination systemic therapy for treating MZL. In some embodiments, the combination systemic therapy includes an anti-CD20 antibody and an alkylating agent. In some embodiments, the anti-CD20 antibody is a monoclonal antibody. In some embodiments, the anti-CD20 antibody is rituximab. In some embodiments, the anti-CD20 antibody is obinutuzumab. Exemplary alkylating agents include but are not limited to cyclophosphamide, chlorambucil, decarbazine, melphalan, ifosfamide, temozolomide, and bendamustine. In some embodiments, the alkylating agent is bendamustine In some embodiments, the alkylating agent is chlorambucil. In some embodiments, the subject has been previously treated with rituximab and bendamustine. In some embodiments, the subject has been previously treated with rituximab and chlorambucil. In some embodiments, the subject has been previously treated with obinutuzumab and bendamustine. In some embodiments, the subject has been previously treated with obinutuzumab and chlorambucil.

In some embodiments, a prior line of therapy is rituximab plus cyclophosphamide, doxorubicin, vincristine, and prednisone (R-CHOP). In some embodiments, the subject has relapsed or is refractory to treatment with rituximab plus cyclophosphamide, doxorubicin, vincristine, and prednisone (R-CHOP). In some embodiments, the subject has relapsed or is refractory to treatment with lenalidomide and rituximab.

In some embodiments, a prior line of therapy is hematopoietic stem cell transplant (HSCT). In some embodiments, the subject has relapsed or is refractory to a hematopoietic stem cell transplant (HSCT). In some embodiments, the subject had previously received, and relapsed or is refractory to, an allogeneic HSCT. In some embodiments, the subject had previously received, and relapsed or is refractory to, an autologous HSCT.

In some embodiments a cohort of MZL subjects is treated according to any of the methods provided herein. In some embodiments, a MZL cohort comprises subjects that have relapsed or are refractory to two prior lines of therapy for treating MZL, wherein at least one line of prior therapy was a combination systemic therapy, therapy with an anti-CD20 antibody and an alkylating agent, or HSCT. In some embodiments, the subject is relapsed after HSCT. In some embodiments, the MZL is splenic MZL and one of the two prior lines of therapy is a splenectomy. In some embodiments, the MZL is extranodal MZL and antibiotics are not one of the two prior lines of therapy.

In some embodiments, the subject has splenic MZL and at least one of the at least two prior therapies is a splenectomy. In some embodiments, the subject has splenic MZL and at least one of the two prior therapies is a splenectomy. In some embodiments, subject has extranodal MZL (ENMZL) and an antibiotic is not one of the at least two prior lines of therapy. In some embodiments, subject has extranodal MZL (ENMZL) and an antibiotic is not one of the two prior lines of therapy.

C. Response, Efficacy, and Survival

In some embodiments, the administration in accord with the provided methods effectively treats the subject despite the subject having become resistant to another therapy. In some embodiments, at least 30%, at least 35%, at least 40% at least 50%, at least 60%, at least 70%, or at least 80%, of subjects treated according to the method achieve complete remission (CR). In some embodiments, at least about 40%, at least about 50%, at least about 60%, at least about 70%, at least 80%, or at least 90% of the subjects treated according to the method achieve an objective response (OR). In some embodiments, at least or at least about 50% of subjects, at least or at least about 60% of the subjects, at least or at least about 70% of the subjects, at least or at least about 80% of the subjects or at least or at least about 90% of the subjects treated according to the method achieve CR and/or achieve an objective response (OR). In some embodiments, criteria assessed for effective treatment includes overall response rate (ORR; also known in some cases as objective response rate), complete response (CR; also known in some cases as complete remission), duration of response (DOR), progression-free survival (PFS), and/or overall survival (OS).

In some embodiments, at least 40%, at least 50%, at least 60%, or at least 70% of subjects treated according to the methods provided herein achieve complete remission (CR; also known in some cases as complete response), exhibit progression-free survival (PFS) and/or overall survival (OS) for greater than at or about 3 months, 6 months or 12 months or greater than 13 months or approximately 14 months. In some embodiments, on average, subjects treated according to the method exhibit a median PFS or OS of greater than at or about 6 months, 12 months, or 18 months. In some embodiments, the subject exhibits PFS or OS following therapy for at least at or about 6, 12, 18 or more months or longer.

In some embodiments, the subjects treated according to the provided methods exhibits a CRR of at least 50%, at least 60%, at least 70%, at least 80%, or at least 90%. In some embodiments, the complete response rate (CRR) is calculated as the percentage of subjects with the best overall response (BOR) up to 12 months, up to 18 months, up to 24 months, up to 36 months or longer.

In some aspects, response rates in subjects, such as subjects with NHL, are based on the Lugano criteria. (Cheson et al., Blood. 2016; 128(21):2489-96.). In some aspects, response assessment utilizes any of clinical, hematologic, and/or molecular methods. In some aspects, response assessed using the Lugano criteria involves the use of positron emission tomography (PET)-computed tomography (CT) and/or CT as appropriate. PET-CT evaluations may further comprise the use of fluorodeoxyglucose (FDG) for FDG-avid lymphomas. In some aspects, where PET-CT will be used to assess response in FDG-avid histologies, a 5-point scale may be used. In some respects, the 5-point scale comprises the following criteria: 1, no uptake above background; 2, uptake ≤mediastinum; 3, uptake >mediastinum but ≤liver; 4, uptake moderately >liver; 5, uptake markedly higher than liver and/or new lesions; X, new areas of uptake unlikely to be related to lymphoma.

In some aspects, a complete response as described using the Lugano criteria involves a complete metabolic response and a complete radiologic response at various measurable sites. In some aspects, these sites include lymph nodes and extralymphatic sites, wherein a CR is described as a score of 1, 2, or 3 with or without a residual mass on the 5-point scale, when PET-CT is used. In some aspects, extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake.

In some aspects, response is assessed in the lymph nodes using CT, wherein a CR is described as no extralymphatic sites of disease and target nodes/nodal masses must regress to ≤1.5 cm in longest transverse diameter of a lesion (LDi). Further sites of assessment include the bone marrow wherein PET-CT-based assessment should indicate a lack of evidence of FDG-avid disease in marrow and a CT-based assessment should indicate a normal morphology. Further sites may include assessment of organ enlargement, which should regress to normal. In some aspects, non-measured lesions and new lesions are assessed, which in the case of CR should be absent (Chessen et al., Blood. 2016 Nov. 24; 128(21):2489-96).

In some aspects, a partial response (PR; also known in some cases as partial remission) as described using the Lugano criteria involves a partial metabolic and/or radiological response at various measurable sites. In some aspects, these sites include lymph nodes and extralymphatic sites, wherein a PR is described as a score of 4 or 5 with reduced uptake compared with baseline and residual mass(es) of any size, when PET-CT is used. At interim, such findings can indicate responding disease. At the end of treatment, such findings can indicate residual disease.

In some aspects, response is assessed in the lymph nodes using CT, wherein a PR is described as ≥50% decrease in SPD of up to 6 target measurable nodes and extranodal sites. If a lesion is too small to measure on CT, 5 mm×5 mm is assigned as the default value; if the lesion is no longer visible, the value is 0 mm×0 mm; for a node >5 mm×5 mm, but smaller than normal, actual measurements are used for calculation. Further sites of assessment include the bone marrow wherein PET-CT-based assessment should indicate residual uptake higher than uptake in normal marrow but reduced compared with baseline (diffuse uptake compatible with reactive changes from chemotherapy allowed). In some aspects, if there are persistent focal changes in the marrow in the context of a nodal response, consideration should be given to further evaluation with MRI or biopsy, or an interval scan. In some aspects, further sites may include assessment of organ enlargement, where the spleen must have regressed by >50% in length beyond normal. In some aspects, non-measured lesions and new lesions are assessed, which in the case of PR should be absent/normal, regressed, but no increase. No response/stable disease (SD) or progressive disease (PD) can also be measured using PET-CT and/or CT based assessments. (Chessen et al., Blood. 2016 Nov. 24; 128(21):2489-96).

In some respects, progression-free survival (PFS) is described as the length of time during and after the treatment of a disease, such as cancer, that a subject lives with the disease but it does not get worse. In some aspects, objective response (OR) is described as a measurable response. In some aspects, objective response rate (ORR; also known in some cases as overall response rate) is described as the proportion of patients who achieved CR or PR. In some aspects, overall survival (OS) is described as the length of time from either the date of diagnosis or the start of treatment for a disease, such as cancer, that subjects diagnosed with the disease are still alive. In some aspects, event-free survival (EFS) is described as the length of time after treatment for a cancer ends that the subject remains free of certain complications or events that the treatment was intended to prevent or delay. These events may include the return of the cancer or the onset of certain symptoms, such as bone pain from cancer that has spread to the bone, or death.

In some embodiments, the measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response is durable for greater than 3 months or greater than 6 months.

In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative dosing regimen, such as one in which the subject receives one or more alternative therapeutic agents and/or one in which the subject does not receive a dose of cells and/or a lymphodepleting agent in accord with the provided methods, and/or with the provided articles of manufacture or compositions. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified.

In some embodiments, the event-free survival rate or overall survival rate of the subject is improved by the methods, as compared with other methods, for example, methods in which the subject receives one or more alternative therapeutic agents and/or one in which the subject does not receive a dose of cells and/or a lymphodepleting agent in accord with the provided methods, and/or with the provided articles of manufacture or compositions. For example, in some embodiments, event-free survival rate or probability for subjects treated by the methods at 6 months following the dose is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods, for example, methods in which the subject receives one or more alternative therapeutic agents and/or one in which the subject does not receive a dose of cells and/or a lymphodepleting agent in accord with the provided methods, and/or with the provided articles of manufacture or compositions. For example, in some embodiments, the probability of relapse at 6 months following the first dose is less than about 80%, less than about 70%, less than about 60%, less than about 50%, less than about 40%, less than about 30%, less than about 20%, or less than about 10%.

In some cases, the pharmacokinetics of administered cells, e.g., adoptively transferred cells are determined to assess the availability, e.g., bioavailability of the administered cells. Methods for determining the pharmacokinetics of adoptively transferred cells may include drawing peripheral blood from subjects that have been administered engineered cells, and determining the number or ratio of the engineered cells in the peripheral blood. Approaches for selecting and/or isolating cells may include use of chimeric antigen receptor (CAR)-specific antibodies (e.g., Brentjens et al., Sci. Transl. Med. 2013 March; 5(177): 177ra38) Protein L (Zheng et al., J. Transl. Med. 2012 February; 10:29), epitope tags, such as Strep-Tag sequences, introduced directly into specific sites in the CAR, whereby binding reagents for Strep-Tag are used to directly assess the CAR (Liu et al. (2016) Nature Biotechnology, 34:430; international patent application Pub. No. WO2015095895) and monoclonal antibodies that specifically bind to a CAR polypeptide (see international patent application Pub. No. WO2014190273). Extrinsic marker genes may in some cases be utilized in connection with engineered cell therapies to permit detection or selection of cells and, in some cases, also to promote cell suicide. A truncated epidermal growth factor receptor (EGFRt) in some cases can be co-expressed with a transgene of interest (a CAR or TCR) in transduced cells (see e.g. U.S. Pat. No. 8,802,374). EGFRt may contain an epitope recognized by the antibody cetuximab (Erbitux®) or other therapeutic anti-EGFR antibody or binding molecule, which can be used to identify or select cells that have been engineered with the EGFRt construct and another recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434).

In some embodiments, the number of CAR⁺ T cells in a biological sample obtained from the patient, e.g., blood, can be determined at a period of time after administration of the cell therapy, e.g., to determine the pharmacokinetics of the cells. In some embodiments, number of CAR⁺ T cells, optionally CAR⁺ CD8⁺ T cells and/or CAR⁺ CD4⁺ T cells, detectable in the blood of the subject, or in a majority of subjects so treated by the method, is greater than 1 cells per μL, greater than 5 cells per μL or greater than per 10 cells per μL. In some embodiments, the number of CAR⁺ T cells in a biological sample obtained from the patient, e.g., blood, can be determined via PCR for the CAR transgene.

D. Toxicity

In some embodiments, subjects treated according to any of the provided methods are assessed for one or more signs or symptoms of toxicity that may be associated with the administered cells. Administration of adoptive T cell therapy, such as treatment with T cells expressing chimeric antigen receptors, can induce toxic effects or outcomes such as cytokine release syndrome and neurotoxicity. In some examples, such effects or outcomes parallel high levels of circulating cytokines, which may underlie the observed toxicity.

In some aspects, the toxic outcome is or is associated with or indicative of cytokine release syndrome (CRS) or severe CRS (sCRS). CRS, e.g., sCRS, can occur in some cases following adoptive T cell therapy and administration to subjects of other biological products. See Davila et al., Sci Transl Med 6, 224ra25 (2014); Brentjens et al., Sci. Transl. Med. 5, 177ra38 (2013).

Typically, CRS is caused by an exaggerated systemic immune response mediated by, for example, T cells, B cells, NK cells, monocytes, and/or macrophages. Such cells may release a large amount of inflammatory mediators such as cytokines and chemokines. Cytokines may trigger an acute inflammatory response and/or induce endothelial organ damage, which may result in microvascular leakage, heart failure, or death. Severe, life-threatening CRS can lead to pulmonary infiltration and lung injury, renal failure, or disseminated intravascular coagulation. Other severe, life-threatening toxicities can include cardiac toxicity, respiratory distress, neurologic toxicity and/or hepatic failure. In some aspects, fever, especially high fever (≥38.5° C. or ≥101.3° F.), is associated with CRS or risk thereof. In some cases, features or symptoms of CRS mimic infection. In some embodiments, infection is also considered in subjects presenting with CRS symptoms, and monitoring by cultures and empiric antibiotic therapy can be administered. Other symptoms associated with CRS can include cardiac dysfunction, adult respiratory distress syndrome, renal and/or hepatic failure, coagulopathies, disseminated intravascular coagulation, and capillary leak syndrome.

CRS may be treated using anti-inflammatory therapy such as an anti-IL-6 therapy, e.g., anti-IL-6 antibody, e.g., tocilizumab, or antibiotics or other agents as described. Outcomes, signs and symptoms of CRS are known and include those described herein. In some embodiments, where a particular administration affects or does not affect a given CRS-associated outcome, sign, or symptom, particular outcomes, signs, and symptoms and/or quantities or degrees thereof may be specified.

In the context of administering CAR-expressing cells, CRS typically occurs within two weeks after infusion of cells that express a CAR. See Abramson et al., J Clin Onc. 2018; 36(15_suppl):7505. In some cases, CRS occurs less than 3 days or more than 21 days after CAR T cell infusion. The incidence and timing of CRS may be related to baseline cytokine levels or tumor burden at the time of infusion. Commonly, CRS involves elevated serum levels of interferon (IFN)-γ, tumor necrosis factor (TNF)-α, and/or interleukin (IL)-2. Other cytokines that may be rapidly induced in CRS are IL-1β, IL-6, IL-8, and IL-10.

Exemplary outcomes associated with CRS include fever, rigors, chills, hypotension, dyspnea, acute respiratory distress syndrome (ARDS), encephalopathy, ALT/AST elevation, renal failure, cardiac disorders, hypoxia, neurologic disturbances, and death. Neurological complications include delirium, seizure-like activity, confusion, word-finding difficulty, aphasia, and/or becoming obtunded. Other CRS-related outcomes include fatigue, nausea, headache, seizure, tachycardia, myalgias, rash, acute vascular leak syndrome, liver function impairment, and renal failure. In some aspects, CRS is associated with an increase in one or more factors such as serum-ferritin, d-dimer, aminotransferases, lactate dehydrogenase and triglycerides, or with hypofibrinogenemia or hepatosplenomegaly. Other exemplary signs or symptoms associated with CRS include hemodynamic instability, febrile neutropenia, increase in serum C-reactive protein (CRP), changes in coagulation parameters (for example, international normalized ratio (INR), prothrombin time (PTI) and/or fibrinogen), changes in cardiac and other organ function, and/or absolute neutrophil count (ANC).

In some embodiments, outcomes associated with CRS include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures). In some embodiments, neurotoxicity (NT) can be observed concurrently with CRS.

Exemplary CRS-related outcomes include increased or high serum levels of one or more factors, including cytokines and chemokines and other factors associated with CRS. Exemplary outcomes further include increases in synthesis or secretion of one or more of such factors. Such synthesis or secretion can be by the T cell or a cell that interacts with the T cell, such as an innate immune cell or B cell.

CRS criteria that appear to correlate with the onset of CRS to predict which patients are more likely to be at risk for developing sCRS have been developed (see Davilla et al. Science translational medicine. 2014; 6(224):224ra25; Abramson et al., J Clin Onc. 2018; 36(15_suppl):7505). Factors include fevers, hypoxia, hypotension, neurologic changes, elevated serum levels of inflammatory cytokines, such as a set of seven cytokines (IFNγ, IL-5, IL-6, IL-10, Flt-3L, fractalkine, and GM-CSF) whose treatment-induced elevation can correlate well with both pretreatment tumor burden and sCRS symptoms. In some embodiments, the criteria reflective of CRS grade are those detailed in Table 2 below.

TABLE 2
Grading Criteria for Cytokine Release Syndrome

| | | Cytokine | | | |
|---|---|---|---|---|---|
| | | Release | | | CRS Grade 4 |
| | | Syndrome | CRS Grade 2 | CRS Grade 3 | (life- |
| | | (CRS) | (moderate) | (severe) | threatening) |
| | | Grade 1 | CRS grade is defined by the most severe symptom | | |
| | Symptoms/Signs | (mild) | (excluding fever) | | |
| Vital Signs | Temperature | Yes | Any | Any | Any |
| | >38.5° C./101.3° F. | | | | |
| | Systolic blood pressure | N/A | Responds to | Needs high- | Life- |
| | (SBP) ≤ 90 mmHg | | intravenous (IV) | dose or multiple | threatening |
| | | | fluids or single low- | vasopressors | |
| | | | dose vasopressor | | |
| | Need for oxygen to | N/A | Fraction of inspired | FiO₂ ≥ 40% | Needs |
| | reach oxygen saturation | | oxygen (FiO₂) < 40% | | ventilator |
| | (SaO₂) > 90% | | | | support |
| Organ | | N/A | Grade 2 | Grade 3 or | Grade 4 |
| Toxicity | | | | transaminitis | (excluding |
| | | | | Grade 4 | transaminitis) |

In some embodiments, high-dose vasopressor therapy include those described in Table 3 below.

TABLE 3
High dose vasopressors (all doses required for ≥ 3 hours)

| Vasopressor | Dose |
|---|---|
| Norepinephrine monotherapy | ≥20 μg/min |
| Dopamine monotherapy | ≥10 μg/kg/min |
| Phenylephrine monotherapy | ≥200 μg/min |
| Epinephrine monotherapy | ≥10 μg/min |
| If on vasopressin | Vasopressin + norepinephrine |
| | equivalent (NE) of ≥ 10 μg/min^{a} |
| If on combination vasopressors (not | Norepinephrine equivalent of |
| vasopressin) | ≥20 ug/min^{a} |

^{a}VASST Trial Vasopressor Equivalent Equation: Norepinephrine equivalent dose = [norepinephrine (μg/min)] + [dopamine (μg/kg/min) ÷ 2] + [epinephrine (μg/min)] + [phenylephrine (ug/min) ÷ 10]

In some embodiments, the toxic outcome is a severe CRS. In some embodiments, the toxic outcome is the absence of severe CRS (e.g. moderate or mild CRS).

In some embodiments, fever and/or levels of C-reactive protein (CRP) can be measured. In some embodiments, the CRS-associated serum factors or CRS-related outcomes include an increase in the level and/or concentration of inflammatory cytokines and/or chemokines, including Flt-3L, fracktalkine, granulocyte macrophage colony stimulating factor (GM-CSF), interleukin-1 beta (IL-1β, IL-2, IL-5, IL-6, IL-7, IL-8, IL-10, IL-12, interferon gamma (IFN-γ), macrophage inflammatory protein (MIP)-1, MIP-1, sIL-2Rα, or tumor necrosis factor alpha (TNFα). In some embodiments, the factor or outcome includes C reactive protein (CRP). In some embodiments, subjects that are measured to have high levels of CRP do not have CRS. In some embodiments, a measure of CRS includes a measure of CRP and another factor indicative of CRS.

In some embodiments, outcomes associated with severe CRS or grade 3 CRS or greater, such as grade 4 or greater, include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures). In some embodiments, severe CRS includes CRS that requires management or care in the intensive care unit (ICU).

In some embodiments, the CRS, such as severe CRS, encompasses a combination of (1) persistent fever (fever of at least 38 degrees Celsius for at least three days) and (2) a serum level of CRP of at least at or about 20 mg/dL. In some embodiments, the CRS encompasses hypotension requiring the use of two or more vasopressors or respiratory failure requiring mechanical ventilation. In some embodiments, the dosage of vasopressors is increased in a second or subsequent administration.

In some embodiments, severe CRS or grade 3 CRS encompasses an increase in alanine aminotransferase, an increase in aspartate aminotransferase, chills, febrile neutropenia, headache, left ventricular dysfunction, encephalopathy, hydrocephalus, and/or tremor. In some embodiments, severe CRS is treated with additional T cell depleting therapies such as cyclophosphamide (Brudno et al., Blood. 2016; 127(26):3321-30).

The method of measuring or detecting the various outcomes may be specified.

In some aspects, the toxic outcome is or is associated with neurotoxicity. In some embodiments, symptoms associated with a clinical risk of neurotoxicity include confusion, delirium, aphasia, expressive aphasia, obtundation, myoclonus, lethargy, altered mental status, convulsions, seizure-like activity, seizures (optionally as confirmed by electroencephalogram (EEG)), elevated levels of beta amyloid (Aβ), elevated levels of glutamate, and elevated levels of oxygen radicals. In some embodiments, neurotoxicity is graded based on severity (e.g., using a Grade 1-5 scale (see, e.g., National Cancer Institute—Common Toxicity Criteria version 5.00 (NCI CTCAE version 5.0)

In some instances, neurologic symptoms may be the earliest symptoms of sCRS. In some embodiments, neurologic symptoms are seen to begin 5 to 7 days after cell therapy infusion. In some embodiments, duration of neurologic changes may range from 3 to 23 days. In some cases, recovery of neurologic changes occurs after other symptoms of sCRS have resolved. In some embodiments, time or degree of resolution of neurologic changes is not hastened by treatment with anti-IL-6 and/or steroid(s).

In some embodiments, severe neurotoxicity includes neurotoxicity with a grade of 3 or greater, such as set forth in Table 4.

TABLE 4
Exemplary Grading Criteria for neurotoxicity

| Grade | Description of Symptoms |
|---|---|
| 1 | Transient or mild discomfort; no limitation in activity; no medical |
| Asymptomatic or Mild | intervention/therapy required |
| 2 | Presence of symptoms that limit instrumental activities of daily living (ADL), |
| Moderate | such as preparing meals, shopping for groceries or clothes, using the |
| | telephone, managing money |
| 3 | Presence of symptoms that limit self-care ADL, such as bathing, dressing and |
| Severe | undressing, feeding self, using the toilet, taking medications |
| 4 | Symptoms that are life-threatening, requiring urgent intervention |
| Life-threatening | |
| 5 | Death |
| Fatal | |

In some embodiments, one or more interventions or agents for treating the toxicity, such as a toxicity-targeting therapies, is administered at a time at which or immediately after which the subject is determined to or confirmed to (such as is first determined or confirmed to) exhibit sustained fever, for example, as measured according to any of the aforementioned embodiments. In some embodiments, the one or more toxicity-targeting therapies is administered within a certain period of time of such confirmation or determination, such as within 30 minutes, 1 hour, 2 hours, 3 hours, 4 hours, 6 hours, or 8 hours thereof.

In some embodiments, the resulting response observed in subjects treated in accord with the provided methods, and/or with the provided articles of manufacture or compositions, is associated with or results in a low risk of any toxicity or a low risk of severe toxicity in a majority of the subjects treated. In some embodiments, greater than or greater than about 30%, 35%, 40%, 50%, 55%, 60% , 70%, 80%, or 90% or more of the subjects treated according to the provided methods and/or with the provided articles of manufacture or compositions do not exhibit any grade of CRS or any grade of neurotoxicity (NT). In some embodiments, greater than or greater than about 50%, 60%, 70%, 80%, 90%, 95% or more of the subjects treated according to the provided methods and/or with the provided articles of manufacture or compositions do not exhibit severe CRS or grade 3 or higher CRS. In some embodiments, greater than or greater than about 50%, 60%, 70%, 80%, 90% or 95% or more of the subjects treated according to the provided methods, and/or with the provided articles of manufacture or compositions, do not exhibit severe neurotoxicity or grade 3 or higher neurotoxicity, such as grade 4 or 5 neurotoxicity.

In some embodiments, at least at or about 45%, 50%, 60%, 65%, 70%, 75%, 80%, 85%, 90%, or 95% of subjects treated according to the method and/or with the provided articles of manufacture or compositions do not exhibit early onset CRS or neurotoxicity and/or do not exhibit onset of CRS earlier than 1 day, 2 days, 3 days or 4 days following initiation of the administration. In some embodiments, at least at or about 45%, 50%, 60%, 65%, 70%, 75%, 80%, 85%, 90%, or 95% of subjects treated according to the methods, and/or with the provided articles of manufacture or compositions, do not exhibit onset of neurotoxicity earlier than 3 days, 4 days, 5 days, six days or 7 days following initiation of the administration. In some aspects, the median onset of neurotoxicity among subjects treated according to the methods, and/or with the provided articles of manufacture or compositions, is at or after the median peak of, or median time to resolution of, CRS in subjects treated according to the method. In some cases, the median onset of neurotoxicity among subjects treated according to the method is greater than at or about 8, 9, 10, or 11 days.

### II. CELL THERAPY AND ENGINEERING CELLS

In some embodiments, the cell therapy (e.g., T cell therapy) for use in accord with the provided combination therapy methods includes administering engineered cells expressing recombinant receptors (e.g. CAR) designed to recognize and/or specifically bind to an antigen associated with the disease or condition, such as r/r/FL Grade 1-3A or r/r/MZL, including such diseases or conditions exhibiting high-risk features. In particular embodiments, the antigen that is bound or recognized by the recombinant receptor (e.g. CAR) is CD19. In some embodiments, binding to the antigen results in a response, such as an immune response against such antigen. In some embodiments, the cells contain or are engineered to contain the recombinant receptor, such as a chimeric antigen receptor (CAR). The recombinant receptor, such as a CAR, generally includes an extracellular antigen (or ligand) binding domain specific to the antigen that is linked to one or more intracellular signaling components, in some aspects via linkers and/or transmembrane domain(s). In some aspects, the engineered cells are provided as pharmaceutical compositions and formulations suitable for administration to a subjects, such as for adoptive cell therapy. Also provided are therapeutic methods for administering the cells and compositions to subjects, e.g., patients.

In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

A. Chimeric Antigen Receptors

In some embodiments of the provided methods and uses, the engineered cells, such as T cells, express a chimeric receptors, such as a chimeric antigen receptors (CAR), that contains one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. Upon specific binding to the molecule, e.g., antigen, the receptor generally delivers an immunostimulatory signal, such as an ITAM-transduced signal, into the cell, thereby promoting an immune response targeted to the disease or condition. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No. WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

In some embodiments, the engineered cells, such as T cells, express a recombinant receptor such as a chimeric antigen receptor (CAR) with specificity for a particular antigen (or marker or ligand), such as an antigen expressed on the surface of a particular cell type. In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30. In particular aspects, the antigen is CD19. In some embodiments, any of such antigens are antigens expressed on human B cells.

The chimeric receptors, such as CARs, generally include an extracellular antigen binding domain that is an antigen-binding portion or portions of an antibody molecule. In some embodiments, the antigen-binding domain is a portion of an antibody molecule, generally a variable heavy (V_{H}) chain region and/or variable light (V_{L}) chain region of the antibody, e.g., an scFv antibody fragment. In some embodiments, the antigen-binding domain is a single domain antibody (sdAb), such as sdFv, nanobody, V_{H}H and V_{NAR}. In some embodiments, an antigen-binding fragment comprises antibody variable regions joined by a flexible linker.

In some embodiments, the antibody or an antigen-binding fragment (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19. In some embodiments, the antigen is CD19. In some embodiments, the scFv contains a V_{H} and a V_{L} derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments the antigen-binding domain includes a V_{H} and/or V_{L} derived from FMC63, which, in some aspects, can be an scFv. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises CDR-H1 and CDR-H2 set forth in SEQ ID NO: 38 and 39, respectively, and CDR-H3 set forth in SEQ ID NO: 40 or 54 and CDR-L1 set forth in SEQ ID NO: 35 and CDR-L2 set forth in SEQ ID NO: 36 or 55 and CDR-L3 sequences set forth in SEQ ID NO: 37 or 56. In some embodiments, the FMC63 antibody comprises the heavy chain variable region (V_{H}) comprising the amino acid sequence of SEQ ID NO: 41 and the light chain variable region (V_{L}) comprising the amino acid sequence of SEQ ID NO: 42.

In some embodiments, the scFv comprises a variable light chain containing the CDR-L1 sequence of SEQ ID NO:35, a CDR-L2 sequence of SEQ ID NO:36, and a CDR-L3 sequence of SEQ ID NO:37 and/or a variable heavy chain containing a CDR-H1 sequence of SEQ ID NO:38, a CDR-H2 sequence of SEQ ID NO:39, and a CDR-H3 sequence of SEQ ID NO:40, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of FMC63 set forth in SEQ ID NO:41 and a variable light chain region of FMC63 set forth in SEQ ID NO:42, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:59. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:57 or a sequence that exhibits at least 85%, 86%, 87%, 88%, 89%, 90%, 91%, 92%, 93%, 94%, 95%, 96%, 97%, 98%, or 99% sequence identity to SEQ ID NO:57. In some embodiments, the scFv comprises the sequence of amino acids set forth in SEQ ID NO:43 or a sequence that exhibits at least 85%, 86%, 87%, 88%, 89%, 90%, 91%, 92%, 93%, 94%, 95%, 96%, 97%, 98%, or 99% sequence identity to SEQ ID NO:43.

In some embodiments the antigen-binding domain includes a V_{H} and/or V_{L} derived from SJ25C1, which, in some aspects, can be an scFv. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDR-H1, CDR-H2 and CDR-H3 set forth in SEQ ID NOS: 47-49, respectively, and CDR-L1, CDR-L2 and CDR-L3 sequences set forth in SEQ ID NOS: 44-46, respectively. In some embodiments, the SJ25C1 antibody comprises the heavy chain variable region (V_{H}) comprising the amino acid sequence of SEQ ID NO: 50 and the light chain variable region (V_{L}) comprising the amino acid sequence of SEQ ID NO: 51. In some embodiments, the svFv comprises a variable light chain containing a CDR-L1 sequence of SEQ ID NO:44, a CDR-L2 sequence of SEQ ID NO: 45, and a CDR-L3 sequence of SEQ ID NO:46 and/or a variable heavy chain containing a CDR-H1 sequence of SEQ ID NO:47, a CDR-H2 sequence of SEQ ID NO:48, and a CDR-H3 sequence of SEQ ID NO:49, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of SJ25C1 set forth in SEQ ID NO:50 and a variable light chain region of SJ25C1 set forth in SEQ ID NO:51, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:52. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:53 or a sequence that exhibits at least 85%, 86%, 87%, 88%, 89%, 90%, 91%, 92%, 93%, 94%, 95%, 96%, 97%, 98%, or 99% sequence identity to SEQ ID NO:53.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody, V_{H}H or V_{NAR}) or fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. In some aspects, the CAR is a bispecific CAR, e.g., containing two antigen-binding domains with different specificities.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

The terms “complementarity determining region,” and “CDR,” synonymous with “hypervariable region” or “HVR,” are known, in some cases, to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known, in some cases, to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 5, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 5
Boundaries of CDRs according to various numbering schemes|

| CDR | Kabat | Chothia | AbM | Contact |
|---|---|---|---|---|
| CDR-L1 | L24-L34 | L24-L34 | L24-L34 | L30-L36 |
| CDR-L2 | L50-L56 | L50-L56 | L50-L56 | L46-L55 |
| CDR-L3 | L89-L97 | L89-L97 | L89-L97 | L89-L96 |
| CDR-H1 | H31-H35B | H26-H32 . . . 34 | H26--H35B | H30-H35B |
| (Kabat | | | | |
| Numbering¹) | | | | |
| CDR-H1 | H31-H35 | H26-H32 | H26-H35 | H30-H35 |
| (Chothia | | | | |
| Numbering²) | | | | |
| CDR-H2 | H50-H65 | H52-H56 | H50-H58 | H47-H58 |
| CDR-H3 | H95-H102 | H95-H102 | H95-H102 | H93-H101 |

¹Kabat et al. (1991), “Sequences of Proteins of Immunological Interest,’’ 5th Ed. Public Health Service, National Institutes of Health, Bethesda, MD
²Al-Lazikani et al., (1997) JMB 273,927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W. H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W. H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies (sdAb) are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known. Exemplary single-domain antibodies include sdFv, nanobody, V_{H}H or V_{NAR}.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In some aspects, the recombinant receptor, e.g., a chimeric antigen receptor, includes an extracellular portion containing one or more ligand- (e.g., antigen-) binding domains, such as an antibody or fragment thereof, and one or more intracellular signaling region or domain (also interchangeably called a cytoplasmic signaling domain or region). In some aspects, the recombinant receptor, e.g., CAR, further includes a spacer and/or a transmembrane domain or portion. In some aspects, the spacer and/or transmembrane domain can link the extracellular portion containing the ligand- (e.g., antigen-) binding domain and the intracellular signaling region(s) or domain(s)

In some embodiments, the recombinant receptor such as the CAR, further includes a spacer, which may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, Hudecek et al. (2015) Cancer Immunol Res. 3(2): 125-135 or international patent application publication number WO2014031687.

In some embodiments, the spacer contains only a hinge region of an IgG, such as only a hinge of IgG4 or IgG1, such as the hinge only spacer set forth in SEQ ID NO: 1, and encoded by the sequence set forth in SEQ ID NO: 2. In some embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO: 3. In some embodiments, the spacer the spacer is an Ig hinge, e.g., an IgG4 hinge, linked to a C_{H}3 domain only, such as set forth in SEQ ID NO: 4. In some embodiments, the spacer is or comprises a glycine-serine rich sequence or other flexible linker such as known flexible linkers. In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5.

In some aspects, the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) consists or comprises the sequence of amino acids set forth in SEQ ID NOS: 1, 3-5, 27-34 or 58, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 (4-1BB), or CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28 or a variant thereof. The extracellular domain and transmembrane can be linked directly or indirectly. In some embodiments, the extracellular domain and transmembrane are linked by a spacer, such as any described herein.

In some embodiments, the transmembrane domain of the receptor, e.g., the CAR is a transmembrane domain of human CD28 or variant thereof, e.g., a 27-amino acid transmembrane domain of a human CD28 (Accession No. P10747.1), or is a transmembrane domain that comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:8. In some embodiments, the transmembrane-domain containing portion of the recombinant receptor comprises the sequence of amino acids set forth in SEQ ID NO: 9 or a sequence of amino acids having at least or at least about 85%, 86%, 87%, 88%, 89%, 90%, 91%, 92%, 93%, 94%, 95%, 96%, 97%, 98%, 99% or more sequence identity thereto.

In some embodiments, the recombinant receptor, e.g., CAR, includes at least one intracellular signaling component or components, such as an intracellular signaling region or domain. T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components. Among the intracellular signaling region are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

In some embodiments, upon ligation of the CAR, the cytoplasmic domain or intracellular signaling region of the CAR activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling region of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability. In some embodiments, the intracellular signaling regions, e.g., comprising intracellular domain or domains, include the cytoplasmic sequences of a region or domain that is involved in providing costimulatory signal.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8alpha, CD8beta, CD4, CD25, or CD16. For example, in some aspects, the CAR or other chimeric receptor includes a chimeric molecule between CD3-zeta (CD3-ζ) or Fc receptor γ and CD8alpha, CD8beta, CD4, CD25 or CD16.

In some embodiments, the intracellular (or cytoplasmic) signaling region comprises a human CD3 chain, optionally a CD3 zeta stimulatory signaling domain or functional variant thereof, such as an 112 AA cytoplasmic domain of isoform 3 of human CD3ζ (Accession No. P20963.2) or a CD3 zeta signaling domain as described in U.S. Pat. No. 7,446,190 or U.S. Pat. No. 8,911,993. In some embodiments, the intracellular signaling region comprises the sequence of amino acids set forth in SEQ ID NO: 13, 14 or 15 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13, 14 or 15.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40 (CD134), CD27, DAP10, DAP12, ICOS and/or other costimulatory receptors. In some embodiments, the CAR includes a costimulatory region or domain of CD28 or 4-1BB, such as of human CD28 or human 4-1BB.

In some embodiments, the intracellular signaling region or domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 10 or 11 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular region comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 12 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 12.

In some aspects, the same CAR includes both the primary (or activating) cytoplasmic signaling regions and costimulatory signaling components.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g. iCAR) and includes intracellular components that dampen or suppress an immune response, such as an ITAM-and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

An exemplary polypeptide for a truncated EGFR (e.g. tEGFR) comprises the sequence of amino acids set forth in SEQ ID NO: 7 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16. An exemplary T2A linker sequence comprises the sequence of amino acids set forth in SEQ ID NO: 6 or 17 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 17.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, the chimeric antigen receptor includes an extracellular portion containing the antibody or fragment described herein. In some aspects, the chimeric antigen receptor includes an extracellular portion containing the antibody or fragment described herein and an intracellular signaling domain. In some embodiments, the antibody or fragment includes an scFv or a single-domain V_{H} antibody and the intracellular domain contains an ITAM. In some aspects, the intracellular signaling domain includes a signaling domain of a zeta chain of a CD3-zeta (CD3ζ) chain. In some embodiments, the CD3-zeta chain is a human CD3-zeta chain. In some embodiments, the intracellular signaling region further comprises a CD28 and CD137 (4-1BB, TNFRSF9) co-stimulatory domains, linked to a CD3 zeta intracellular domain. In some embodiments, the CD28 is a human CD28. In some embodiments, the 4-1BB is a human 4-1BB. In some embodiments, the chimeric antigen receptor includes a transmembrane domain disposed between the extracellular domain and the intracellular signaling region. In some aspects, the transmembrane domain contains a transmembrane portion of CD28. The extracellular domain and transmembrane can be linked directly or indirectly. In some embodiments, the extracellular domain and transmembrane are linked by a spacer, such as any described herein.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. For example, in some embodiments, the CAR includes an antibody such as an antibody fragment, including scFvs, e.g. specific for CD19 such as any described above, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain.

In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer. In some embodiments, the CAR includes an antibody or fragment, such as scFv, e.g. specific for CD19 such as any described above, a spacer such as any of the Ig-hinge containing spacers, a CD28-derived transmembrane domain, a 4-1BB-derived intracellular signaling domain, and a CD3 zeta-derived signaling domain.

In particular embodiments of any of the provided methods, the CAR contains in order from N-terminus to C-terminus an extracellular antigen-binding domain that is the scFv set forth in SEQ ID NO: 43, the spacer set forth in SEQ ID NO:1, the transmembrane domain set forth in SEQ ID NO:8, the 4-1BB costimulatory signaling domain set forth in SEQ ID NO:12, and the signaling domain of a CD3-zeta (CD3ζ) chain set forth in SEQ ID NO:13.

B. Nucleic Acids, Vectors and Methods for Genetic Engineering

In some embodiments, the cells, e.g., T cells, are genetically engineered to express a recombinant receptor. In some embodiments, the engineering is carried out by introducing polynucleotides that encode the recombinant receptor. Also provided are polynucleotides encoding a recombinant receptor, and vectors or constructs containing such nucleic acids and/or polynucleotides.

In some cases, the nucleic acid sequence encoding the recombinant receptor contains a signal sequence that encodes a signal peptide. In some aspects, the signal sequence may encode a signal peptide derived from a native polypeptide. In other aspects, the signal sequence may encode a heterologous or non-native signal peptide, such as the exemplary signal peptide of the GMCSFR alpha chain set forth in SEQ ID NO:25 and encoded by the nucleotide sequence set forth in SEQ ID NO:24. In some cases, the nucleic acid sequence encoding the recombinant receptor, e.g., chimeric antigen receptor (CAR) contains a signal sequence that encodes a signal peptide. Non-limiting exemplary examples of signal peptides include, for example, the GMCSFR alpha chain signal peptide set forth in SEQ ID NO: 25 and encoded by the nucleotide sequence set forth in SEQ ID NO:24, or the CD8 alpha signal peptide set forth in SEQ ID NO:26.

In some embodiments, the polynucleotide encoding the recombinant receptor contains at least one promoter that is operatively linked to control expression of the recombinant receptor. In some examples, the polynucleotide contains two, three, or more promoters operatively linked to control expression of the recombinant receptor.

In certain cases where nucleic acid molecules encode two or more different polypeptide chains, e.g., a recombinant receptor and a marker, each of the polypeptide chains can be encoded by a separate nucleic acid molecule. For example, two separate nucleic acids are provided, and each can be individually transferred or introduced into the cell for expression in the cell. In some embodiments, the nucleic acid encoding the recombinant receptor and the nucleic acid encoding the marker are operably linked to the same promoter and are optionally separated by an internal ribosome entry site (IRES), or a nucleic acid encoding a self-cleaving peptide or a peptide that causes ribosome skipping, which optionally is a T2A, a P2A, an E2A or an F2A. In some embodiments, the nucleic acids encoding the marker and the nucleic acid encoding the recombinant receptor are operably linked to two different promoters. In some embodiments, the nucleic acid encoding the marker and the nucleic acid encoding the recombinant receptor are present or inserted at different locations within the genome of the cell. In some embodiments, the polynucleotide encoding the recombinant receptor is introduced into a composition containing cultured cells, such as by retroviral transduction, transfection, or transformation.

In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products ((e.g. encoding the marker and encoding the recombinant receptor) by a message from a single promoter. Alternatively, in some cases, a single promoter may direct expression of an RNA that contains, in a single open reading frame (ORF), two or three genes (e.g. encoding the marker and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004)). Various 2A elements are known. Examples of 2A sequences that can be used in the methods and system disclosed herein, without limitation, 2A sequences from the foot-and-mouth disease virus (F2A, e.g., SEQ ID NO: 21), equine rhinitis A virus (E2A, e.g., SEQ ID NO: 20), Thosea asigna virus (T2A, e.g., SEQ ID NO: 6 or 17), and porcine teschovirus-1 (P2A, e.g., SEQ ID NO: 18 or 19) as described in U.S. Patent Publication No. 20070116690.

Any of the recombinant receptors described herein can be encoded by polynucleotides containing one or more nucleic acid sequences encoding recombinant receptors, in any combinations or arrangements. For example, one, two, three or more polynucleotides can encode one, two, three or more different polypeptides, e.g., recombinant receptors. In some embodiments, one vector or construct contains a nucleic acid sequence encoding marker, and a separate vector or construct contains a nucleic acid sequence encoding a recombinant receptor, e.g., CAR. In some embodiments, the nucleic acid encoding the marker and the nucleic acid encoding the recombinant receptor are operably linked to two different promoters. In some embodiments, the nucleic acid encoding the recombinant receptor is present downstream of the nucleic acid encoding the marker.

In some embodiments, the vector backbone contains a nucleic acid sequence encoding one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:7 or 16) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR).

In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 7 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16.

In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy, 2014 Apr. 3. doi: 10.1038/gt.2014.25; Carlens et al. (2000) Exp. Hematol., 28(10): 1137-46; Alonso-Camino et al. (2013) Mol. Ther. Nucl. Acids., 2, e93; Park et al., Trends Biotechnol., 2011 Nov. 29(11): 550-557.

In some embodiments, the viral vector is an adeno-associated virus (AAV).

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV) or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No. WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the anti-CD3/anti-CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

Cells and Preparation of Cells for Genetic Engineering In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood, 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynabeads® or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody-or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS®) (Miltenyi Biotec, Auburn, Calif.). Magnetic Activated Cell Sorting (MACS®) systems are capable of high-purity selection of cells having magnetized particles attached thereto. In certain embodiments, MACS® operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labeled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS® system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS® system in some aspects uses antibody-coupled magnetizable particles that are supplied in a sterile, non-pyrogenic solution. In some embodiments, after labeling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labeled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy® system (Miltenyi Biotec). The CliniMACS Prodigy® system in some aspects is equipped with a cell processing unity that permits automated washing and fractionation of cells by centrifugation. The CliniMACS Prodigy® system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy® system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (fluorescence activated cell sorting, FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° C. per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating or stimulating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/mL). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees Celsius, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

C. Methods of Engineering Cells

In particular embodiments, the engineered cells are produced by a process that generates an output composition of enriched T cells from one or more input compositions and/or from a single biological sample. In certain embodiments, the output composition contains cells that express a recombinant receptor, e.g., a CAR, such as an anti-CD19 CAR. In particular embodiments, the cells of the output compositions are suitable for administration to a subject as a therapy, e.g., an autologous cell therapy. In some embodiments, the output composition is a composition of enriched CD4+ or CD8+ T cells.

In some embodiments, the process for generating or producing engineered cells is by a process that includes some or all of the steps of: collecting or obtaining a biological sample; isolating, selecting, or enriching input cells from the biological sample; cryopreserving and storing the input cells; thawing and/or incubating the input cells under stimulating conditions; engineering the stimulated cells to express or contain a recombinant polynucleotide, e.g., a polynucleotide encoding a recombinant receptor such as a CAR; cultivating the engineered cells, e.g. to a threshold amount, density, or expansion; formulating the cultivated cells in an output composition; and/or cryopreserving and storing the formulated output cells until the cells are released for infusion and/or are suitable to be administered to a subject. In certain embodiments, the process is performed with two or more input compositions of enriched T cells, such as a separate CD4+ composition and a separate CD8+ composition, that are separately processed and engineered from the same starting or initial biological sample and re-infused back into the subject at a defined ratio, e.g. 1:1 ratio of CD4+ to CD8+ T cells. In some embodiments, the enriched T cells are or include engineered T cells, e.g., T cells transduced to express a recombinant receptor.

In particular embodiments, an output composition of engineered cells expressing a recombinant receptor (e.g. anti-CD19 CAR) is produced from an initial and/or input composition of cells. In some embodiments, the input composition is a composition of enriched T cells, enriched CD4+ T cells, and/or enriched CD8+ T cells (herein after also referred to as compositions of enriched T cells, compositions of enriched CD4+ T cells, and compositions of enriched CD8+ T cells, respectively). In some embodiments, a composition enriched in CD4+ T cells contains at least 60%, 65%, 70%, 75%, 80%, 85%, 90%, 95%, 98%, 99%, or 99.9% CD4+ T cells. In particular embodiments, the composition of enriched CD4+ T cells contains 100% CD4+ T cells contains about 100% CD4+ T cells. In certain embodiments, the composition of enriched T cells includes or contains less than 20%, less than 10%, less than 5%, less than 1%, less than 0.1%, or less than 0.01% CD8+ T cells, and/or contains no CD8+ T cells, and/or is free or substantially free of CD8+ T cells. In some embodiments, the populations of cells consist essentially of CD4+ T cells. In some embodiments, a composition enriched in CD8+ T cells contains at least 75%, 80%, 85%, 90%, 95%, 98%, 99%, or 99.9% CD8+ T cells, or contains or contains about 100% CD8+ T cells. In certain embodiments, the composition of enriched CD8+ T cells includes or contains less than 20%, less than 10%, less than 5%, less than 1%, less than 0.1%, or less than 0.01% CD4+ T cells, and/or contains no CD4+ T cells, and/or is free or substantially free of CD4+ T cells. In some embodiments, the populations of cells consist essentially of CD8+ T cells.

In certain embodiments, the process for producing engineered cells further can include one or more of: activating and/or stimulating a cells, e.g., cells of an input composition; genetically engineering the activated and/or stimulated cells, e.g., to introduce a polynucleotide encoding a recombinant protein by transduction or transfection; and/or cultivating the engineered cells, e.g., under conditions that promote proliferation and/or expansion. In particular embodiments, the provided methods may be used in connection with harvesting, collecting, and/or formulating output compositions produced after the cells have been incubated, activated, stimulated, engineered, transduced, transfected, and/or cultivated.

In some embodiments, engineered cells, such as those that express an anti-CD19 CAR as described, used in accord with the provided methods and uses are produced or generated by a process for selecting, isolating, activating, stimulating, expanding, cultivating, and/or formulating cells. In some embodiments, such methods include any as described.

In some embodiments, engineered cells, such as those that express an anti-CD19 CAR as described, used in accord with the provided methods and uses are produced or generated by exemplary processes as described in, for example, WO 2019/089855 and WO 2015/164675.

In some of any embodiments, exemplary processes for generating, producing or manufacturing the engineered cells, such as those that express an anti-CD19 CAR as described, or a composition comprising such cells, such as a composition comprising engineered CD4+ T cells and engineered CD8+ T cells each expressing the same anti-CD19 chimeric antigen receptor (CAR), involve subjecting enriched CD4+ and enriched CD8+ cell populations, separately, to process steps. In some aspects of the exemplary process for generating or manufacturing engineered cells, CD4+ and CD8+ cells are separately selected from human peripheral blood mononuclear cells (PBMCs), for example, that are obtained by leukapheresis, generating separate enriched CD4+ and enriched CD8+ cell compositions. In some aspects, such cells can be cryopreserved. In some aspects, the CD4+ and CD8+ compositions can be subsequently thawed and separately subject to steps for stimulation, transduction, and expansion.

In some aspects of the exemplary process for generating or manufacturing engineered cells, thawed CD4+ and CD8+ cells are separately stimulated, for example, in the presence of paramagnetic polystyrene-coated beads coupled to anti-CD3 and anti-CD28 antibodies (such as at a 1:1 bead to cell ratio). In some aspects, the stimulation is carried out in media containing human recombinant IL-2, human recombinant IL-15, and N-Acetyl Cysteine (NAC). In some aspects, the cell culture media for CD4+ cells also can include human recombinant IL-7.

In some aspects of the exemplary process for generating or manufacturing engineered cells, following the introduction of the beads, CD4+ and CD8+ cells are separately transduced with a lentiviral vector encoding the same CAR, such as the same anti-CD19 CAR. In some aspects, the CAR can contain an anti-CD19 scFv derived from a murine antibody, an immunoglobulin spacer, a transmembrane domain derived from CD28, a costimulatory region derived from 4-1BB, and a CD3-zeta intracellular signaling domain. In some aspects, the vector can encode a truncated receptor that serves as a surrogate marker for CAR expression that is connected to the CAR construct by a T2A sequence. In some aspects of the exemplary process, the cells are transduced in the presence of 10 μg/ml protamine sulfate.

In some aspects of the exemplary process for generating or manufacturing engineered cells, following transduction, the beads are removed from the cell compositions by exposure to a magnetic field. In some aspects, the CD4+ and CD8+ cell compositions are separately cultivated for expansion with continual mixing and oxygen transfer by a bioreactor (for example, a Xuri™ W25 Bioreactor). In some cases, poloxamer is added to the media. In some aspects, both the CD4+ and the CD8+ cell compositions are cultivated in the presence of IL-2 and IL-15. In some aspects, the CD4+ cell media also includes IL-7. In some cases, the CD4+ and CD8+ cells are each cultivated, prior to harvest, to 4-fold expansion. In some aspects, one day after reaching the threshold, cells from each composition can be separately harvested, formulated, and cryopreserved. In some aspects, the exemplary processes for generating, producing or manufacturing the engineered cells, such as those that express an anti-CD19 CAR as described, or a composition comprising such cells, such as a composition comprising engineered CD4+ T cells and engineered CD8+ T cells each expressing the same anti-CD19 chimeric antigen receptor (CAR), include those described in Table 6 below.

TABLE 6
Exemplary process for generating CD4+ and CD8+ CAR-T cells

| Stage | CD4+ cells | CD8+ cells |
|---|---|---|
| Stimulation | anti-CD3/CD28 antibody | anti-CD3/CD28 antibody |
| (day 1-2) | conjugated beads | conjugated beads |
| | 1:1 bead to cell ratio | 1:1 bead to cell ratio |
| | media: IL-2, IL-7, IL-15, and NAC | media: IL-2, IL-15, and NAC |
| Transduction | transduction adjuvant (e.g. 10 | transduction adjuvant (e.g. 10 |
| (day 2-5) | μg/ml protamine sulfate) | μg/ml protamine sulfate) |
| Bead removal | magnetic bead removal | magnetic bead removal |
| (day 5*) | | |
| Expansion | rocking motion bioreactor and/or | rocking motion bioreactor and/or |
| (day 5*-Harvest) | continuous mixing | continuous mixing |
| | media: IL-2, IL-7, IL15, and | media: IL-2, IL15, and poloxamer |
| | poloxamer | |

*Approximate

In other aspects, a different exemplary process for generating, producing or manufacturing the engineered cells or a composition comprising such cells include a process that differs from the exemplary process above in that: NAC is not added to the media during stimulation; CD4+ cell media does not contain IL-2; cells are stimulated at a bead to cell ratio of 3:1; cells are transduced with a higher concentration of protamine sulfate; bead removal occurs at about day 7; and expansion is performed at a static setting, i.e., without continual mixing or perfusion (e.g., semi-continuous and/or stepwise perfusion), and without poloxamer.

In some embodiments, at least one separate composition of enriched CD4+ T cells and at least one separate composition of enriched CD8+ T cells are isolated, selected, enriched, or obtained from a single biological sample, e.g., a sample of PBMCs or other white blood cells from the same donor such as a patient or healthy individual. In some embodiments, a separate composition of enriched CD4+ T cells and a separate composition of enriched CD8+ T cells originated, e.g., were initially isolated, selected, and/or enriched, from the same biological sample, such as a single biological sample obtained, collected, and/or taken from a single subject. In some embodiments, a biological sample is first subjected to selection of CD4+ T cells, where both the negative and positive fractions are retained, and the negative fraction is further subjected to selection of CD8+ T cells. In other embodiments, a biological sample is first subjected to selection of CD8+ T cells, where both the negative and positive fractions are retained, and the negative fraction is further subjected to selection of CD4+ T cells. In some embodiments, methods of selection are carried out as described in International PCT publication No. WO2015/164675. In some embodiments, methods of selection are carried out as described in International PCT publication No. WO 2019/089855. In some aspects, a biological sample is first positively selected for CD8+ T cells to generate at least one composition of enriched CD8+ T cells, and the negative fraction is then positively selected for CD4+ T cells to generate at least one composition of enriched CD4+ T cells, such that the at least one composition of enriched CD8+ T cells and the at least one composition of enriched CD4+ T cells are separate compositions from the same biological sample, e.g., from the same donor patient or healthy individual. In some aspects, two or more separate compositions of enriched T cells, e.g., at least one being a composition of enriched CD4+ T cells and at least one being a separate composition of enriched CD8+ T cells from the same donor, are separately frozen, e.g., cryoprotected or cryopreserved in a cryopreservation media.

In some aspects, two or more separate compositions of enriched T cells, e.g., at least one being a composition of enriched CD4+ T cells and at least one being a separate composition of enriched CD8+ T cells from the same biological sample, are activated and/or stimulated by contacting with a stimulatory reagent (e.g., by incubation with CD3/CD28 conjugated magnetic beads for T cell activation). In some aspects, each of the activated/stimulated cell composition is engineered, transduced, and/or transfected, e.g., using a viral vector encoding a recombinant protein (e.g. CAR), to express the same recombinant protein in the CD4+ T cells and CD8+ T cells of each cell composition. In some aspects, the method comprises removing the stimulatory reagent, e.g., magnetic beads, from the cell composition. In some aspects, a cell composition containing engineered CD4+ T cells and a cell composition containing engineered CD8+ T cells are separately cultivated, e.g., for separate expansion of the CD4+ T cell and CD8+ T cell populations therein. In certain embodiments, a cell composition from the cultivation is harvested and/or collected and/or formulated, e.g., by washing the cell composition in a formulation buffer. In certain embodiments, a formulated cell composition comprising CD4+ T cells and a formulated cell composition comprising CD8+ T cells is frozen, e.g., cryoprotected or cryopreserved in a cryopreservation media. In some aspects, engineered CD4+ T cells and CD8+ T cells in each formulation originate from the same donor or biological sample and express the same recombination protein (e.g., CAR, such as anti-CD19 CAR). In some aspects, a separate engineered CD4+ formulation and a separate engineered CD8+ formulation are administered at a defined ratio, e.g. 1:1, to a subject in need thereof such as the same donor.

1. Cells and Preparation of Cells for Genetic Engineering

In some embodiments, cells, such as T cells, used in connection with the provided methods, uses, articles of manufacture or compositions are cells have been genetically engineered to express a recombinant receptor, e.g., a CAR or a TCR described herein. In some embodiments, the engineered cells are used in the context of cell therapy, e.g., adoptive cell therapy. In some embodiments, the engineered cells are immune cells. In some embodiments, the engineered cells are T cells, such as CD4+ or CD8+ T cells. In some embodiments, the engineered cells are T cells, such as CD4+ and CD8+ T cells.

In some embodiments, the nucleic acids, such as nucleic acids encoding a recombinant receptor, are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, the cells are autologous. In some aspects, the cells are allogeneic. Among the methods included are off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4⁺ and/or of CD8⁺ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll® or Ficoll® gradient.

In some embodiments, at least a portion of the selection step includes incubation of cells with a selection reagent. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In some embodiments, any known method using a selection reagent or reagents for separation based on such markers may be used. In some embodiments, the selection reagent or reagents result in a separation that is affinity- or immunoaffinity-based separation. For example, the selection in some aspects includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

In some aspects of such processes, a volume of cells is mixed with an amount of a desired affinity-based selection reagent. The immunoaffinity-based selection can be carried out using any system or method that results in a favorable energetic interaction between the cells being separated and the molecule specifically binding to the marker on the cell, e.g., the antibody or other binding partner on the solid surface, e.g., particle. In some embodiments, methods are carried out using particles such as beads, e.g. magnetic beads, that are coated with a selection agent (e.g. antibody) specific to the marker of the cells. The particles (e.g. beads) can be incubated or mixed with cells in a container, such as a tube or bag, while shaking or mixing, with a constant cell density-to-particle (e.g., bead) ratio to aid in promoting energetically favored interactions. In other cases, the methods include selection of cells in which all or a portion of the selection is carried out in the internal cavity of a centrifugal chamber, for example, under centrifugal rotation. In some embodiments, incubation of cells with selection reagents, such as immunoaffinity-based selection reagents, is performed in a centrifugal chamber. In certain embodiments, the isolation or separation is carried out using a system, device, or apparatus described in International Patent Application, Publication Number WO2009/072003, or US 20110003380 A1. In one example, the system is a system as described in International Publication Number WO2016/073602.

In some embodiments, by conducting such selection steps or portions thereof (e.g., incubation with antibody-coated particles, e.g., magnetic beads) in the cavity of a centrifugal chamber, the user is able to control certain parameters, such as volume of various solutions, addition of solution during processing and timing thereof, which can provide advantages compared to other available methods. For example, the ability to decrease the liquid volume in the cavity during the incubation can increase the concentration of the particles (e.g. bead reagent) used in the selection, and thus the chemical potential of the solution, without affecting the total number of cells in the cavity. This in turn can enhance the pairwise interactions between the cells being processed and the particles used for selection. In some embodiments, carrying out the incubation step in the chamber, e.g., when associated with the systems, circuitry, and control as described herein, permits the user to effect agitation of the solution at desired time(s) during the incubation, which also can improve the interaction.

In some embodiments, at least a portion of the selection step is performed in a centrifugal chamber, which includes incubation of cells with a selection reagent. In some aspects of such processes, a volume of cells is mixed with an amount of a desired affinity-based selection reagent that is far less than is normally employed when performing similar selections in a tube or container for selection of the same number of cells and/or volume of cells according to manufacturer's instructions. In some embodiments, an amount of selection reagent or reagents that is/are no more than 5%, no more than 10%, no more than 15%, no more than 20%, no more than 25%, no more than 50%, no more than 60%, no more than 70% or no more than 80% of the amount of the same selection reagent(s) employed for selection of cells in a tube or container-based incubation for the same number of cells and/or the same volume of cells according to manufacturer's instructions is employed.

In some embodiments, for selection, e.g., immunoaffinity-based selection of the cells, the cells are incubated in the cavity of the chamber in a composition that also contains the selection buffer with a selection reagent, such as a molecule that specifically binds to a surface marker on a cell that it desired to enrich and/or deplete, but not on other cells in the composition, such as an antibody, which optionally is coupled to a scaffold such as a polymer or surface, e.g., bead, e.g., magnetic bead, such as magnetic beads coupled to monoclonal antibodies specific for CD4 and CD8. In some embodiments, as described, the selection reagent is added to cells in the cavity of the chamber in an amount that is substantially less than (e.g. is no more than 5%, 10%, 20%, 30%, 40%, 50%, 60%, 70% or 80% of the amount) as compared to the amount of the selection reagent that is typically used or would be necessary to achieve about the same or similar efficiency of selection of the same number of cells or the same volume of cells when selection is performed in a tube with shaking or rotation. In some embodiments, the incubation is performed with the addition of a selection buffer to the cells and selection reagent to achieve a target volume with incubation of the reagent of, for example, 10 mL to 200 mL, such as at least or at least about 10 mL, 20 mL, 30 mL, 40 mL, 50 mL, 60 mL, 70 mL, 80 mL, 90 mL, 100 mL, 150 mL or 200 mL. In some embodiments, the selection buffer and selection reagent are pre-mixed before addition to the cells. In some embodiments, the selection buffer and selection reagent are separately added to the cells. In some embodiments, the selection incubation is carried out with periodic gentle mixing condition, which can aid in promoting energetically favored interactions and thereby permit the use of less overall selection reagent while achieving a high selection efficiency.

In some embodiments, the total duration of the incubation with the selection reagent is from or from about 5 minutes to 6 hours, such as from or from about 30 minutes to 3 hours, for example, at least or at least about 30 minutes, 60 minutes, 120 minutes or 180 minutes.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, such process is carried out within the entirely closed system to which the chamber is integral. In some embodiments, this process (and in some aspects also one or more additional step, such as a previous wash step washing a sample containing the cells, such as an apheresis sample) is carried out in an automated fashion, such that the cells, reagent, and other components are drawn into and pushed out of the chamber at appropriate times and centrifugation effected, so as to complete the wash and binding step in a single closed system using an automated program.

In some embodiments, after the incubation and/or mixing of the cells and selection reagent and/or reagents, the incubated cells are subjected to a separation to select for cells based on the presence or absence of the particular reagent or reagents. In some embodiments, the separation is performed in the same closed system in which the incubation of cells with the selection reagent was performed. In some embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In particular embodiments, a biological sample, e.g., a sample of PBMCs or other white blood cells, are subjected to selection of CD4+ T cells, where both the negative and positive fractions are retained. In certain embodiments, CD8+ T cells are selected from the negative fraction. In some embodiments, a biological sample is subjected to selection of CD8+ T cells, where both the negative and positive fractions are retained. In certain embodiments, CD4+ T cells are selected from the negative fraction.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In some embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynabeads® or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084, are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody-or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS®) (Miltenyi Biotec, Auburn, Calif.). Magnetic Activated Cell Sorting (MACS®) systems are capable of high-purity selection of cells having magnetized particles attached thereto. In certain embodiments, MACS® operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labeled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS® system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS® system in some aspects uses antibody-coupled magnetizable particles that are supplied in a sterile, non-pyrogenic solution. In some embodiments, after labeling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labeled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy® system (Miltenyi Biotec). The CliniMACS Prodigy® system in some aspects is equipped with a cell processing unity that permits automated washing and fractionation of cells by centrifugation. The CliniMACS Prodigy® system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy® system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. In some embodiments, the cell compositions are stored in a formulation containing at or about 5%, 6%, 7%, 7.5%, 8%, 9% or 10% dimethylsulfoxide, or a range defined by any of the foregoing, such as at or about 7.5% DMSO. In some aspects, the compositions are stored in a formulation containing at or about 0.5%, 1%, 2% or 2.5% (v/v) of 25% human albumin, or a range defined by any of the foregoing, such as at or about 1% (v/v) 25% human albumin The cells are generally then frozen to −80° C. at a rate of 1° per minute and stored in the vapor phase of a liquid nitrogen storage tank.

In some embodiments, the isolation and/or selection results in one or more input compositions of enriched T cells, e.g., CD3+ T cells, CD4+ T cells, and/or CD8+ T cells. In some embodiments, two or more separate input composition are isolated, selected, enriched, or obtained from a single biological sample. In some embodiments, separate input compositions are isolated, selected, enriched, and/or obtained from separate biological samples collected, taken, and/or obtained from the same subject.

In certain embodiments, the one or more input compositions is or includes a composition of enriched T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD3+ T cells. In particular embodiment, the input composition of enriched T cells consists essentially of CD3+ T cells.

In certain embodiments, the one or more input compositions is or includes a composition of enriched CD4+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the input composition of CD4+ T cells includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells. In some embodiments, the composition of enriched T cells consists essentially of CD4+ T cells.

In certain embodiments, the one or more compositions is or includes a composition of CD8+ T cells that is or includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of CD8+ T cells contains less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free of or substantially free of CD4+ T cells. In some embodiments, the composition of enriched T cells consists essentially of CD8+ T cells.

2. Activation and Stimulation

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of stimulating or activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al.(2012) J Immunother. 35(9): 651-660, Terakura et al. (2012) Blood. 1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees Celsius, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4⁺ and/or CD8⁺ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

In some embodiments, at least a portion of the incubation in the presence of one or more stimulating conditions or a stimulatory agents is carried out in the internal cavity of a centrifugal chamber, for example, under centrifugal rotation, such as described in International Publication Number WO2016/073602. In some embodiments, at least a portion of the incubation performed in a centrifugal chamber includes mixing with a reagent or reagents to induce stimulation and/or activation. In some embodiments, cells, such as selected cells, are mixed with a stimulating condition or stimulatory agent in the centrifugal chamber. In some aspects of such processes, a volume of cells is mixed with an amount of one or more stimulating conditions or agents that is far less than is normally employed when performing similar stimulations in a cell culture plate or other system.

In some embodiments, the stimulating agent is added to cells in the cavity of the chamber in an amount that is substantially less than (e.g. is no more than 5%, 10%, 20%, 30%, 40%, 50%, 60%, 70% or 80% of the amount) as compared to the amount of the stimulating agent that is typically used or would be necessary to achieve about the same or similar efficiency of selection of the same number of cells or the same volume of cells when selection is performed without mixing in a centrifugal chamber, e.g. in a tube or bag with periodic shaking or rotation. In some embodiments, the incubation is performed with the addition of an incubation buffer to the cells and stimulating agent to achieve a target volume with incubation of the reagent of, for example, 10 mL to 200 mL, such as at least or at least about or about or 10 mL, 20 mL, 30 mL, 40 mL, 50 mL, 60 mL, 70 mL, 80 mL, 90 mL, 100 mL, 150 mL or 200 mL. In some embodiments, the incubation buffer and stimulating agent are pre-mixed before addition to the cells. In some embodiments, the incubation buffer and stimulating agent are separately added to the cells. In some embodiments, the stimulating incubation is carried out with periodic gentle mixing condition, which can aid in promoting energetically favored interactions and thereby permit the use of less overall stimulating agent while achieving stimulating and activation of cells.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, the total duration of the incubation, e.g. with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or at least about 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In particular embodiments, the stimulating conditions include incubating, culturing, and/or cultivating a composition of enriched T cells with and/or in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF).

In some embodiments, the stimulation results in activation and/or proliferation of the cells, for example, prior to transduction.

3. Vectors and Methods for Genetic Engineering

In some embodiments, engineered cells, such as T cells, used in connection with the provided methods, uses, articles of manufacture or compositions are cells have been genetically engineered to express a recombinant receptor, e.g., a CAR or a TCR described herein. In some embodiments, the cells are engineered by introduction, delivery or transfer of nucleic acid sequences that encode the recombinant receptor and/or other molecules.

In some embodiments, methods for producing engineered cells includes the introduction of a polynucleotide encoding a recombinant receptor (e.g. anti-CD19 CAR) into a cell, e.g., such as a stimulated or activated cell. In particular embodiments, the recombinant proteins are recombinant receptors, such as any described. Introduction of the nucleic acid molecules encoding the recombinant protein, such as recombinant receptor, in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation. In some embodiments, the engineering produces one or more engineered compositions of enriched T cells.

In certain embodiments, the one or more compositions of stimulated T cells are or include two separate stimulated compositions of enriched T cells. In particular embodiments, two separate compositions of enriched T cells, e.g., two separate compositions of enriched T cells that have been selected, isolated, and/or enriched from the same biological sample, are separately engineered. In certain embodiments, the two separate compositions include a composition of enriched CD4+ T cells. In particular embodiments, the two separate compositions include a composition of enriched CD8+ T cells. In some embodiments, two separate compositions of enriched CD4+ T cells and enriched CD8+ T cells are genetically engineered separately.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications. In certain embodiments, the gene transfer is accomplished by first incubating the cells under stimulating conditions, such as by any of the methods described.

In some embodiments, methods for genetic engineering are carried out by contacting one or more cells of a composition with a nucleic acid molecule encoding the recombinant protein, e.g. recombinant receptor. In some embodiments, the contacting can be effected with centrifugation, such as spinoculation (e.g. centrifugal inoculation). Such methods include any of those as described in International Publication Number WO2016/073602. Exemplary centrifugal chambers include those produced and sold by Biosafe SA, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. Patent Application, Publication No. US 2008/0171951, and published international patent application, publication no. WO 00/38762, the contents of each of which are incorporated herein by reference in their entirety. Exemplary kits for use with such systems include, but are not limited to, single-use kits sold by BioSafe SA under product names CS-430.1, CS-490.1, CS-600.1 or CS-900.2.

In some embodiments, the contacting can be effected with centrifugation, such as spinoculation (e.g., centrifugal inoculation). In some embodiments, the composition containing cells, the vector, e.g., viral particles and reagent can be rotated, generally at relatively low force or speed, such as speed lower than that used to pellet the cells, such as from or from about 600 rpm to 1700 rpm (e.g., at or about or at least 600 rpm, 1000 rpm, or 1500 rpm or 1700 rpm). In some embodiments, the rotation is carried at a force, e.g., a relative centrifugal force, of from or from about 100 g to 3200 g (e.g., at or about or at least at or about 100 g, 200 g, 300 g, 400 g, 500 g, 1000 g, 1500 g, 2000 g, 2500 g, 3000 g or 3200 g), as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured).

In some embodiments, the system is included with and/or placed into association with other instrumentation, including instrumentation to operate, automate, control and/or monitor aspects of the transduction step and one or more various other processing steps performed in the system, e.g. one or more processing steps that can be carried out with or in connection with the centrifugal chamber system as described herein or in International Publication Number WO2016/073602. This instrumentation in some embodiments is contained within a cabinet. In some embodiments, the instrumentation includes a cabinet, which includes a housing containing control circuitry, a centrifuge, a cover, motors, pumps, sensors, displays, and a user interface. An exemplary device is described in U.S. Pat. Nos. 6,123,655, 6,733,433 and US 2008/0171951.

In some embodiments, the system comprises a series of containers, e.g., bags, tubing, stopcocks, clamps, connectors, and a centrifuge chamber. In some embodiments, the containers, such as bags, include one or more containers, such as bags, containing the cells to be transduced and the viral vector particles, in the same container or separate containers, such as the same bag or separate bags. In some embodiments, the system further includes one or more containers, such as bags, containing medium, such as diluent and/or wash solution, which is pulled into the chamber and/or other components to dilute, resuspend, and/or wash components and/or compositions during the methods. The containers can be connected at one or more positions in the system, such as at a position corresponding to an input line, diluent line, wash line, waste line and/or output line.

In some embodiments, the chamber is associated with a centrifuge, which is capable of effecting rotation of the chamber, such as around its axis of rotation. Rotation may occur before, during, and/or after the incubation in connection with transduction of the cells and/or in one or more of the other processing steps. Thus, in some embodiments, one or more of the various processing steps is carried out under rotation, e.g., at a particular force. The chamber is typically capable of vertical or generally vertical rotation, such that the chamber sits vertically during centrifugation and the side wall and axis are vertical or generally vertical, with the end wall(s) horizontal or generally horizontal.

In some embodiments, during at least a part of the genetic engineering, e.g. transduction, and/or subsequent to the genetic engineering the cells are transferred to a bioreactor bag assembly for culture of the genetically engineered cells, such as for cultivation or expansion of the cells.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV) or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, the viral vector particles contain a genome derived from a retroviral genome based vector, such as derived from a lentiviral genome based vector. In some aspects of the provided viral vectors, the heterologous nucleic acid encoding a recombinant receptor, such as an antigen receptor, such as a CAR, is contained and/or located between the 5′ LTR and 3′ LTR sequences of the vector genome.

In some embodiments, the viral vector genome is a lentivirus genome, such as an HIV-1 genome or an SIV genome. For example, lentiviral vectors have been generated by multiply attenuating virulence genes, for example, the genes env, vif, vpu and nef can be deleted, making the vector safer for therapeutic purposes. Lentiviral vectors are known. See Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV). For example, lentiviral vectors have been generated by multiply attenuating the HIV virulence genes, for example, the genes env, vif, vpr, vpu and nef are deleted, making the vector safer for therapeutic purposes. Lentiviral vectors are known in the art, see Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

In some embodiments, the viral genome vector can contain sequences of the 5′ and 3′ LTRs of a retrovirus, such as a lentivirus. In some aspects, the viral genome construct may contain sequences from the 5′ and 3′ LTRs of a lentivirus, and in particular can contain the R and U5 sequences from the 5′ LTR of a lentivirus and an inactivated or self-inactivating 3′ LTR from a lentivirus. The LTR sequences can be LTR sequences from any lentivirus from any species. For example, they may be LTR sequences from HIV, SIV, FIV or BIV. Typically, the LTR sequences are HIV LTR sequences.

In some embodiments, the nucleic acid of a viral vector, such as an HIV viral vector, lacks additional transcriptional units. The vector genome can contain an inactivated or self-inactivating 3′ LTR (Zufferey et al. J Virol 72: 9873, 1998; Miyoshi et al., J Virol 72:8150, 1998). For example, deletion in the U3 region of the 3′ LTR of the nucleic acid used to produce the viral vector RNA can be used to generate self-inactivating (SIN) vectors. This deletion can then be transferred to the 5′ LTR of the proviral DNA during reverse transcription. A self-inactivating vector generally has a deletion of the enhancer and promoter sequences from the 3′ long terminal repeat (LTR), which is copied over into the 5′ LTR during vector integration. In some embodiments enough sequence can be eliminated, including the removal of a TATA box, to abolish the transcriptional activity of the LTR. This can prevent production of full-length vector RNA in transduced cells. In some aspects, the U3 element of the 3′ LTR contains a deletion of its enhancer sequence, the TATA box, Sp 1, and NF-kappa B sites. As a result of the self-inactivating 3′ LTR, the provirus that is generated following entry and reverse transcription contains an inactivated 5′ LTR. This can improve safety by reducing the risk of mobilization of the vector genome and the influence of the LTR on nearby cellular promoters. The self-inactivating 3′ LTR can be constructed by any method known in the art. In some embodiments, this does not affect vector titers or the in vitro or in vivo properties of the vector.

Optionally, the U3 sequence from the lentiviral 5′ LTR can be replaced with a promoter sequence in the viral construct, such as a heterologous promoter sequence. This can increase the titer of virus recovered from the packaging cell line. An enhancer sequence can also be included. Any enhancer/promoter combination that increases expression of the viral RNA genome in the packaging cell line may be used. In one example, the CMV enhancer/promoter sequence is used (U.S. Pat. Nos. 5,385,839 and 5,168,062).

In certain embodiments, the risk of insertional mutagenesis can be minimized by constructing the retroviral vector genome, such as lentiviral vector genome, to be integration defective. A variety of approaches can be pursued to produce a non-integrating vector genome. In some embodiments, a mutation(s) can be engineered into the integrase enzyme component of the pol gene, such that it encodes a protein with an inactive integrase. In some embodiments, the vector genome itself can be modified to prevent integration by, for example, mutating or deleting one or both attachment sites, or making the 3′ LTR-proximal polypurine tract (PPT) non-functional through deletion or modification. In some embodiments, non-genetic approaches are available; these include pharmacological agents that inhibit one or more functions of integrase. The approaches are not mutually exclusive; that is, more than one of them can be used at a time. For example, both the integrase and attachment sites can be non-functional, or the integrase and PPT site can be non-functional, or the attachment sites and PPT site can be non-functional, or all of them can be non-functional. Such methods and viral vector genomes are known and available (see Philpott and Thrasher, Human Gene Therapy 18:483, 2007; Engelman et al. J Virol 69:2729, 1995; Brown et al J Virol 73:9011 (1999); WO 2009/076524; McWilliams et al., J Virol 77:11150, 2003; Powell and Levin J Virol 70:5288, 1996).

In some embodiments, the vector contains sequences for propagation in a host cell, such as a prokaryotic host cell. In some embodiments, the nucleic acid of the viral vector contains one or more origins of replication for propagation in a prokaryotic cell, such as a bacterial cell. In some embodiments, vectors that include a prokaryotic origin of replication also may contain a gene whose expression confers a detectable or selectable marker such as drug resistance.

The viral vector genome is typically constructed in a plasmid form that can be transfected into a packaging or producer cell line. Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g., vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e. a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g., HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g., antigen receptor, such as CAR, can be detected.

In some embodiments, the provided methods involve methods of transducing cells by contacting, e.g., incubating, a cell composition comprising a plurality of cells with a viral particle.

In some embodiments, the cells to be transfected or transduced are or comprise primary cells obtained from a subject, such as cells enriched and/or selected from a subject.

In some embodiments, the concentration of cells to be transduced of the composition is from or from about 1.0×10⁵ cells/mL to 1.0×10⁸ cells/mL, such as at least or at least about or about 1.0×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL.

In some embodiments, the viral particles are provided at a certain ratio of copies of the viral vector particles or infectious units (IU) thereof, per total number of cells to be transduced (IU/cell). For example, in some embodiments, the viral particles are present during the contacting at or about or at least at or about 0.5, 1, 2, 3, 4, 5, 10, 15, 20, 30, 40, 50, or 60 IU of the viral vector particles per one of the cells.

In some embodiments, the titer of viral vector particles is between or between about 1×10⁶ IU/mL and 1×10⁸ IU/mL, such as between or between about 5×10⁶ IU/mL and 5×10⁷ IU/mL, such as at least 6×10⁶ IU/mL, 7×10⁶ IU/mL, 8×10⁶ IU/mL, 9×10⁶ IU/mL, 1×10⁷ IU/mL, 2×10⁷ IU/mL, 3×10⁷ IU/mL, 4×10⁷ IU/mL, or 5×10⁷ IU/mL.

In some embodiments, transduction can be achieved at a multiplicity of infection (MOI) of less than 100, such as generally less than 60, 50, 40, 30, 20, 10, 5 or less.

In some embodiments, the method involves contacting or incubating, the cells with the viral particles. In some embodiments, the contacting is for 30 minutes to 72 hours, such as 30 minute to 48 hours, 30 minutes to 24 hours or 1 hour to 24 hours, such as at least or at least about 30 minutes, 1 hour, 2 hours, 6 hours, 12 hours, 24 hours, 36 hours or more.

In some embodiments, contacting is performed in solution. In some embodiments, the cells and viral particles are contacted in a volume of from or from about 0.5 mL to 500 mL, such as from or from about 0.5 mL to 200 mL, 0.5 mL to 100 mL, 0.5 mL to 50 mL, 0.5 mL to 10 mL, 0.5 mL to 5 mL, 5 mL to 500 mL, 5 mL to 200 mL, 5 mL to 100 mL, 5 mL to 50 mL, 5 mL to 10 mL, 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 mL or 200 mL to 500 mL.

In certain embodiments, the input cells are treated, incubated, or contacted with particles that comprise binding molecules that bind to or recognize the recombinant receptor that is encoded by the viral DNA.

In some embodiments, the incubation of the cells with the viral vector particles results in or produces an output composition comprising cells transduced with the viral vector particles.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No. WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the anti-CD3/anti-CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

4. Cultivation, Expansion and Formulation of Engineered Cells

In some embodiments, the methods for generating the engineered cells, e.g., for cell therapy in accord with any of provided methods, uses, articles of manufacture or compositions, include one or more steps for cultivating cells, e.g., cultivating cells under conditions that promote proliferation and/or expansion. In some embodiments, cells are cultivated under conditions that promote proliferation and/or expansion subsequent to a step of genetically engineering, e.g., introducing a recombinant polypeptide to the cells by transduction or transfection. In particular embodiments, the cells are cultivated after the cells have been incubated under stimulating conditions and transduced or transfected with a recombinant polynucleotide, e.g., a polynucleotide encoding a recombinant receptor. Thus, in some embodiments, a composition of CAR-positive T cells that has been engineered by transduction or transfection with a recombinant polynucleotide encoding the CAR, is cultivated under conditions that promote proliferation and/or expansion.

In certain embodiments, the one or more compositions of engineered T cells are or include two separate compositions of enriched T cells, such as two separate compositions of enriched T cells that have been engineered with a polynucleotide encoding a recombinant receptor, e.g. a CAR. In particular embodiments, two separate compositions of enriched T cells, e.g., two separate compositions of enriched T cells selected, isolated, and/or enriched from the same biological sample, are separately cultivated under stimulating conditions, such as subsequent to a step of genetically engineering, e.g., introducing a recombinant polypeptide to the cells by transduction or transfection. In certain embodiments, the two separate compositions include a composition of enriched CD4+ T cells, such as a composition of enriched CD4+ T cells that have been engineered with a polynucleotide encoding a recombinant receptor, e.g. a CAR. In particular embodiments, the two separate compositions include a composition of enriched CD8+ T cells, such as a composition of enriched CD8+ T cells that have been engineered with a polynucleotide encoding a recombinant receptor, e.g. a CAR. In some embodiments, two separate compositions of enriched CD4+ T cells and enriched CD8+ T cells, such as a composition of enriched CD4+ T cells and a composition of enriched CD8+ T cells that have each been separately engineered with a polynucleotide encoding a recombinant receptor, e.g. a CAR, are separately cultivated, e.g., under conditions that promote proliferation and/or expansion.

In some embodiments, cultivation is carried out under conditions that promote proliferation and/or expansion. In some embodiments, such conditions may be designed to induce proliferation, expansion, activation, and/or survival of cells in the population. In particular embodiments, the stimulating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to promote growth, division, and/or expansion of the cells.

In particular embodiments, the cells are cultivated in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines, e.g. a recombinant cytokine, is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more recombinant cytokine includes IL-2, IL-7 and/or IL-15. In some embodiments, the cells, e.g., engineered cells, are cultivated in the presence of a cytokine, e.g., a recombinant human cytokine, at a concentration of between 1 IU/mL and 2,000 IU/mL, between 10 IU/mL and 100 IU/mL, between 50 IU/mL and 200 IU/mL, between 100 IU/mL and 500 IU/mL, between 100 IU/mL and 1,000 IU/mL, between 500 IU/mL and 2,000 IU/mL, or between 100 IU/mL and 1,500 IU/mL.

In some embodiments, the cultivation is performed under conditions that generally include a temperature suitable for the growth of primary immune cells, such as human T lymphocytes, for example, at least about 25 degrees Celsius, generally at least about 30 degrees Celsius, and generally at or about 37 degrees Celsius. In some embodiments, the composition of enriched T cells is incubated at a temperature of 25 to 38° C., such as 30 to 37° C., for example at or about 37° C.±2° C. In some embodiments, the incubation is carried out for a time period until the culture, e.g. cultivation or expansion, results in a desired or threshold density, number or dose of cells. In some embodiments, the incubation is greater than or greater than about or is for about or 24 hours, 48 hours, 72 hours, 96 hours, 5 days, 6 days, 7 days, 8 days, 9 days or more.

In particular embodiments, the cultivation is performed in a closed system. In certain embodiments, the cultivation is performed in a closed system under sterile conditions. In particular embodiments, the cultivation is performed in the same closed system as one or more steps of the provided systems. In some embodiments the composition of enriched T cells is removed from a closed system and placed in and/or connected to a bioreactor for the cultivation. Examples of suitable bioreactors for the cultivation include, but are not limited to, GE Xuri W25, GE Xuri W5, Sartorius BioSTAT RM 20|50, Finesse SmartRocker Bioreactor Systems, and Pall XRS Bioreactor Systems. In some embodiments, the bioreactor is used to perfuse and/or mix the cells during at least a portion of the cultivation step.

In some embodiments, the mixing is or includes rocking and/or motioning. In some cases, the bioreactor can be subject to motioning or rocking, which, in some aspects, can increase oxygen transfer. Motioning the bioreactor may include, but is not limited to rotating along a horizontal axis, rotating along a vertical axis, a rocking motion along a tilted or inclined horizontal axis of the bioreactor or any combination thereof. In some embodiments, at least a portion of the incubation is carried out with rocking. The rocking speed and rocking angle may be adjusted to achieve a desired agitation. In some embodiments the rock angle is 20°, 19°, 18°, 17°, 16°, 15°, 14°, 13°, 12°, 11°, 10°, 9°, 8°, 7°, 6°, 5°, 4°, 3°, 2° or 1°. In certain embodiments, the rock angle is between 6-16°. In other embodiments, the rock angle is between 7-16°. In other embodiments, the rock angle is between 8-12°. In some embodiments, the rock rate is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 rpm. In some embodiments, the rock rate is between 4 and 12 rpm, such as between 4 and 6 rpm, inclusive.

In some embodiments, the bioreactor maintains the temperature at or near 37° C. and CO2 levels at or near 5% with a steady air flow at, at about, or at least 0.01 L/min, 0.05 L/min, 0.1 L/min, 0.2 L/min, 0.3 L/min, 0.4 L/min, 0.5 L/min, 1.0 L/min, 1.5 L/min, or 2.0 L/min or greater than 2.0 L/min. In certain embodiments, at least a portion of the cultivation is performed with perfusion, such as with a rate of 290 ml/day, 580 ml/day, and/or 1160 ml/day, e.g., depending on the timing in relation to the start of the cultivation and/or density of the cultivated cells. In some embodiments, at least a portion of the cell culture expansion is performed with a rocking motion, such as at an angle of between 5° and 10°, such as 6°, at a constant rocking speed, such as a speed of between 5 and 15 RPM, such as 6 RMP or 10 RPM.

In some embodiments, the methods for manufacturing, generating or producing a cell therapy and/or engineered cells, in accord with the provided methods, uses or articles of manufacture, may include formulation of cells, such as formulation of genetically engineered cells resulting from the processing steps prior to or after the incubating, engineering, and cultivating, and/or one or more other processing steps as described. In some embodiments, one or more of the processing steps, including formulation of cells, can be carried out in a closed system. In some cases, the cells are processed in one or more steps (e.g. carried out in the centrifugal chamber and/or closed system) for manufacturing, generating or producing a cell therapy and/or engineered cells may include formulation of cells, such as formulation of genetically engineered cells resulting from the transduction processing steps prior to or after the culturing, e.g. cultivation and expansion, and/or one or more other processing steps as described. In some embodiments, the genetically engineered cells are formulated as unit dose form compositions including the number of cells for administration in a given dose or fraction thereof.

In some embodiments, the dose of cells comprising cells engineered with a recombinant antigen receptor, e.g. CAR or TCR, is provided as a composition or formulation, such as a pharmaceutical composition or formulation. Such compositions can be used in accord with the provided methods, such as in the treatment of diseases, conditions, and disorders, or in detection, diagnostic, and prognostic methods, and uses and articles of manufacture. In some cases, the cells can be formulated in an amount for dosage administration, such as for a single unit dosage administration or multiple dosage administration.

In some embodiments, the cells can be formulated into a container, such as a bag or vial. In some embodiments, the vial may be an infusion vial. In some embodiments, the vial is formulated with a single unit dose of the engineered cells, such as including the number of cells for administration in a given dose or fraction thereof.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the transduced and/or expanded cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the disease or condition, such as a therapeutically effective or prophylactically effective amount.

In some embodiments, the formulation buffer contains a cryopreservative. In some embodiments, the cell are formulated with a cyropreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the transduced and/or expanded cells to replace the cells in a cryopreservative solution. In some embodiments, the cells are frozen, e.g., cryoprotected or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryoprotected or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and 5%, between 0.25% and 4%, between 0.5% and 2%, or between 1% and 2% HSA.

In some embodiments, the formulation is carried out using one or more processing step including washing, diluting or concentrating the cells, such as the cultured or expanded cells. In some embodiments, the processing can include dilution or concentration of the cells to a desired concentration or number, such as unit dose form compositions including the number of cells for administration in a given dose or fraction thereof. In some embodiments, the processing steps can include a volume-reduction to thereby increase the concentration of cells as desired. In some embodiments, the processing steps can include a volume-addition to thereby decrease the concentration of cells as desired. In some embodiments, the processing includes adding a volume of a formulation buffer to transduced and/or expanded cells. In some embodiments, the volume of formulation buffer is from or from about 10 mL to 1000 mL, such as at least or at least about or about or 50 mL, 100 mL, 200 mL, 300 mL, 400 mL, 500 mL, 600 mL, 700 mL, 800 mL, 900 mL or 1000 mL.

In some embodiments, such processing steps for formulating a cell composition is carried out in a closed system. Exemplary of such processing steps can be performed using a centrifugal chamber in conjunction with one or more systems or kits associated with a cell processing system, such as a centrifugal chamber produced and sold by Biosafe SA, including those for use with the Sepax® or Sepax 2® cell processing systems. An exemplary system and process is described in International Publication Number WO2016/073602. In some embodiments, the method includes effecting expression from the internal cavity of the centrifugal chamber a formulated composition, which is the resulting composition of cells formulated in a formulation buffer, such as pharmaceutically acceptable buffer, in any of the above embodiments as described. In some embodiments, the expression of the formulated composition is to a container, such as the vials of the biomedical material vessels described herein, that is operably linked as part of a closed system with the centrifugal chamber. In some embodiments, the biomedical material vessels are configured for integration and or operable connection and/or is integrated or operably connected, to a closed system or device that carries out one or more processing steps. In some embodiments, the biomedical material vessel is connected to a system at an output line or output position. In some cases, the closed system is connected to the vial of the biomedical material vessel at the inlet tube. Exemplary close systems for use with the biomedical material vessels described herein include the Sepax® and Sepax® 2 system.

In some embodiments, the closed system, such as associated with a centrifugal chamber or cell processing system, includes a multi-port output kit containing a multi-way tubing manifold associated at each end of a tubing line with a port to which one or a plurality of containers can be connected for expression of the formulated composition. In some aspects, a desired number or plurality of vials, can be sterilely connected to one or more, generally two or more, such as at least 3, 4, 5, 6, 7, 8 or more of the ports of the multi-port output. For example, in some embodiments, one or more containers, e.g., biomedical material vessels, can be attached to the ports, or to fewer than all of the ports. Thus, in some embodiments, the system can effect expression of the output composition into a plurality of vials of the biomedical material vessels.

In some aspects, cells can be expressed to the one or more of the plurality of output containers, e.g., vials, in an amount for dosage administration, such as for a single unit dosage administration or multiple dosage administration. For example, in some embodiments, the vials, may each contain the number of cells for administration in a given dose or fraction thereof. Thus, each vial, in some aspects, may contain a single unit dose for administration or may contain a fraction of a desired dose such that more than one of the plurality of vials, such as two of the vials, or 3 of the vials, together constitute a dose for administration. In some embodiments, 4 vials together constitute a dose for administration.

Thus, the containers, e.g. bags or vials, generally contain the cells to be administered, e.g., one or more unit doses thereof. The unit dose may be an amount or number of the cells to be administered to the subject or twice the number (or more) of the cells to be administered. It may be the lowest dose or lowest possible dose of the cells that would be administered to the subject. In some aspects, the provided articles of manufacture includes one or more of the plurality of output containers.

In some embodiments, each of the containers, e.g. bags or vials, individually comprises a unit dose of the cells. Thus in some embodiments, each of the containers comprises the same or approximately or substantially the same number of cells. In some embodiments, each unit dose contains at or about or at least or at least about 1×10⁶, 2×10⁶, 5×10⁶, 1×10⁷, 5×10⁷, or 1×10⁸ engineered cells, total cells, T cells, or PBMCs. In some embodiments, each unit dose contains at or about or at least or at least about 1×10⁶, 2×10⁶, 5×10⁶, 1×10⁷, 5×10⁷, or 1×10⁸ CAR+ T cells that are CD3+, such as CD4+ or CD8+, or a viable subset thereof.

In some embodiments, the volume of the formulated cell composition in each container, e.g. bag or vial, is between at or about 10 mL and at or about 100 mL, such as at or about or at least or at least about 20 mL, 30 mL, 40 mL, 50 mL, 60 mL, 70 mL, 80 mL, 90 mL or 100 mL. In some embodiments, the volume of the formulated cell composition in each container, e.g. bag or vial, is between at or about 1 mL and at or about 10 mL, such as between at or about 1 mL and at or about 5 mL. In some embodiments, the volume of the formulated cell composition in each container, e.g. bag or vial, is between at or about 4 mL and at or about 5 mL. In some embodiments, the volume of the formulated cell composition in each container, e.g. bag or vial, is or is about 4.4 mL. In some embodiments, the volume of the formulated cell composition in each container, e.g. bag or vial, is or is about 4.5 mL. In some embodiments, the volume of the formulated cell composition in each container, e.g. bag or vial, is or is about 4.6 mL. In some embodiments, the volume of the formulated cell composition in each container, e.g. bag or vial, is or is about 4.7 mL. In some embodiments, the volume of the formulated cell composition in each container, e.g. bag or vial, is or is about 4.8 mL. In some embodiments, the volume of the formulated cell composition in each container, e.g. bag or vial, is or is about 4.9 mL. In some embodiments, the volume of the formulated cell composition in each container, e.g. bag or vial, is or is about 5.0 mL.

In some embodiments, the formulated cell composition has a concentration of greater than at or about 0.5×10⁶ recombinant receptor-expressing (e.g. CAR⁺)/CD3+ cells or such viable cells per mL, greater than at or about 1.0×10⁶ recombinant receptor-expressing (e.g. CAR⁺)/CD3+ cells or such viable cells per mL, greater than at or about 1.5×10⁶ recombinant receptor-expressing (e.g. CAR⁺)/CD3+ cells or such viable cells per mL, greater than at or about 2.0×10⁶ recombinant receptor-expressing (e.g. CAR⁺)/CD3+ cells or such viable cells per mL. greater than at or about 2.5×10⁶ recombinant receptor-expressing (e.g. CAR⁺)/CD3+ cells or such viable cells per mL, greater than at or about 2.6×10⁶ recombinant receptor-expressing (e.g. CAR⁺)/CD3+ cells or such viable cells per mL, greater than at or about 2.7×10⁶ recombinant receptor-expressing (e.g. CAR⁺)/CD3+ cells or such viable cells per mL, greater than at or about 2.8×10⁶ recombinant receptor-expressing (e.g. CAR⁺)/CD3+ cells or such viable cells per mL, greater than at or about 2.9×10⁶ recombinant receptor-expressing (e.g. CAR⁺)/CD3+ cells or such viable cells per mL greater than at or about 3.0×10⁶ recombinant receptor-expressing (e.g. CAR⁺)/CD3+ cells or such viable cells per mL, greater than at or about 3.5×10⁶ recombinant receptor-expressing (e.g. CAR⁺)/CD3+ cells or such viable cells per mL, greater than at or about 4.0×10⁶ recombinant receptor-expressing (e.g. CAR⁺)/CD3+ cells or such viable cells per mL, greater than at or about 4.5×10⁶ recombinant receptor-expressing (e.g. CAR⁺)/CD3+ cells or such viable cells per mL or greater than at or about 5×10⁶ recombinant receptor-expressing (e.g. CAR⁺)/CD3+ cells or such viable cells per mL. In some embodiments, the CD3+ cells are CD4+ T cells. In some embodiments, the CD3+ cells are CD8+ T cells. In some embodiments, the CD3+ T cells are CD4+ and CD8+ T cells.

In some embodiments, the cells in the container, e.g. bag or vials, can be cryopreserved. In some embodiments, the container, e.g. vials, can be stored in liquid nitrogen until further use.

In some embodiments, such cells produced by the method, or a composition comprising such cells, are administered to a subject for treating a disease or condition, for example, in accord with the methods, uses and articles of manufacture described herein.

### III. COMPOSITIONS AND FORMULATIONS

In some embodiments, the dose of cells comprising cells engineered with a recombinant antigen receptor, e.g. CAR or TCR, is provided as a composition or formulation, such as a pharmaceutical composition or formulation. Exemplary compositions and formulations are described above, including those produced in connection with methods of engineering the cells. Such compositions can be used in accord with the provided methods or uses, and/or with the provided articles of manufacture or compositions, such as in the prevention or treatment of diseases, conditions, and disorders, or in detection, diagnostic, and prognostic methods.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell or an agent that treats or ameliorates symptoms of neurotoxicity), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

In some embodiments, the dose of cells administered is in a cryopreserved composition. In some aspects, the composition is administered after thawing the cryopreserved composition. In some embodiments, the composition is administered within at or about 30 minutes, 45 minutes, 60 minutes, 90 minutes, 120 minutes, 150 minutes or 180 minutes after thawing. In some embodiments, the composition is administered within at or about 120 minutes after thawing.

In some embodiments, the dose of cells is administered with a syringe. In some embodiments, the syringe has a volume of at or about 0.5, 1, 2, 2.5, 3, 4, 5, 7.5, 10, 20 or 25 mL, or a range defined by any of the foregoing.

Also provided are articles of manufacture and kits containing engineered cells expressing a recombinant receptor or compositions thereof, and optionally instructions for use, for example, instructions for administering, according to the provided methods. In some embodiments, the instructions specify the criteria for selection or identification of subjects for therapy in accord with any of the provided methods.

In some embodiments, provided are articles of manufacture and/or kits that include a composition comprising a therapeutically effective amount of any of the engineered cells described herein, and instructions for administering, to a subject for treating a disease or condition. In some embodiments, the instructions can specify some or all of the elements of the methods provided herein. In some embodiments, the instructions specify particular instructions for administration of the cells for cell therapy, e.g., doses, timing, selection and/or identification of subjects for administration and conditions for administration. In some embodiments, the articles of manufacture and/or kits further include one or more additional agents for therapy, e.g., lymphodepleting therapy and/or combination therapy, such as any described herein and optionally further includes instructions for administering the additional agent for therapy. In some embodiments, the articles of manufacture and/or kits further comprise an agent for lymphodepleting therapy, and optionally further includes instructions for administering the lymphodepleting therapy. In some embodiments, the instructions can be included as a label or package insert accompanying the compositions for administration.

### IV. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, a “subject” is a mammal, such as a human or other animal, and typically is human In some embodiments, the subject, e.g., patient, to whom the agent or agents, cells, cell populations, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. In some embodiments, sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, a composition refers to any mixture of two or more products, substances, or compounds, including cells. It may be a solution, a suspension, liquid, powder, a paste, aqueous, non-aqueous or any combination thereof.

As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control or fluorescence minus one (FMO) gating control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control or fluorescence minus one (FMO) gating control under otherwise identical conditions, and/or at a level substantially lower than that for cell known to be positive for the marker, and/or at a level substantially similar as compared to that for a cell known to be negative for the marker.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### V. EXEMPLARY EMBODIMENTS

Among the provided embodiments are:

1. A method of treating indolent follicular lymphoma (FL) Grade 1, 2 or 3A, the method comprising administering to a subject having or suspected of having a disease that is relapsed/refractory (r/r) follicular lymphoma (FL) Grade 1, 2 or 3A a dose of CD4⁺ and CD8⁺ T cells, wherein T cells of the dose comprises a chimeric antigen receptor (CAR) that specifically binds to CD19, wherein:

the subject has relapsed or is refractory to treatment after at least one prior line of therapy for treating FL Grade 1, 2 or 3A, wherein at least one of the at least one prior lines of therapy includes treatment with an anti-CD20 antibody and an alkylating agent;

the dose of T cells comprises between at or about 5×10⁷ CAR-expressing T cells and at or about 1.5×10⁸ CAR-expressing T cells, inclusive;

the dose of T cells comprises a ratio of approximately 1:1 CD4⁺ T cells expressing the CAR to CD8⁺ T cells expressing the CAR; and

the administration comprises administering a plurality of separate compositions, wherein the plurality of separate compositions comprises a first composition comprising the CD8⁺ CAR-expressing T cells and a second composition comprising the CD4⁺ CAR-expressing T cells.

2. The method of embodiment 1, wherein the at least one prior line of therapy is one prior line of therapy that includes an anti-CD20 antibody and an alkylating agent.

3. The method of embodiment 2, wherein the subject has relapsed or is refractory to treatment after one prior line of therapy for treating FL Grade 1, 2 or 3A, and had progression of the disease within 24 months of initiation of the one prior line of therapy that includes an anti-CD20 antibody and an alkylating agent (POD24).

4. The method of embodiment 2 or embodiment 3, wherein the subject has relapsed or is refractory to treatment after one prior line of therapy for treating FL Grade 1, 2 or 3A, and had progression of the disease within 24 months of diagnosis after completing the one prior line of therapy that includes an anti-CD20 antibody and an alkylating agent (POD24).

5. The method of any of embodiments 1-4, wherein the at least one prior line of therapy that includes an anti-CD20 antibody and an alkylating agent is a chemoimmunotherapeutic combination therapy that includes rituximab, cyclophosphamide, vincristine, doxorubicin, and prednisolone (R-CHOP).

6. The method of an of embodiments 2-5, wherein the subject received the one prior line of therapy within six months of the original FL diagnosis.

7. The method of any of embodiments 1-6, wherein the subject has relapsed or is refractory to treatment after one prior line of therapy for treating FL Grade 1, 2 or 3A, and has at least one of the following: symptoms attributable to FL; threatened end-organ function, cytopenia secondary to lymphoma, or bulky disease; splenomegaly; and steady progression of disease over the preceding six months or more.

8. The method of any of embodiments 1-7, wherein the at least one prior line of therapy is two prior lines of therapy.

9. The method of embodiment 8, wherein the other of the two prior lines of therapy is selected from treatment with rituximab; obinutuzumab; bendamustine plus rituximab (BR); bendamustine plus obinutuzumab (BO); R-CHOP; rituximab, cyclophosphamide, vincristine, and prednisone (R-CVP); HSCT (optionally autologous or allogeneic HSCT); lenalidomide in combination with rituximab; or a PI3K inhibitor (PI3Ki), optionally wherein the PI3Ki is idelalisib, copanlisib, or duvelisib.

10. The method of any of embodiments 1-9, wherein the subject is refractory to treatment or has relapsed within 12 months of completion of a prior line of therapy, optionally wherein a prior line of therapy is a combination therapy or a monotherapy comprising a PI3Ki.

11. The method of any of embodiments 1-10, wherein the subject has relapsed after HSCT.

12. The method of any of embodiments 1-11, wherein the at least one prior line of therapy is three prior lines of therapy.

13. The method of embodiment 12, wherein the other two of the three prior lines of therapy are each independently selected from treatment with rituximab; obinutuzumab; bendamustine plus rituximab (BR); bendamustine plus obinutuzumab (BO); R-CHOP; rituximab, cyclophosphamide, vincristine, and prednisone (R-CVP); HSCT (optionally autologous or allogeneic HSCT); lenalidomide in combination with rituximab; or a PI3K inhibitor (PI3Ki), optionally wherein the PI3Ki is idelalisib, copanlisib, or duvelisib.

14. The method of any of embodiments 1-13, wherein the subject is refractory to treatment with an anti-CD20 antibody.

15. The method of any of embodiments 1-14, wherein the subject is refractory to a prior line of therapy that includes an anti-CD20 antibody and an alkylating agent.

16. The method of any of embodiments 1-15, wherein the subject relapsed within six months of completing treatment with an anti-CD20 antibody.

17. The method of any of embodiments 1-16, wherein the subject relapsed within six months of completing a prior line of therapy that includes an anti-CD20 antibody and an alkylating agent.

18. The method of any of embodiments 1-17, wherein the subject has relapsed during an anti-CD20 antibody maintenance therapy following two or more lines of therapy or within six months after completion of the anti-CD20 antibody maintenance therapy.

19. The method of any of embodiments 1-18, wherein the subject has at least one PET-positive lesion and at least one measurable nodal lesion or extranodal lesion, optionally wherein the a measurable nodal lesion is greater than 1.5 cm in the long axis regardless of the short axis and a measurable extranodal lesion is greater than 1.0 cm in the long and short axis.

20. A method of treating marginal zone lymphoma (MZL), the method comprising administering to a subject having or suspected of having relapsed/refractory (r/r) marginal zone lymphoma (MZL) a dose of CD4⁺ and CD8⁺ T cells, wherein T cells of the dose comprises a chimeric antigen receptor (CAR) that specifically binds to CD19, wherein:

the subject has relapsed or is refractory to treatment after at least two prior lines of therapy for treating MZL;

the dose of T cells comprises between at or about 5×10⁷ CAR-expressing T cells and at or about 1.5×10⁸ CAR-expressing T cells, inclusive;

the dose of T cells comprises a ratio of approximately 1:1 CD4⁺ T cells expressing the CAR to CD8⁺ T cells expressing the CAR; and

the administration comprises administering a plurality of separate compositions, wherein the plurality of separate compositions comprises a first composition comprising the CD8⁺ CAR-expressing T cells and a second composition comprising the CD4⁺ CAR-expressing T cells.

21. The method of embodiment 20, wherein the MZL is a subtype selected from among extra-nodal MZL (ENMZL, mostly gastric), splenic MZL (SMZL), and nodal MZL (NMZL).

22. The method of embodiment 20 or embodiment 21, wherein at least one of the at least two prior lines of therapy is a combination systemic therapy for treating the MZL or is a hematopoietic stem cell transplant (HSCT).

23. The method of any of embodiments 20-22, wherein at least one of the at least two prior lines of therapy is a combination systemic therapy for treating the MZL, and the combination systemic therapy is selected from rituximab plus cyclophosphamide, doxorubicin, vincristine, and prednisone (R-CHOP), or a therapy with an anti-CD20 antibody and an alkylating agent.

24. The method of any of embodiments 20-23, wherein at least one of the at least two prior lines of therapy is a combination systemic therapy that is an anti-CD20 antibody and an alkylating agent.

25. The method of any of embodiments 20-24, wherein the subject has Splenic MZL (SMZL) and at least one of the at least two prior lines of therapy is a splenectomy.

26. The method of any of embodiments 20-24, wherein the subject has extranodal MZL (ENMZL), and an antibiotic is not one of the at least two prior lines of therapy.

27. The method of any of embodiments 20-26, wherein the subject has PET non-avid disease with at least one measurable nodal lesion greater than 2.0 cm in the long axis or at least one measurable extranodal lesion.

28. The method of any of embodiments 1-27, wherein the subject does not have FL grade 3B (FL3B).

29. The method of any of embodiments 1-28, wherein the subject does not have evidence of composite DLBCL and FL, or of transformed FL (tFL).

30. The method of any of embodiments 1-29, wherein the subject does not have World Health Organization (WHO) subclassification of duodenal-type FL.

31. The method of any of embodiments 1-30, wherein the subject has relapsed to at least one prior line of therapy, and the relapse is after an initial response of complete response (CR) or partial response (PR) to the at least one prior line of therapy.

32. The method of any of embodiments 1-31, wherein the subject is refractory to at least one prior line of therapy, and the refractory treatment is a best response of stable disease (SD) or progressive disease (PD) to the at least one prior line of therapy.

33. The method of any of embodiments 1-32, wherein the subject has an Eastern Cooperative Oncology Group (ECOG) performance status of 0 or 1.

34. The method of any of embodiments 1-33, wherein the anti-CD20 antibody is a monoclonal anti-CD20 antibody.

35. The method of any of embodiments 1-34, wherein the anti-CD20 antibody is rituximab or obinutuzumab.

36. The method of any of embodiments 1-35, wherein the anti-CD20 antibody is rituximab.

37. The method of any of embodiments 1-36, wherein the alkylating agent is bendamustine or chlorambucil.

38. The method of any of embodiments 1-37, further comprising administering a lymphodepleting therapy to the subject prior to administration of the dose of CD4+ and CD8+ T cells.

39. The method of embodiment 38, wherein the lymphodepleting therapy is completed within about 7 days prior to initiation of the administration of the dose of CD4+ and CD8+ T cells.

40. The method of embodiment 38 or embodiment 39, wherein the administration of the lymphodepleting therapy is completed within about 2 to 7 days prior to initiation of the administration of the dose of CD4+ and CD8+ T cells.

41. The method of any of embodiments 38-40, wherein the lymphodepleting therapy comprises the administration of fludarabine and/or cyclophosphamide.

42. The method of any of embodiments 38-41, wherein the lymphodepleting therapy comprises administration of cyclophosphamide at or about 200-400 mg/m², optionally at or about 300 mg/m², inclusive, and/or fludarabine at or about 20-40 mg/m², optionally 30 mg/m², daily for 2-4 days, optionally for 3 days.

43. The method of any one of embodiments 38-42, wherein the lymphodepleting therapy comprises administration of cyclophosphamide at or about 300 mg/m² and fludarabine at or about 30 mg/m² daily concurrently for 3 days.

44. The method of any of embodiments 1-43, wherein CD19 is human CD19.

45. The method of any of embodiments 1-44, wherein the chimeric antigen receptor (CAR) comprises an scFv comprising the variable heavy chain region and the variable light chain region of the antibody FMC63, a spacer that is 15 amino acids of less and contains an immunoglobulin hinge region or a modified version thereof, a transmembrane domain, and an intracellular signaling domain comprising a signaling domain of a CD3-zeta (CD3ζ) chain and a costimulatory signaling region that is a signaling domain of 4-1BB.

46. The method of embodiment 45, wherein the immunoglobulin hinge region or a modified version thereof comprises the formula X₁PPX₂P, wherein X₁ is glycine, cysteine or arginine and X₂ is cysteine or threonine (SEQ ID NO:58).

47. The method of embodiment 45 or embodiment 46, wherein the immunoglobulin hinge region or a modified version thereof is an IgG1 hinge or a modified version thereof.

48. The method of embodiment 45 or embodiment 46, wherein the immunoglobulin hinge region or a modified version thereof is an IgG4 hinge or a modified version thereof.

49. The method of any of embodiments 45-48, wherein the spacer comprises the sequence set forth in SEQ ID NO: 1, SEQ ID NO: 30, SEQ ID NO: 31, SEQ ID NO: 32, SEQ ID NO: 33, or SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity to the sequence set forth in SEQ ID NO: 1, SEQ ID NO: 30, SEQ ID NO: 31, SEQ ID NO: 32, SEQ ID NO: 33, or SEQ ID NO: 34.

50. The method of any of embodiments 45-49, wherein the spacer consists of the sequence set forth in SEQ ID NO: 1, SEQ ID NO: 30, SEQ ID NO: 31, SEQ ID NO: 32, SEQ ID NO: 33, or SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity to the sequence set forth in SEQ ID NO: 1, SEQ ID NO: 30, SEQ ID NO: 31, SEQ ID NO: 32, SEQ ID NO: 33, or SEQ ID NO: 34.

51. The method of any of embodiments 45-50, wherein the spacer is at or about 12 amino acids in length.

52. The method of any of embodiments 45-51, wherein the spacer comprises the sequence set forth in SEQ ID NO: 1.

53. The method of any of embodiments 45-52, wherein the spacer consists of the sequence set forth in SEQ ID NO: 1.

54. The method of any of embodiments 45-53, wherein the transmembrane domain is a transmembrane domain of CD28.

55. The method of any of embodiments 45-54, wherein the transmembrane domain comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 8.

56. The method of any of embodiments 45-55, wherein the costimulatory domain comprises the sequence set forth in SEQ ID NO: 12 or is a variant thereof having at least 85%, 86%, 87%, 88%, 89%, 90%, 91%, 92%, 93%, 94%, 95%, 96%, 97%, 98%, 99% or more sequence identity to the sequence set forth in SEQ ID NO:12.

57. The method of any of embodiments 45-56, wherein the signaling domain of a CD3-zeta (CD3ζ) chain comprises the sequence set forth in SEQ ID NO: 13, 14, or 15, or is a variant thereof having at least 85%, 86%, 87%, 88%, 89%, 90%, 91%, 92%, 93%, 94%, 95%, 96%, 97%, 98%, 99% or more sequence identity to the sequence set forth in SEQ ID NO:13, 14, or 15.

58. The method of any of embodiments 45-57, wherein the scFv comprises a CDRL1 sequence of SEQ ID NO: 35, a CDRL2 sequence of SEQ ID NO: 55, and a CDRL3 sequence of SEQ ID NO: 56; and a CDRH1 sequence of SEQ ID NO: 38, a CDRH2 sequence of SEQ ID NO: 39, and a CDRH3 sequence of SEQ ID NO: 54.

59. The method of any of embodiments 45-57, wherein the scFv comprises a CDRL1 sequence of RASQDISKYLN (SEQ ID NO: 35), a CDRL2 sequence of SRLHSGV (SEQ ID NO: 36), and a CDRL3 sequence of GNTLPYTFG (SEQ ID NO: 37); and a CDRH1 sequence of DYGVS (SEQ ID NO: 38), a CDRH2 sequence of VIWGSETTYYNSALKS (SEQ ID NO: 39), and a CDRH3 sequence of YAMDYWG (SEQ ID NO: 40).

60. The method of any of embodiments 45-59, wherein the scFv comprises, in order from N-terminus to C-terminus, a V_{L} comprising the sequence set forth in SEQ ID NO: 42, and a V_{H}, comprising the sequence set forth in SEQ ID NO: 41.

61. The method of any of embodiments 45-60, wherein the scFv comprises the sequence set forth in SEQ ID NO:43.

62. The method of any of embodiments 1-61, wherein the CAR contains in order from N-terminus to C-terminus: an extracellular antigen-binding domain that is the scFv set forth in SEQ ID NO: 43, the spacer set forth in SEQ ID NO: 1, the transmembrane domain set forth in SEQ ID NO: 8, the 4-1BB costimulatory signaling domain set forth in SEQ ID NO: 12, and the signaling domain of a CD3-zeta (CD3ζ) chain set forth in SEQ ID NO:13.

63. The method of any of embodiments 1-62, wherein the dose of CD4+ and CD8+ T cells is between about 5×10⁷ CAR+ T cells and about 1.1×10⁸ CAR+ T cells, inclusive of each.

64. The method of any of embodiments 1-63, wherein the dose of CD4+ and CD8+ T cells is 5×10⁷CAR+ T cells.

65. The method of any of embodiments 1-63, wherein the dose of CD4+ and CD8+ T cells is 1×10⁸ CAR+ T cells.

66. The method of any of embodiments 1-65, wherein: the first composition and the second composition are administered 0 to 12 hours apart, 0 to 6 hours apart, or 0 to 2 hours apart, or wherein the administration of the first composition and the administration of the second composition are carried out on the same day, between about 0 and about 12 hours apart, between about 0 and about 6 hours apart, or between about 0 and 2 hours apart; and/or

the initiation of administration of the first composition and the initiation of administration of the second composition are carried out between about 1 minute and about 1 hour apart or between about 5 minutes and about 30 minutes apart.

67. The method of any of embodiments 1-66, wherein the first composition and the second composition are administered no more than 2 hours, no more than 1 hour, no more than 30 minutes, no more than 15 minutes, no more than 10 minutes, or no more than 5 minutes apart.

68. The method of any of embodiments 1-67, wherein the first composition and the second composition are administered less than 2 hours apart.

69. The method of any of embodiments 1-68, wherein the first composition comprising the CD8⁺ CAR-expressing T cells is administered prior to the second composition comprising the CD4⁺ CAR-expressing T cells.

70. The method of any of embodiments 1-69, wherein the dose of CD4+ T cells and CD8+ T cells is administered intravenously.

71. The method of any of embodiments 1-70, wherein the T cells are primary T cells obtained from a sample from the subject, optionally wherein the sample is a whole blood sample, an apheresis sample, or a leukapheresis sample.

72. The method of embodiment 71, wherein the sample is obtained from the subject prior to administration of the lymphodepleting therapy to the subject.

73. The method of any of embodiments 1-72, wherein the T cells are autologous to the subject.

74. The method of any of embodiments 1-73, wherein the subject is human 75. The method of any of embodiments 1-74, wherein the complete response rate (CRR) among a plurality of subjects treated according to the method is greater than at or about 50%, greater than at or about 60%, greater than at or about 70%, or greater than at or about 80%.

76. The method of embodiment 75, wherein the CRR is the percentage of subjects with a best overall response (B OR) up to 24 months of complete response (CR).

77. The method of embodiment 75 or embodiment 76, wherein the subject has FL Grade 1, 2, or 3A, and the CRR is assessed by PET-CT.

78. The method of embodiment 75 or embodiment 76, wherein the subject has MZL, and the CRR is assessed by CT.

79. Use of a dose of CD4⁺ and CD8⁺ T cells to treat a subject having or suspected of having a disease that is relapsed/refractory (r/r) follicular lymphoma (FL) Grade 1, 2 or 3A, wherein:

T cells of the dose comprise a chimeric antigen receptor (CAR) that specifically binds to CD19;

the subject has relapsed or is refractory to treatment after at least one prior line of therapy for treating FL Grade 1, 2 or 3A, wherein the at least one of the at least one prior line of therapy includes treatment with an anti-CD20 antibody and an alkylating agent;

the dose of T cells comprises between at or about 5×10⁷ CAR-expressing T cells and at or about 1.5×10⁸ CAR-expressing T cells, inclusive;

the dose of T cells comprises a ratio of approximately 1:1 CD4⁺ T cells expressing the CAR to CD8⁺ T cells expressing the CAR; and

the dose is formulated for administration as a plurality of separate compositions, wherein the plurality of separate compositions comprises a first composition comprising the CD8⁺ CAR-expressing T cells and a second composition comprising the CD4⁺ CAR-expressing T cells.

80. Use of a dose of CD4⁺ and CD8⁺ T cells in the manufacture of a medicament for the treatment of a subject having or suspected of having a disease that is relapsed/refractory (r/r) follicular lymphoma (FL) Grade 1, 2 or 3A, wherein:

T cells of the dose comprise a chimeric antigen receptor (CAR) that specifically binds to CD19;

the subject has relapsed or is refractory to treatment after at least one prior line of therapy for treating FL Grade 1, 2 or 3A, wherein at least one of the at least one prior line of therapy includes treatment with an anti-CD20 antibody and an alkylating agent;

the dose of T cells comprises between at or about 5×10⁷ CAR-expressing T cells and at or about 1.5×10⁸ CAR-expressing T cells, inclusive;

the dose of T cells comprises a ratio of approximately 1:1 CD4⁺ T cells expressing the CAR to CD8⁺ T cells expressing the CAR; and

the dose is formulated for administration as a plurality of separate compositions, wherein the plurality of separate compositions comprises a first composition comprising the CD8⁺ CAR-expressing T cells and a second composition comprising the CD4⁺ CAR-expressing T cells.

81. Use of a dose of CD4⁺ and CD8⁺ T cells to treat a subject having or suspected of having relapsed/refractory (r/r) marginal zone lymphoma (MZL), wherein:

T cells of the dose comprises a chimeric antigen receptor (CAR) that specifically binds to CD19;

the subject has relapsed or is refractory to treatment after at least two prior lines of therapy for treating MZL;

the dose of T cells comprises between at or about 5×10⁷ CAR-expressing T cells and at or about 1.5×10⁸ CAR-expressing T cells, inclusive;

the dose of T cells comprises a ratio of approximately 1:1 CD4⁺ T cells expressing the CAR to CD8⁺ T cells expressing the CAR; and

the dose is formulated for administration of a plurality of separate compositions, wherein the plurality of separate compositions comprises a first composition comprising the CD8⁺ CAR-expressing T cells and a second composition comprising the CD4⁺ CAR-expressing T cells.

82. Use of a dose of CD4⁺ and CD8⁺ T cells in the manufacture of a medicament for treatment of a subject having or suspected of having relapsed/refractory (r/r) marginal zone lymphoma (MZL), wherein:

T cells of the dose comprises a chimeric antigen receptor (CAR) that specifically binds to CD19;

the subject has relapsed or is refractory to treatment after at least two prior lines of therapy for treating MZL;

the dose of T cells comprises between at or about 5×10⁷ CAR-expressing T cells and at or about 1.5×10⁸ CAR-expressing T cells, inclusive;

the dose of T cells comprises a ratio of approximately 1:1 CD4⁺ T cells expressing the CAR to CD8⁺ T cells expressing the CAR; and

the dose is formulated for administration of a plurality of separate compositions, wherein the plurality of separate compositions comprises a first composition comprising the CD8⁺ CAR-expressing T cells and a second composition comprising the CD4⁺ CAR-expressing T cells.

### VI. EXAMPLES

The following examples are included for illustrative purposes only and are not intended to limit the scope of the invention.

### Example 1

### Analysis of Pre-Treatment Biopsy Gene Expression Profiles and Clinical Response

Therapeutic CAR⁺ T cell compositions containing autologous T cells expressing a chimeric antigen-receptor (CAR) specific for CD19 were administered to subjects with diffuse large B-cell lymphoma (DLBCL).

The therapeutic T cell compositions administered had been generated by a process including immunoaffinity-based (e g , immunomagnetic selection) enrichment of CD4⁺ and CD8⁺ cells from leukapheresis samples from the individual subjects to be treated. Isolated CD4⁺ and CD8⁺ T cells were separately activated with anti-CD3/anti-CD28 magnetic beads and independently transduced with a viral vector (e.g., lentiviral vector) encoding an anti-CD19 CAR, followed by separate expansion and cryopreservation of the engineered cell populations in a low-volume. The CAR contained an anti-CD19 scFv derived from a murine antibody (variable region derived from FMC63, V_{L}-linker-V_{H} orientation), an immunoglobulin-derived spacer, a transmembrane domain derived from CD28, a costimulatory region derived from 4-1BB, and a CD3-zeta intracellular signaling domain. The viral vector further contained sequences encoding a truncated receptor, which served as a surrogate marker for CAR expression; separated from the CAR sequence by a T2A ribosome skip sequence.

The cryopreserved cell compositions were thawed prior to intravenous administration. The therapeutic T cell dose was administered as a defined cell composition by administering a formulated CD4⁺ CAR⁺cell population and a formulated CD8⁺ CAR⁺population administered at a target ratio of approximately 1:1.

After administration of the CAR T cell composition, subjects were monitored for clinical response, including at 3 months after administration, and response to the CAR T cell composition was determined by assessing whether the subject had progressive disease (PD) or complete response (CR). Among the treated patients, 53% achieved durable CR after treatment with the CAR T cell composition, with a low incidence of severe cytokine release syndrome (CRS) and neurological events among patients with high-risk, aggressive relapsed/refractory large B-cell lymphoma. A portion of patients did not achieve CR at 1 year after CAR T cell treatment.

Tumor biopsies from an initial cohort of 50 treated subjects were collected prior to administration of the lymphodepleting chemotherapy and at approximately day 11 post-CAR T cell administration, and analyzed by RNA sequencing (RNA-seq) for gene expression. Specifically, complementary DNA (cDNA) samples were prepared from the RNA isolated from the tumor biopsies, and analyzed by RNA-seq. Samples were divided into groups of patients exhibiting either CR or PD post-treatment, while samples from subjects exhibiting stable disease (SD) or partial response (PR) were not included in analyses. Gene expression levels determined using RNA-seq from the pre-treatment tumor biopsies were correlated post facto to response following administration of the autologous therapeutic CAR T cell composition.

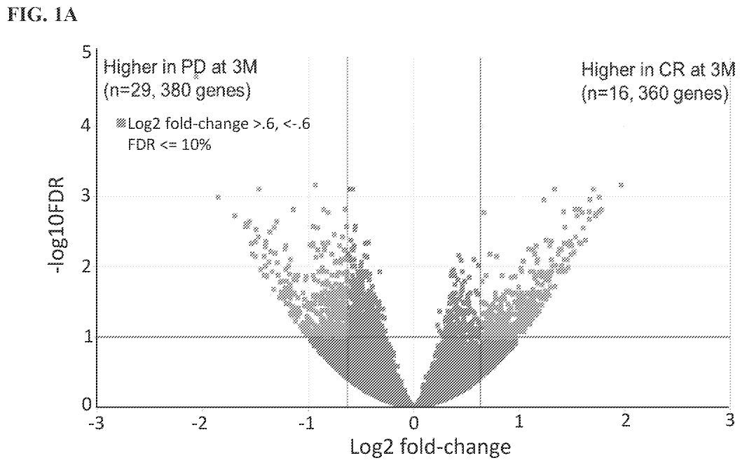

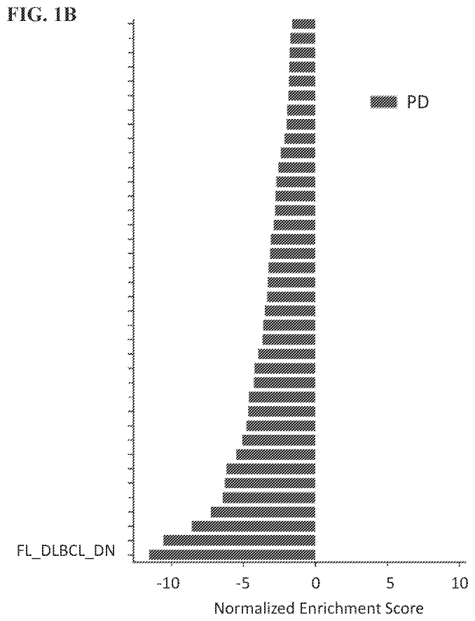

FIG. 1A shows differential gene expression profiles in pre-treatment tumor biopsies in subjects showing CR or PD at 3 months post-treatment. Setting a Loge fold-change cutoff of greater than 0.6 or less than −0.6 and a false discovery rate (FDR) of less than or equal to 10% revealed 360 genes that were highly expressed in pre-treatment biopsies from subjects showing CR at 3 months post-treatment (n=16) and 380 genes that were highly expressed in pre-treatment biopsies from subjects with PD at 3 months post-treatment (n=29). Of the differentially expressed genes, expression of genes associated with T cells was higher in pre-treatment biopsies from subjects that exhibited CR at 3 months post-treatment. Expression of EZH2, a gene encoding histone lysine methyltransferase enhancer of zeste homolog 2, and genes that are targets of EZH2 was higher in pre-treatment biopsies for subjects that showed PD at 3 months post-treatment.

As shown in FIG. 1B, a gene set identified as genes that were expressed at a higher level in DLBCL compared to FL (designated “FL_DLBCL_DN” gene set; from an analysis of differential gene expression in 75 available DLBCL cell line samples and 75 available FL cell line samples as described in Example 2 below) was also found to be highly enriched for genes associated with biopsies from subjects exhibiting PD 3 months post-treatment.

In an analysis of a larger cohort having an additional 24 patients from the same study (74 total subjects; “larger cohort”), similar results were observed. Similar to above, in this set of subjects, tumor biopsy samples were taken prior to treatment for 74 subjects and approximately 11 days post-treatment for 56 subjects, and matched biopsies were taken pre-treatment and at approximately 11 days post-treatment for 28 of these subjects, for RNA-seq analysis as described above. Among the larger cohort, 46% of patients with pre-treatment biopsies exhibited CR at 3 months post-treatment, and 56% of patients with day 11 biopsies exhibited CR at 3 months post-treatment. To determine whether there was a bias in response outcomes of the RNA-seq study population compared to the population of subjects for whom an RNA-seq sample was not obtained, CR, PD, partial response (PR), and progression free survival (PFS) outcomes were compared between the two populations. No significant differences were observed.

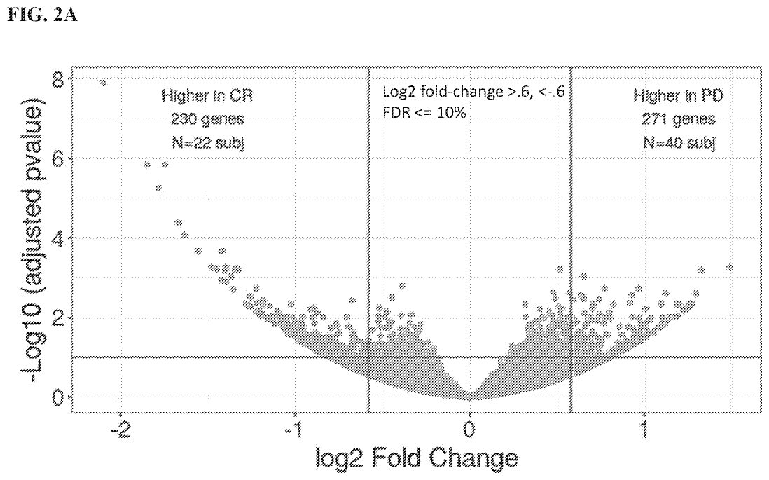

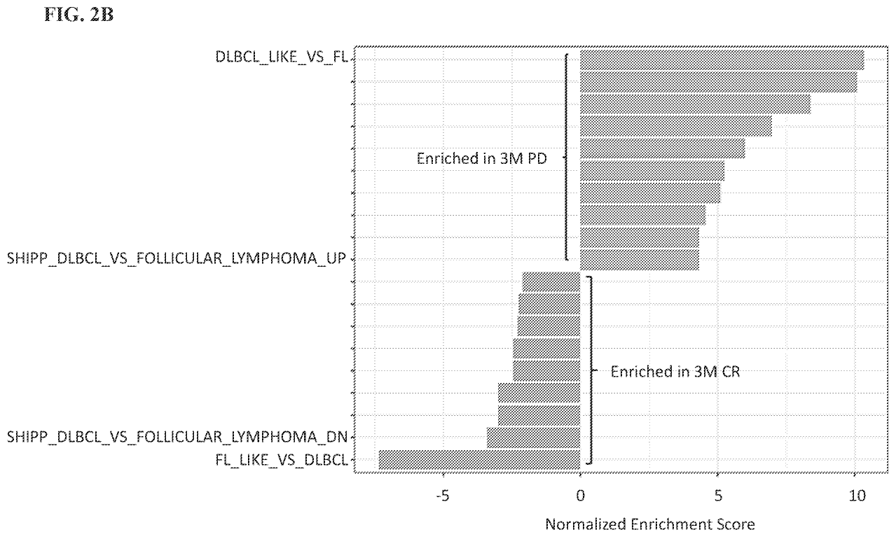

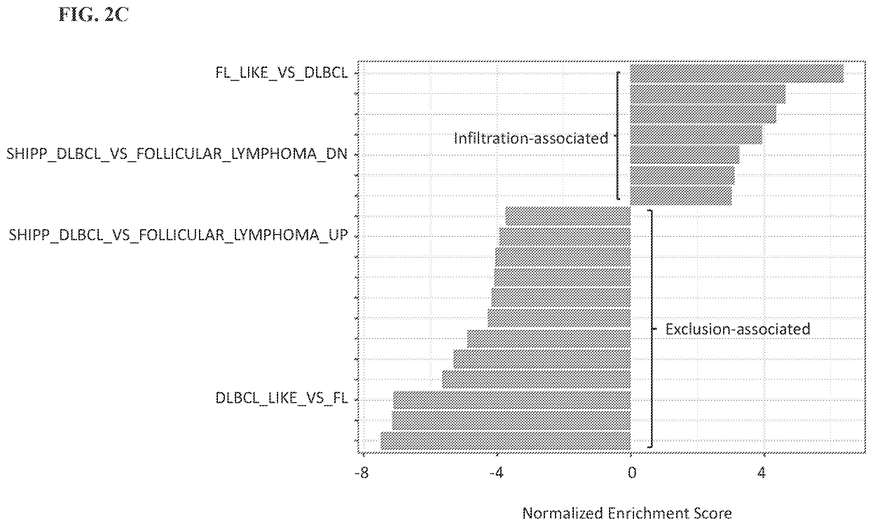

Differential gene expression analysis was also performed on the pre-treatment tumor biopsies of the larger cohort to identify genes more highly expressed in subjects exhibiting CR at 3 months post-treatment and genes more highly expressed in subjects exhibiting PD at 3 months post-treatment. In this group of subjects, setting a Log2 fold-change cutoff of greater than 0.6 or less than -0.6 and an FDR rate of less than or equal to 10% revealed 230 genes that were more highly expressed in pre-treatment biopsies from subjects showing CR at 3 months post-treatment (n=22) and 271 genes that were more highly expressed in pre-treatment biopsies from subjects showing PD at 3 months post-treatment (n=40) (FIG. 2A).

In a further analysis of the larger cohort, and as shown in FIG. 2B, gene expression analysis of pre-treatment tumor biopsies revealed that gene sets comprising genes expressed more highly in DLBCL compared to FL (DLBCL_LIKE_vs_FL) and the most upregulated genes distinguishing DLBCL from FL (SHIPP_DLBCL_VS_FOLLICULAR_LYMPHOMA_UP) were enriched in patients exhibiting PD at 3 months post-treatment. By contrast, genes sets comprising genes expressed more highly in FL compared to DLBCL (FL_LIKE_vs_DLBCL) and the most downregulated genes distinguishing DLBCL from FL (SHIPP_DLBCL_VS_FOLLICULAR_LYMPHOMA_DN) were enriched in patients exhibiting CR at 3 months post-treatment.

These results support that genes expressed at a higher level in DLBCL compared to FL, and EZH2 target genes, were enriched in the pre-treatment biopsy samples among genes associated with PD at 3 months in subjects following administration of CAR-T cells.

For the 28 matched pre-treatment and day 11 post-treatment samples described above, immune infiltration at day 11 post-treatment was estimated using the average of several T-cell genes, including the CAR transcript. The median of this infiltration level was used to split the subjects into two groups—those with high infiltration (n=14) and those with low infiltration (n=14). Differential gene expression analysis was conducted on the 28 pre-treatment samples grouped by their matching day 11 “high” or “low” infiltration levels to identify gene expression in pre-treatment biopsies that was associated with T-cell infiltration following administration of anti-CD19 CAR T cells. As shown in FIG. 2C, subjects in the “high” infiltration group exhibited enrichment of gene sets comprising genes expressed more highly in FL compared to DLBCL (FL_LIKE_vs_DLBCL) and the most downregulated genes distinguishing DLBCL from FL (SHIPP_DLBCL_VS_FOLLICULAR_LYMPHOMA_DN). Among subjects in the “low” infiltration group, enrichment of gene sets comprising genes expressed more highly in DLBCL compared to FL (DLBCL_LIKE_vs_FL) and the most upregulated genes distinguishing DLBCL from FL (SHIPP_DLBCL_VS_FOLLICULAR_LYMPHOMA_UP) was observed.

These data indicate that FL-like gene expression is associated with increased T cell infiltration, as well as improved response to treatment.

### Example 2

### Gene Expression Analysis of Diffuse Large B-Cell Lymphoma (DLBCL) vs. Follicular Lymphoma (FL)

Studies were carried out to explore biology differences between diffuse large B-cell lymphoma (DLBCL) and follicular lymphoma (FL) using an internal dataset.

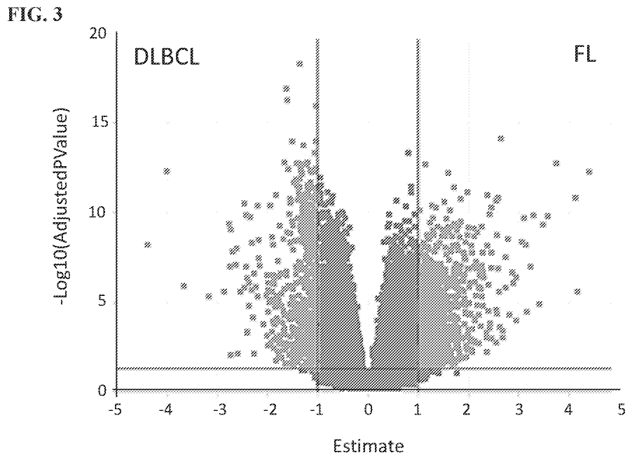

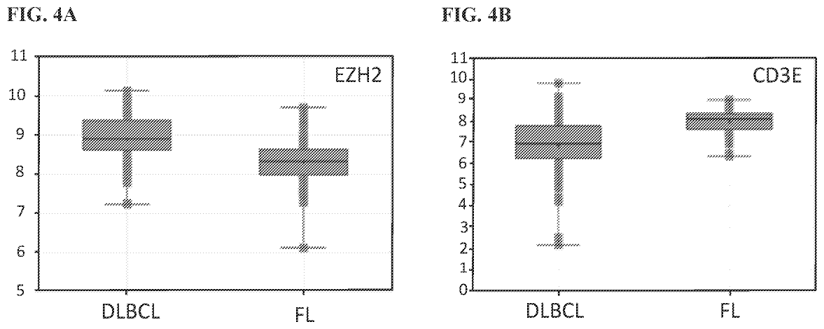

RNA-seq for gene expression was carried out on the complementary DNA (cDNA) samples prepared from RNA isolated from about 75 Follicular Lymphoma (FL) and about 75 diffuse large B-cell lymphoma (DLBCL) available tumor cell samples (formalin-fixed paraffin-embedded tumor biopsies), described above in Example 1. The tumor cell samples analyzed harbored either wild-type or mutated EZH2, and included both germinal center B-cell-like (GCB) and activated B-cell (ABC) subtypes of DLBCL. As shown in FIG. 3, differential gene expression was observed in DLBCL and FL tumor cell samples with different sets of genes elevated in the DLBCL (“DLBCL-like”) and FL (“FL-like”) tumor cell samples. Among the genes that exhibited differential expression between FL and DLBCL tumor cell samples were EZH2 (FIG. 4A; encoding enhancer of zeste homolog 2, a histone lysine methyltransferase) and CD3E (FIG. 4B; encoding T-cell surface glycoprotein CD3 epsilon chain, the epsilon polypeptide of the CD3-TCR complex). As shown, FL tumor cell samples showed lower expression levels of EZH2 and higher expression levels of CD3E compared to DLBCL tumor cell samples.

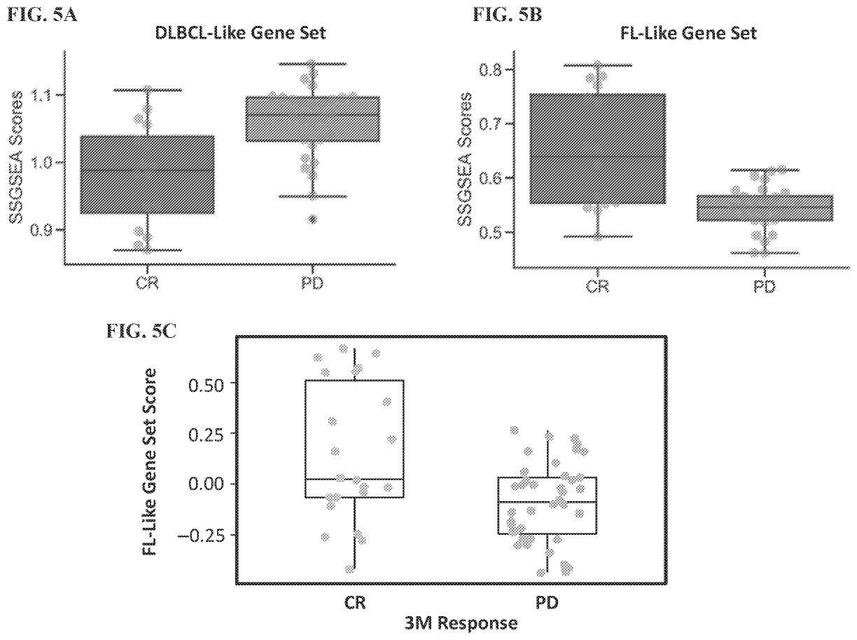

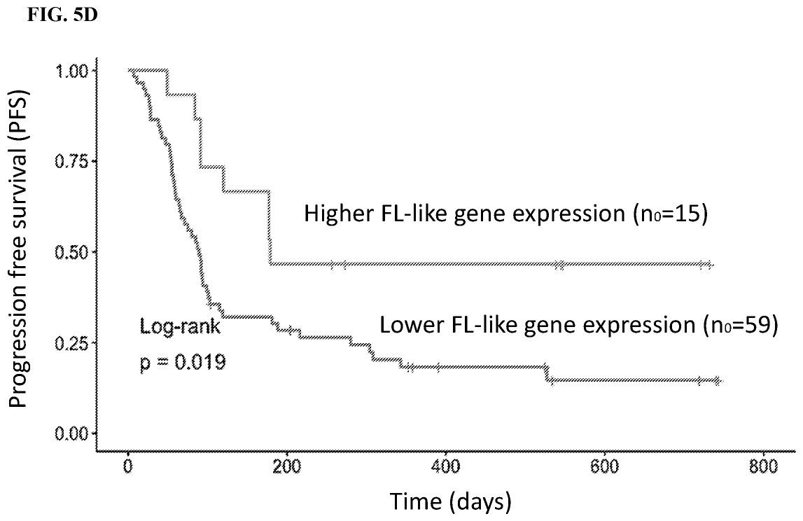

Gene expression profiles by RNA-seq on pre-treatment tumor biopsies, from the initial cohort of subjects described in Example 1 who then were administered the anti-CD19 therapeutic T cell composition as described, were analyzed for expression of genes from the study described in FIG. 3 that were found to be elevated in DLBCL compared to FL (designated “DLBCL-like” or “DLBCL_LIKE_vs_FL” gene set), or elevated in FL compared to DLBCL (designated “FL-like” or “FL_LIKE_vs_DLBCL” gene set). A single-sample Gene Set Enrichment Analysis (ssGSEA) was carried out to calculate separate enrichment scores for each pairing of a sample and the respective gene set. As shown in FIG. 5A, subjects who went on to exhibit CR in the study described in Example 1 had a lower ssGSEA score for the DLBCL-like gene set (DLBCL_LIKE_vs_FL) compared to subjects who went on to exhibit PD. Conversely, as shown in FIG. 5B, subjects who went on to exhibit CR in the study described in Example 1 had a higher ssGSEA score for the FL-like gene set (FL_LIKE_vs_DLBCL) compared to subjects who went on to exhibit PD.

In a similar analysis among subjects in the larger cohort described in Example 1, similar results were observed. Subjects who exhibited CR at 3 months-post treatment had significantly higher enrichment scores for the FL-like gene set (FIG. 5C; n=62). In addition, eight of the patients had distinctly “FL-like” gene expression scores; these subjects with the highest “FL-like” gene expression showed a high rate of CR at 3 months post-treatment (CR=100%) compared to non-“FL-like” subjects (CR=22%). Several of the DLBCL subjects with a high FL-like score had transformed follicular lymphoma (tFL) histology, but there were also other tFL subjects with a low FL-like score. Similarly, some non-tFL subjects had a high FL-like score. Among subjects in the larger cohort, progression free survival (PFS) curves were compared for the 15 subjects with the highest FL-like scores and the remaining 59 subjects, with subjects having high FL-like gene expression exhibiting significantly higher PFS (FIG. 5D).

These data demonstrate that the gene expression profiles of subjects with DLBCL and FL are different. Specifically, gene expression differences between DLBCL and FL tumor biopsies are related to the amount of immune or stromal content or the tumor cell state. These data are consistent with an observation that subjects with FL or subjects with higher expression of genes found to be elevated in FL subjects, compared to subjects with DLBCL or subjects with higher expression of genes found to be elevated in DLBCL subjects, may be less resistant to T cell infiltration into the tumor environment. Further, subjects with DLBCL tumors that appear more similar to FL tumors may have improved PFS outcomes following CAR T cell treatment.

### Example 3

### Administration of Anti-CD19 CAR-Expressing Cells to Subjects with Follicular Lymphoma (FL) or Marginal Zonal Lymphoma (MZL)

An anti-CD19 CAR-expressing therapeutic T cell composition containing a defined composition (˜1:1 ratio) of CD4+ CAR-expressing T cell compositions and CD8+ CAR-expressing T cell compositions was administered to subjects with relapsed/refractory (R/R) indolent B cell non-Hodgkin lymphoma (NHL), including indolent follicular lymphoma (FL) Grade 1-3A and marginal zone lymphoma (MZL).

Subjects were divided into four cohorts as follows: Cohort 1, fourth line and beyond (4L+) r/r/FL, including double refractory subjects; Cohort 2, third line (3L) r/r FL, including double refractory subjects; Cohort 3, second line (2L) r/r FL, including subjects with progression of disease within 24 months of diagnosis and/or initiation of treatment (POD24); and Cohort 4, third line and beyond (3L+) MZL. Thus, a group of subjects with high-risk features, including double refractory subjects and subjects with progression of disease within 24 months of diagnosis (POD24) were enrolled. Double refractory subjects were those who met at least one of the following criteria: (i) are refractory to treatment with a prior therapy that included an anti-CD20 antibody and an alkylating agent (e.g. bendamustine), (ii) have relapsed within 6 months (i.e. during or up to 6 months) after completing treatment of a prior therapy that included an anti-CD20 monoclonal antibody and an alkylating agent (e.g. bendamustine), or (iii) have relapsed during anti-CD20 maintenance following 2 or more lines of therapy or within 6 months after maintenance completion. POD24 subjects were those that had progressed within 24 months of diagnosis or initiation of treatment after treatment with a chemoimmunotherapeutic combination therapy, such as an anti-CD20 antibody and an alkylating agent (e.g. bendamustine). Other high risk features included relapse within 12 months of completion of prior combination therapy, and failure after hematopoietic stem cell transplant. Subjects with grade 3B FL (FL3B) were excluded from enrollment. In addition, subjects with evidence of composite DLBCL and FL, or of transformed FL also were excluded from enrollment. All subjects had an Eastern Cooperative Oncology Group performance (ECOG) status of 0 or 1. Exemplary cohorts are set forth in Table E1.

TABLE E1
Exemplary Cohorts

| Cohort | Description |
|---|---|
| Cohort 1 | Subjects have received at least 3 prior lines of systemic therapy wherein at least one of these |
| (4L + r/r FL) | lines was a combination which included an anti-CD20 antibody (eg, rituximab, obinutuzumab) |
| | and an alkylating agent. Prior HSCT is permitted as a prior line of therapy. |
| | In addition to these requirements, a group of 4L + double-refractory subjects must also meet one |
| | of the following criteria: (i) are refractory to a systemic line of therapy which included an anti- |
| | CD20 antibody and an alkylating agent, (ii) have relapsed within 6 months after completion of a |
| | prior line of systemic therapy which included an anti-CD20 antibody and an alkylating agent, |
| | and/or (iii) have relapsed during anti-CD20 antibody maintenance following 2 or more lines of |
| | therapy or within 6 months after maintenance completion |
| Cohort 2 | Subjects have received 2 prior lines of systemic therapy wherein at least one of these lines was a |
| (3L r/r FL) | combination which included an anti-CD20 antibody (eg, rituximab, obinutuzumab) and an |
| | alkylating agent. |
| | In addition to this requirement, subjects must meet one of the following high-risk criteria, (i) |
| | have relapsed or refractory disease within 12 months of completion of a prior line of therapy and |
| | have received prior combination therapies. Monotherapy with a PI3Ki is an accepted line of |
| | therapy, (ii) have relapsed after HSCT, and/or (iii) meet the definition of double refractory. |
| | 3L double refractory subjects must meet one of the following criteria: (i) are refractory to a |
| | systemic line of therapy which included an anti-CD20 antibody and an alkylating agent, (ii) have |
| | relapsed within 6 months after completion of a prior line of systemic therapy which included an |
| | anti-CD20 antibody and an alkylating agent, and/or (iii) have relapsed during anti-CD20 |
| | antibody maintenance following 2 or more lines of therapy or within 6 months after maintenance |
| | completion |
| Cohort 3 | Subjects have received 1 prior line of combination systemic therapy, which included an anti- |
| (2L r/r FL) | CD20 antibody (eg, rituximab, obinutuzumab) and an alkylating agent. |
| | A group of POD24 subjects, defined as having progressive disease within 24 months of |
| | diagnosis and/or initiation of treatment,and have received treatment within 6 months of the |
| | original FL diagnosis, will be enrolled to this cohort |
| | The 2L subjects that do not meet this POD24 definition must instead meet one of the modified |
| | GELF* criteria: (i) have symptoms attributable to FL (not limited to B symptoms), (ii) have |
| | threatened end-organ function, or cytopenia secondary to lymphoma, or bulky disease (single |
| | mass > 7 cm or 3 or more masses > 3 cm), (iii) splenomegaly, and/or (iv) steady progression |
| | over at least 6 months. |
| Cohort 4 | Subjects have received at least 2 prior systemic therapies, wherein at least one line of therapy |
| (3L + MZL) | was a combination systemic therapy, therapy with an anti-CD20 antibody (eg, rituximab, |
| | obinutuzumab) and an alkylating agent, or relapsed after HSCT. Splenectomy for Splenic MZL |
| | (SMZL) is considered as a line of therapy. Antibiotics for extranodal MZL (ENMZL) are not |
| | considered as a prior line of therapy. |

*National Comprehensive Cancer Network. NCCN Clinical Practice Guidelines in Oncology (NCCN Guidelines): B-cell Lymphomas; V4. 2019

The anti-CD19 CAR-expressing therapeutic T cell compositions administered were generated by a process including immunoaffinity-based (e.g., immunomagnetic selection) enrichment of CD4⁺ and CD8⁺ cells from leukapheresis samples from the individual subjects treated. Isolated CD4⁺ and CD8⁺ T cells were separately activated and independently transduced with a viral vector (e.g., lentiviral vector) encoding an anti-CD19 CAR, followed by separate expansion and cryopreservation of the engineered cell populations in a low-volume. The CAR contained an anti-CD19 scFv derived from a murine antibody (variable region derived from FMC63, V_{L}-linker-V_{H} orientation), an immunoglobulin-derived spacer, a transmembrane domain derived from CD28, a costimulatory region derived from 4-1BB, and a CD3-zeta intracellular signaling domain. The viral vector further contained sequences encoding a truncated receptor separated from the CAR sequence by a T2A ribosome skip sequence, which served as a surrogate marker for CAR expression.

The CD4+ and CD8+ cryopreserved cell compositions were thawed prior to intravenous administration. The therapeutic T cell dose was administered as a defined cell composition by administering a formulated CD4⁺ CAR⁺ cell population and a formulated CD8⁺ CAR⁺ population administered at a target ratio of approximately 1:1.

Prior to CAR+ T cell infusion, subjects received a lymphodepleting chemotherapy with fludarabine (flu, 30 mg/m²/day) and cyclophosphamide (Cy, 300mg/m²/day) for three (3) days. In some cases, prior to administration of the lymphodepleting chemotherapy, PET-positive disease was re-confirmed for FL subjects and CT-positive disease was re-confirmed for MZL subjects.

The subjects received CAR-expressing T cells 2-7 days after lymphodepletion. Subjects were administered a single dose of 1×10⁸ (100×10⁶) CAR-expressing T cells (each single dose via separate infusions at a 1:1 ratio of CD4+ CAR-expressing T cells and CD8+ CAR-expressing T cells, respectively).

Response to treatment was assessed based on radiographic tumor assessment by positron emission tomography (PET) and/or computed tomography (CT) and/or magnetic resonance imaging (MRI) scans at baseline prior to treatment and at various times following treatment (e.g. based on Lugano classification, see, e.g., Cheson et al., (2014) JCO 32(27):3059-3067). FL subjects were primarily assessed using PET scans, whereas MZL subjects were primarily assessed using CT/MRI scans. Response outcomes assessed also included complete response rate (CRR) and overall response rate (ORR). In some cases, duration of response (DOR), progression-free survival (PFS), and overall survival (OS) were also assessed.

Safety evaluations were also monitored and included adverse event (AE)/serious adverse event (SAE) collection, concomitant medication and procedure assessment, laboratory evaluations, physical examinations and vital sign assessment. AEs and laboratory abnormalities (type, frequency and severity) were collected, graded and reported according to National Cancer Institute Common Terminology Criteria for Adverse Events (NCI CTCAE version 5.0). See U.S. Department of Health and Human Services, Published: Nov. 27, 017. Adverse events of special interest (AESIs) included but were not limited to cytokine release syndrome (CRS), neurotoxicity (NT), infusion reaction, macrophage activation syndrome (MAS), tumor lysis syndrome (TLS), hypogammaglobulinemia, prolonged cytopenia, infections, second primary malignancy (SPM) and autoimmune events. Cytokine release syndrome (CRS) and neurotoxicity (NT) were graded according to the American Society for Transplantation and Cellular Therapy (ASTCT) Consensus Grading System (Lee et al. Biol Blood Marrow Transplant. 2019 April; 25(4):625-38). Signs and symptoms of CRS and neurotoxicity were also monitored.

Other endpoints assessed included pharmacokinetics (PK), including maximum concentration (Cₘₐₓ), time to maximum concentration (Tₘₐₓ), area under the curve (AUC), and persistence of the CAR-expressing T cells (e.g. as assessed by PCR); pharmacodynamics, including measurement of peripheral B-cell aplasia; health-related quality of life (HRQoL), as assessed by EORTC QLQ-C30 and/or FACT-LymS.

In some cases, response, safety, and other endpoints were evaluated for at least or up to about 24 months following treatment.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | ESKYGPPCPPCP | spacer (IgG4hinge) |
| | | (aa) |
| 2 | GAATCTAAGTACGGACCGCCCTGCCCCCCTTGCCCT | spacer (IgG4hinge) |
| | | (nt) |
| 3 | ESKYGPPCPPCPGQPREPQVYTLPPSQEEMTKNQVSLTCLVKGFYPSDIA | Hinge-CH3 spacer |
| | VEWESNGQPENNYKTTPPVLDSDGSFFLYSRLTVDKSRWQEGNVFSCSVM | |
| | HEALHNHYTQKSLSLSLGK | |
| 4 | ESKYGPPCPPCPAPEFLGGPSVFLFPPKPKDTLMISRTPEVTCVVVDVSQ | Hinge-CH2-CH3 |
| | EDPEVQFNWYVDGVEVHNAKTKPREEQFNSTYRVVSVLTVLHQDWLNGKE | spacer |
| | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGK | |
| 5 | RWPESPKAQASSVPTAQPQAEGSLAKATTAPATTRNTGRGGEEKKKEKEK | IgD-hinge-Fc |
| | EEQEERETKTPECPSHTQPLGVYLLTPAVQDLWLRDKATFTCFVVGSDLK | |
| | DAHLTWEVAGKVPTGGVEEGLLERHSNGSQSQHSRLTLPRSLWNAGTSVT | |
| | CTLNHPSLPPQRLMALREPAAQAPVKLSLNLLASSDPPEAASWLLCEVSG | |
| | FSPPNILLMWLEDQREVNTSGFAPARPPPQPGSTTFWAWSVLRVPAPPSP | |
| | QPATYTCWSHEDSRTLLNASRSLEVSYVTDH | |
| 6 | LEGGGEGRGSLLTCGDVEENPGPR | T2A |
| 7 | MLLLVTSLLLCELPHPAFLLIPRKVCNGIGIGEFKDSLSINATNIKHFKN | tEGFR |
| | CTSISGDLHILPVAFRGDSFTHTPPLDPQELDILKTVKEITGFLLIQAWP | |
| | ENRTDLHAFENLEIIRGRTKQHGQFSLAVVSLNITSLGLRSLKEISDGDV | |
| | IISGNKNLCYANTINWKKLFGTSGQKTKIISNRGENSCKATGQVCHALCS | |
| | PEGCWGPEPRDCVSCRNVSRGRECVDKCNLLEGEPREFVENSECIQCHPE | |
| | CLPQAMNITCTGRGPDNCIQCAHYIDGPHCVKTCPAGVMGENNTLVWKYA | |
| | DAGHVCHLCHPNCTYGCTGPGLEGCPTNGPKIPSIATGMVGALLLLLVVA | |
| | LGIGLFM | |
| 8 | FWVLVVVGGVLACYSLLVTVAFIIFWV | CD28 (amino acids |
| | | 153-179 of |
| | | Accession No. |
| | | P10747) |
| 9 | IEVMYPPPYLDNEKSNGTIIHVKGKHLCPSPLFPGPSKPFWVLVVVGGVL | CD28 (amino acids |
| | ACYSLLVTVAFIIFWV | 114-179 of |
| | | Accession No. |
| | | P10747) |
| 10 | RSKRSRLLHSDYMNMTPRRPGPTRKHYQPYAPPRDFAAYRS | CD28 (amino acids |
| | | 180-220 of P10747) |
| 11 | RSKRSRGGHSDYMNMTPRRPGPTRKHYQPYAPPRDFAAYRS | CD28 (LL to GG) |
| 12 | KRGRKKLLYIFKQPFMRPVQTTQEEDGCSCRFPEEEEGGCEL | 4-1BB (amino acids |
| | | 214-255 of |
| | | Q07011.1) |
| 13 | RVKFSRSADAPAYQQGQNQLYNELNLGRREEYDVLDKRRGRDPEMGGKPR | CD3 zeta |
| | RKNPQEGLYNELQKDKMAEAYSEIGMKGERRRGKGHDGLYQGLSTATKDT | |
| | YDALHMQALPPR | |
| 14 | RVKFSRSAEPPAYQQGQNQLYNELNLGRREEYDVLDKRRGRDPEMGGKPR | CD3 zeta |
| | RKNPQEGLYNELQKDKMAEAYSEIGMKGERRRGKGHDGLYQGLSTATKDT | |
| | YDALHMQALPPR | |
| 15 | RVKFSRSADAPAYKQGQNQLYNELNLGRREEYDVLDKRRGRDPEMGGKPR | CD3 zeta |
| | RKNPQEGLYNELQKDKMAEAYSEIGMKGERRRGKGHDGLYQGLSTATKDT | |
| | YDALHMQALPPR | |
| 16 | RKVCNGIGIGEFKDSLSINATNIKHFKNCTSISGDLHILPVAFRGDSFTH | tEGFR |
| | TPPLDPQELDILKTVKEITGFLLIQAWPENRTDLHAFENLEIIRGRTKQH | |
| | GQFSLAVVSLNITSLGLRSLKEISDGDVIISGNKNLCYANTINWKKLFGT | |
| | SGQKTKIISNRGENSCKATGQVCHALCSPEGCWGPEPRDCVSCRNVSRGR | |
| | ECVDKCNLLEGEPREFVENSECIQCHPECLPQAMNITCTGRGPDNCIQCA | |
| | HYIDGPHCVKTCPAGVMGENNTLVWKYADAGHVCHLCHPNCTYGCTGPGL | |
| | EGCPTNGPKIPSIATGMVGALLLLLVVALGIGLFM | |
| 17 | EGRGSLLTCGDVEENPGP | T2A |
| 18 | GSGATNFSLLKQAGDVEENPGP | P2A |
| 19 | ATNFSLLKQAGDVEENPGP | P2A |
| 20 | QCTNYALLKLAGDVESNPGP | E2A |
| 21 | VKQTLNFDLLKLAGDVESNPGP | F2A |
| 22 | -PGGG-(SGGGG)₅-P- wherein P is proline, G is | Linker |
| | glycine and S is serine | |
| 23 | GSADDAKKDAAKKDGKS | Linker |
| 24 | atgcttctcctggtgacaagccttctgctctgtgagttaccacacccagc | GMCSFR alpha |
| | attcctcctgatccca | chain signal |
| | | sequence |
| 25 | MLLLVTSLLLCELPHPAFLLIP | GMCSFR alpha |
| | | chain signal |
| | | sequence |
| 26 | MALPVTALLLPLALLLHA | CD8 alpha signal |
| | | peptide |
| 27 | Glu Pro Lys Ser Cys Asp Lys Thr His Thr Cys Pro | Hinge |
| | Pro Cys Pro | |
| 28 | Glu Arg Lys Cys Cys Val Glu Cys Pro Pro Cys Pro | Hinge |
| 29 | ELKTPLGDTHTCPRCPEPKSCDTPPPCPRCPEPKSCDTPPPCPRCPEPKS | Hinge |
| | CDTPPPCPRCP | |
| 30 | Glu Ser Lys Tyr Gly Pro Pro Cys Pro Ser Cys Pro | Hinge |
| 31 | Glu Ser Lys Tyr Gly Pro Pro Cys Pro Pro Cys Pro | Hinge |
| 32 | Tyr Gly Pro Pro Cys Pro Pro Cys Pro | Hinge |
| 33 | Lys Tyr Gly Pro Pro Cys Pro Pro Cys Pro | Hinge |
| 34 | Glu Val Val Val Lys Tyr Gly Pro Pro Cys Pro Pro | Hinge |
| | Cys Pro | |
| 35 | RASQDISKYLN | CDR L1 |
| 36 | SRLHSGV | CDR L2 |
| 37 | GNTLPYTFG | CDR L3 |
| 38 | DYGVS | CDR H1 |
| 39 | VIWGSETTYYNSALKS | CDR H2 |
| 40 | YAMDYWG | CDR H3 |
| 41 | EVKLQESGPGLVAPSQSLSVTCTVSGVSLPDYGVSWIRQPPRKGLEWLGV | VH |
| | IWGSETTYYNSALKSRLTIIKDNSKSQVFLKMNSLQTDDTAIYYCAKHYY | |
| | YGGSYAMDYWGQGTSVTVSS | |
| 42 | DIQMTQTTSSLSASLGDRVTISCRASQDISKYLNWYQQKPDGTVKLLIYH | VL |
| | TSRLHSGVPSRFSGSGSGTDYSLTISNLEQEDIATYFCQQGNTLPYTFGG | |
| | GTKLEIT | |
| 43 | DIQMTQTTSSLSASLGDRVTISCRASQDISKYLNWYQQKPDGTVKLLIYH | scFv |
| | TSRLHSGVPSRFSGSGSGTDYSLTISNLEQEDIATYFCQQGNTLPYTFGG | |
| | GTKLEITGSTSGSGKPGSGEGSTKGEVKLQESGPGLVAPSQSLSVTCTVS | |
| | GVSLPDYGVSWIRQPPRKGLEWLGVIWGSETTYYNSALKSRLTIIKDNSK | |
| | SQVFLKMNSLQTDDTAIYYCAKHYYYGGSYAMDYWGQGTSVTVSS | |
| 44 | KASQNVGTNVA | CDR L1 |
| 45 | SATYRNS | CDR L2 |
| 46 | QQYNRYPYT | CDR L3 |
| 47 | SYWMN | CDR H1 |
| 48 | QIYPGDGDTNYNGKFKG | CDR H2 |
| 49 | KTISSWDFYFDY | CDR H3 |
| 50 | EVKLQQSGAELVRPGSSVKISCKASGYAFSSYWMNWVKQRPGQGLEWIGQ | VH |
| | IYPGDGDTNYNGKFKGQATLTADKSSSTAYMQLSGLTSEDSAVYFCARKT | |
| | ISSWDFYFDYWGQGTTVTVSS | |
| 51 | DIELTQSPKFMSTSVGDRVSVTCKASQNVGTNVAWYQQKPGQSPKPLIYS | VL |
| | ATYRNSGVPDRFTGSGSGTDFTLTITNVQSKDLADYFCQQYNRYPYTSGG | |
| | GTKLEIKR | |
| 52 | GGGGSGGGGSGGGGS | Linker |
| 53 | EVKLQQSGAELVRPGSSVKISCKASGYAFSSYWMNWVKQRPGQGLEWIGQ | scFv |
| | IYPGDGDTNYNGKFKGQATLTADKSSSTAYMQLSGLTSEDSAVYFCARKT | |
| | ISSVVDFYFDYWGQGTTVTVSSGGGGSGGGGSGGGGSDIELTQSPKFMST | |
| | SVGDRVSVTCKASQNVGTNVAWYQQKPGQSPKPLIYSATYRNSGVPDRFT | |
| | GSGSGTDFTLTITNVQSKDLADYFCQQYNRYPYTSGGGTKLEIKR | |
| 54 | HYYYGGSYAMDY | HC-CDR3 |
| 55 | HTSRLHS | LC-CDR2 |
| 56 | QQGNTLPYT | LC-CDR3 |
| 57 | gacatccagatgacccagaccacetccagectgagcgccagcctgggcga | Sequence encoding |
| | ccgggtgaccatcagctgccgggccagccaggacatcagcaagtacctga | scFv |
| | actggtatcagcagaagcccgacggcaccgtcaagctgctgatctaccac | |
| | accagccggctgcacagcggcgtgcccagccggtttagcggcagcggctc | |
| | cggcaccgactacagcctgaccatctccaacctggaacaggaagatatcg | |
| | ccacctacttttgccagcagggcaacacactgccctacacctttggcggc | |
| | ggaacaaagctggaaatcaccggcagcacctccggcagcggcaagcctgg | |
| | cagcggcgagggcagcaccaagggcgaggtgaagctgcaggaaagcggcc | |
| | ctggcctggtggcccccagccagagcctgagcgtgacctgcaccgtgagc | |
| | ggcgtgagcctgcccgactacggcgtgagctggatccggcagccccccag | |
| | gaagggcctggaatggctgggcgtgatctggggcagcgagaccacctact | |
| | acaacagcgccctgaagagccggctgaccatcatcaaggacaacagcaag | |
| | agccaggtgttcctgaagatgaacagectgcagaccgacgacaccgccat | |
| | ctactactgcgccaagcactactactacggcggcagctacgccatggact | |
| | actggggccagggcaccagcgtgaccgtgagcagc | |
| 58 | X₁PPX₂P | Hinge |
| | X₁ is glycine, cysteine or arginine | |
| | X₂ is cysteine or threonine | |
| 59 | GSTSGSGKPGSGEGSTKG | Linker |

What is claimed:

1. A method of treating indolent follicular lymphoma (FL) Grade 1, 2 or 3A, the method comprising administering to a subject having or suspected of having a disease that is relapsed/refractory (r/r) follicular lymphoma (FL) Grade 1, 2 or 3A a dose of CD4⁺ and CD8⁺ T cells, wherein T cells of the dose comprises a chimeric antigen receptor (CAR) that specifically binds to CD19, wherein:
the subject has relapsed or is refractory to treatment after at least one prior line of therapy for treating FL Grade 1, 2 or 3A, wherein at least one of the at least one prior lines of therapy includes treatment with an anti-CD20 antibody and an alkylating agent;
the dose of T cells comprises between at or about 5×10⁷ CAR-expressing T cells and at or about 1.5×10⁸ CAR-expressing T cells, inclusive;
the dose of T cells comprises a ratio of approximately 1:1 CD4⁺ T cells expressing the CAR to CD8⁺ T cells expressing the CAR; and
the administration comprises administering a plurality of separate compositions, wherein the plurality of separate compositions comprises a first composition comprising the CD8⁺ CAR-expressing T cells and a second composition comprising the CD4⁺ CAR-expressing T cells.

2. The method of claim 1, wherein the at least one prior line of therapy is one prior line of therapy that includes an anti-CD20 antibody and an alkylating agent.

3. The method of claim 2, wherein the subject has relapsed or is refractory to treatment after one prior line of therapy for treating FL Grade 1, 2 or 3A, and had progression of the disease within 24 months of initiation of the one prior line of therapy that includes an anti-CD20 antibody and an alkylating agent (POD24).

4. The method of claim 2 or claim 3, wherein the subject has relapsed or is refractory to treatment after one prior line of therapy for treating FL Grade 1, 2 or 3A, and had progression of the disease within 24 months of diagnosis after completing the one prior line of therapy that includes an anti-CD20 antibody and an alkylating agent (POD24).

5. The method of any of claims 1-4, wherein the at least one prior line of therapy that includes an anti-CD20 antibody and an alkylating agent is a chemoimmunotherapeutic combination therapy that includes rituximab, cyclophosphamide, vincristine, doxorubicin, and prednisolone (R-CHOP).

6. The method of an of claims 2-5, wherein the subject received the one prior line of therapy within six months of the original FL diagnosis.

7. The method of any of claims 1-6, wherein the subject has relapsed or is refractory to treatment after one prior line of therapy for treating FL Grade 1, 2 or 3A, and has at least one of the following: symptoms attributable to FL; threatened end-organ function, cytopenia secondary to lymphoma, or bulky disease; splenomegaly; and steady progression of disease over the preceding six months or more.

8. The method of any of claims 1-7, wherein the at least one prior line of therapy is two prior lines of therapy.

9. The method of claim 8, wherein the other of the two prior lines of therapy is selected from treatment with rituximab; obinutuzumab; bendamustine plus rituximab (BR); bendamustine plus obinutuzumab (BO); R-CHOP; rituximab, cyclophosphamide, vincristine, and prednisone (R-CVP); HSCT (optionally autologous or allogeneic HSCT); lenalidomide in combination with rituximab; or a PI3K inhibitor (PI3Ki), optionally wherein the PI3Ki is idelalisib, copanlisib, or duvelisib.

10. The method of any of claims 1-9, wherein the subject is refractory to treatment or has relapsed within 12 months of completion of a prior line of therapy, optionally wherein a prior line of therapy is a combination therapy or a monotherapy comprising a PI3Ki.

11. The method of any of claims 1-10, wherein the subject has relapsed after HSCT.

12. The method of any of claims 1-11, wherein the at least one prior line of therapy is three prior lines of therapy.

13. The method of claim 12, wherein the other two of the three prior lines of therapy are each independently selected from treatment with rituximab; obinutuzumab; bendamustine plus rituximab (BR); bendamustine plus obinutuzumab (BO); R-CHOP; rituximab, cyclophosphamide, vincristine, and prednisone (R-CVP); HSCT (optionally autologous or allogeneic HSCT); lenalidomide in combination with rituximab; or a PI3K inhibitor (PI3Ki), optionally wherein the PI3Ki is idelalisib, copanlisib, or duvelisib.

14. The method of any of claims 1-13, wherein the subject is refractory to treatment with an anti-CD20 antibody.

15. The method of any of claims 1-14, wherein the subject is refractory to a prior line of therapy that includes an anti-CD20 antibody and an alkylating agent.

16. The method of any of claims 1-15, wherein the subject relapsed within six months of completing treatment with an anti-CD20 antibody.

17. The method of any of claims 1-16, wherein the subject relapsed within six months of completing a prior line of therapy that includes an anti-CD20 antibody and an alkylating agent.

18. The method of any of claims 1-17, wherein the subject has relapsed during an anti-CD20 antibody maintenance therapy following two or more lines of therapy or within six months after completion of the anti-CD20 antibody maintenance therapy.

19. The method of any of claims 1-18, wherein the subject has at least one PET-positive lesion and at least one measurable nodal lesion or extranodal lesion, optionally wherein the a measurable nodal lesion is greater than 1.5 cm in the long axis regardless of the short axis and a measurable extranodal lesion is greater than 1.0 cm in the long and short axis.

20. A method of treating marginal zone lymphoma (MZL), the method comprising administering to a subject having or suspected of having relapsed/refractory (r/r) marginal zone lymphoma (MZL) a dose of CD4⁺ and CD8⁺ T cells, wherein T cells of the dose comprises a chimeric antigen receptor (CAR) that specifically binds to CD19, wherein:
the subject has relapsed or is refractory to treatment after at least two prior lines of therapy for treating MZL;
the dose of T cells comprises between at or about 5×10⁷ CAR-expressing T cells and at or about 1.5×10⁸ CAR-expressing T cells, inclusive;
the dose of T cells comprises a ratio of approximately 1:1 CD4⁺ T cells expressing the CAR to CD8⁺ T cells expressing the CAR; and
the administration comprises administering a plurality of separate compositions, wherein the plurality of separate compositions comprises a first composition comprising the CD8⁺ CAR-expressing T cells and a second composition comprising the CD4⁺ CAR-expressing T cells.

21. The method of claim 20, wherein the MZL is a subtype selected from among extra-nodal MZL (ENMZL, mostly gastric), splenic MZL (SMZL), and nodal MZL (NMZL).

22. The method of claim 20 or claim 21, wherein at least one of the at least two prior lines of therapy is a combination systemic therapy for treating the MZL or is a hematopoietic stem cell transplant (HSCT).

23. The method of any of claims 20-22, wherein at least one of the at least two prior lines of therapy is a combination systemic therapy for treating the MZL, and the combination systemic therapy is selected from rituximab plus cyclophosphamide, doxorubicin, vincristine, and prednisone (R-CHOP), or a therapy with an anti-CD20 antibody and an alkylating agent.

24. The method of any of claims 20-23, wherein at least one of the at least two prior lines of therapy is a combination systemic therapy that is an anti-CD20 antibody and an alkylating agent.

25. The method of any of claims 20-24, wherein the subject has Splenic MZL (SMZL) and at least one of the at least two prior lines of therapy is a splenectomy.

26. The method of any of claims 20-24, wherein the subject has extranodal MZL (ENMZL), and an antibiotic is not one of the at least two prior lines of therapy.

27. The method of any of claims 20-26, wherein the subject has PET non-avid disease with at least one measurable nodal lesion greater than 2.0 cm in the long axis or at least one measurable extranodal lesion.

28. The method of any of claims 1-27, wherein the subject does not have FL grade 3B (FL3B).

29. The method of any of claims 1-28, wherein the subject does not have evidence of composite DLBCL and FL, or of transformed FL (tFL).

30. The method of any of claims 1-29, wherein the subject does not have World Health Organization (WHO) subclassification of duodenal-type FL.

31. The method of any of claims 1-30, wherein the subject has relapsed to at least one prior line of therapy, and the relapse is after an initial response of complete response (CR) or partial response (PR) to the at least one prior line of therapy.

32. The method of any of claims 1-31, wherein the subject is refractory to at least one prior line of therapy, and the refractory treatment is a best response of stable disease (SD) or progressive disease (PD) to the at least one prior line of therapy.

33. The method of any of claims 1-32, wherein the subject has an Eastern Cooperative Oncology Group (ECOG) performance status of 0 or 1.

34. The method of any of claims 1-33, wherein the anti-CD20 antibody is a monoclonal anti-CD20 antibody.

35. The method of any of claims 1-34, wherein the anti-CD20 antibody is rituximab or obinutuzumab.

36. The method of any of claims 1-35, wherein the anti-CD20 antibody is rituximab.

37. The method of any of claims 1-36, wherein the alkylating agent is bendamustine or chlorambucil.

38. The method of any of claims 1-37, further comprising administering a lymphodepleting therapy to the subject prior to administration of the dose of CD4+ and CD8+ T cells.

39. The method of claim 38, wherein the lymphodepleting therapy is completed within about 7 days prior to initiation of the administration of the dose of CD4+ and CD8+ T cells.

40. The method of claim 38 or claim 39, wherein the administration of the lymphodepleting therapy is completed within about 2 to 7 days prior to initiation of the administration of the dose of CD4+ and CD8+ T cells.

41. The method of any of claims 38-40, wherein the lymphodepleting therapy comprises the administration of fludarabine and/or cyclophosphamide.

42. The method of any of claims 38-41, wherein the lymphodepleting therapy comprises administration of cyclophosphamide at or about 200-400 mg/m², optionally at or about 300 mg/m², inclusive, and/or fludarabine at or about 20-40 mg/m², optionally 30 mg/m², daily for 2-4 days, optionally for 3 days.

43. The method of any one of claims 38-42, wherein the lymphodepleting therapy comprises administration of cyclophosphamide at or about 300 mg/m² and fludarabine at or about 30 mg/m² daily concurrently for 3 days.

44. The method of any of claims 1-43, wherein CD19 is human CD19.

45. The method of any of claims 1-44, wherein the chimeric antigen receptor (CAR) comprises an scFv comprising the variable heavy chain region and the variable light chain region of the antibody FMC63, a spacer that is 15 amino acids of less and contains an immunoglobulin hinge region or a modified version thereof, a transmembrane domain, and an intracellular signaling domain comprising a signaling domain of a CD3-zeta (CD3ζ) chain and a costimulatory signaling region that is a signaling domain of 4-1BB.

46. The method of claim 45, wherein the immunoglobulin hinge region or a modified version thereof comprises the formula X₁PPX₂P, wherein X₁ is glycine, cysteine or arginine and X₂ is cysteine or threonine (SEQ ID NO:58).

47. The method of claim 45 or claim 46, wherein the immunoglobulin hinge region or a modified version thereof is an IgG1 hinge or a modified version thereof.

48. The method of claim 45 or claim 46, wherein the immunoglobulin hinge region or a modified version thereof is an IgG4 hinge or a modified version thereof.

49. The method of any of claims 45-48, wherein the spacer comprises the sequence set forth in SEQ ID NO: 1, SEQ ID NO: 30, SEQ ID NO: 31, SEQ ID NO: 32, SEQ ID NO: 33, or SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity to the sequence set forth in SEQ ID NO: 1, SEQ ID NO: 30, SEQ ID NO: 31, SEQ ID NO: 32, SEQ ID NO: 33, or SEQ ID NO: 34.

50. The method of any of claims 45-49, wherein the spacer consists of the sequence set forth in SEQ ID NO: 1, SEQ ID NO: 30, SEQ ID NO: 31, SEQ ID NO: 32, SEQ ID NO: 33, or SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity to the sequence set forth in SEQ ID NO: 1, SEQ ID NO: 30, SEQ ID NO: 31, SEQ ID NO: 32, SEQ ID NO: 33, or SEQ ID NO: 34.

51. The method of any of claims 45-50, wherein the spacer is at or about 12 amino acids in length.

52. The method of any of claims 45-51, wherein the spacer comprises the sequence set forth in SEQ ID NO: 1.

53. The method of any of claims 45-52, wherein the spacer consists of the sequence set forth in SEQ ID NO: 1.

54. The method of any of claims 45-53, wherein the transmembrane domain is a transmembrane domain of CD28.

55. The method of any of claims 45-54, wherein the transmembrane domain comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 8.

56. The method of any of claims 45-55, wherein the costimulatory domain comprises the sequence set forth in SEQ ID NO: 12 or is a variant thereof having at least 85%, 86%, 87%, 88%, 89%, 90%, 91%, 92%, 93%, 94%, 95%, 96%, 97%, 98%, 99% or more sequence identity to the sequence set forth in SEQ ID NO: 12.

57. The method of any of claims 45-56, wherein the signaling domain of a CD3-zeta (CD3ζ) chain comprises the sequence set forth in SEQ ID NO: 13, 14, or 15, or is a variant thereof having at least 85%, 86%, 87%, 88%, 89%, 90%, 91%, 92%, 93%, 94%, 95%, 96%, 97%, 98%, 99% or more sequence identity to the sequence set forth in SEQ ID NO: 13, 14, or 15.

58. The method of any of claims 45-57, wherein the scFv comprises a CDRL1 sequence of SEQ ID NO: 35, a CDRL2 sequence of SEQ ID NO: 55, and a CDRL3 sequence of SEQ ID NO: 56; and a CDRH1 sequence of SEQ ID NO: 38, a CDRH2 sequence of SEQ ID NO: 39, and a CDRH3 sequence of SEQ ID NO: 54.

59. The method of any of claims 45-57, wherein the scFv comprises a CDRL1 sequence of RASQDISKYLN (SEQ ID NO: 35), a CDRL2 sequence of SRLHSGV (SEQ ID NO: 36), and a CDRL3 sequence of GNTLPYTFG (SEQ ID NO: 37); and a CDRH1 sequence of DYGVS (SEQ ID NO: 38), a CDRH2 sequence of VIWGSETTYYNSALKS (SEQ ID NO: 39), and a CDRH3 sequence of YAMDYWG (SEQ ID NO: 40).

60. The method of any of claims 45-59, wherein the scFv comprises, in order from N-terminus to C-terminus, a V_{L} comprising the sequence set forth in SEQ ID NO: 42, and a V_{H}, comprising the sequence set forth in SEQ ID NO: 41.

61. The method of any of claims 45-60, wherein the scFv comprises the sequence set forth in SEQ ID NO: 43.

62. The method of any of claims 1-61, wherein the CAR contains in order from N-terminus to C-terminus an extracellular antigen-binding domain that is the scFv set forth in SEQ ID NO: 43, the spacer set forth in SEQ ID NO: 1, the transmembrane domain set forth in SEQ ID NO: 8, the 4-1BB costimulatory signaling domain set forth in SEQ ID NO: 12, and the signaling domain of a CD3-zeta (CD3ζ) chain set forth in SEQ ID NO: 13.

63. The method of any of claims 1-62, wherein the dose of CD4+ and CD8+ T cells is between about 5×10⁷ CAR+ T cells and about 1.1×10⁸ CAR+ T cells, inclusive of each.

64. The method of any of claims 1-63, wherein the dose of CD4+ and CD8+ T cells is 5×10⁷CAR+ T cells.

65. The method of any of claims 1-63, wherein the dose of CD4+ and CD8+ T cells is 1×10⁸ CAR+ T cells.

66. The method of any of claims 1-65, wherein:
the first composition and the second composition are administered 0 to 12 hours apart, 0 to 6 hours apart, or 0 to 2 hours apart, or wherein the administration of the first composition and the administration of the second composition are carried out on the same day, between about 0 and about 12 hours apart, between about 0 and about 6 hours apart, or between about 0 and 2 hours apart; and/or
the initiation of administration of the first composition and the initiation of administration of the second composition are carried out between about 1 minute and about 1 hour apart or between about 5 minutes and about 30 minutes apart.

67. The method of any of claims 1-66, wherein the first composition and the second composition are administered no more than 2 hours, no more than 1 hour, no more than 30 minutes, no more than 15 minutes, no more than 10 minutes, or no more than 5 minutes apart.

68. The method of any of claims 1-67, wherein the first composition and the second composition are administered less than 2 hours apart.

69. The method of any of claims 1-68, wherein the first composition comprising the CD8⁺ CAR-expressing T cells is administered prior to the second composition comprising the CD4⁺ CAR-expressing T cells.

70. The method of any of claims 1-69, wherein the dose of CD4+ T cells and CD8+ T cells is administered intravenously.

71. The method of any of claims 1-70, wherein the T cells are primary T cells obtained from a sample from the subject, optionally wherein the sample is a whole blood sample, an apheresis sample, or a leukapheresis sample.

72. The method of claim 71, wherein the sample is obtained from the subject prior to administration of the lymphodepleting therapy to the subject.

73. The method of any of claims 1-72, wherein the T cells are autologous to the subject.

74. The method of any of claims 1-73, wherein the subject is human

75. The method of any of claims 1-74, wherein the complete response rate (CRR) among a plurality of subjects treated according to the method is greater than at or about 50%, greater than at or about 60%, greater than at or about 70%, or greater than at or about 80%.

76. The method of claim 75, wherein the CRR is the percentage of subjects with a best overall response (B OR) up to 24 months of complete response (CR).

77. The method of claim 75 or claim 76, wherein the subject has FL Grade 1, 2, or 3A, and the CRR is assessed by PET-CT.

78. The method of claim 75 or claim 76, wherein the subject has MZL, and the CRR is assessed by CT.

79. Use of a dose of CD4⁺ and CD8⁺ T cells to treat a subject having or suspected of having a disease that is relapsed/refractory (r/r) follicular lymphoma (FL) Grade 1, 2 or 3A, wherein:
T cells of the dose comprise a chimeric antigen receptor (CAR) that specifically binds to CD19;
the subject has relapsed or is refractory to treatment after at least one prior line of therapy for treating FL Grade 1, 2 or 3A, wherein the at least one of the at least one prior line of therapy includes treatment with an anti-CD20 antibody and an alkylating agent;
the dose of T cells comprises between at or about 5×10⁷ CAR-expressing T cells and at or about 1.5×10⁸ CAR-expressing T cells, inclusive;
the dose of T cells comprises a ratio of approximately 1:1 CD4⁺ T cells expressing the CAR to CD8⁺ T cells expressing the CAR; and
the dose is formulated for administration as a plurality of separate compositions, wherein the plurality of separate compositions comprises a first composition comprising the CD8⁺ CAR-expressing T cells and a second composition comprising the CD4⁺ CAR-expressing T cells.

80. Use of a dose of CD4⁺ and CD8⁺ T cells in the manufacture of a medicament for the treatment of a subject having or suspected of having a disease that is relapsed/refractory (r/r) follicular lymphoma (FL) Grade 1, 2 or 3A, wherein:
T cells of the dose comprise a chimeric antigen receptor (CAR) that specifically binds to CD19;
the subject has relapsed or is refractory to treatment after at least one prior line of therapy for treating FL Grade 1, 2 or 3A, wherein at least one of the at least one prior line of therapy includes treatment with an anti-CD20 antibody and an alkylating agent;
the dose of T cells comprises between at or about 5×10⁷ CAR-expressing T cells and at or about 1.5×10⁸ CAR-expressing T cells, inclusive;
the dose of T cells comprises a ratio of approximately 1:1 CD4⁺ T cells expressing the CAR to CD8⁺ T cells expressing the CAR; and
the dose is formulated for administration as a plurality of separate compositions, wherein the plurality of separate compositions comprises a first composition comprising the CD8⁺ CAR-expressing T cells and a second composition comprising the CD4⁺ CAR-expressing T cells.

81. Use of a dose of CD4⁺ and CD8⁺ T cells to treat a subject having or suspected of having relapsed/refractory (r/r) marginal zone lymphoma (MZL), wherein:
T cells of the dose comprises a chimeric antigen receptor (CAR) that specifically binds to CD19;
the subject has relapsed or is refractory to treatment after at least two prior lines of therapy for treating MZL;
the dose of T cells comprises between at or about 5×10⁷ CAR-expressing T cells and at or about 1.5×10⁸ CAR-expressing T cells, inclusive;
the dose of T cells comprises a ratio of approximately 1:1 CD4⁺ T cells expressing the CAR to CD8⁺ T cells expressing the CAR; and
the dose is formulated for administration of a plurality of separate compositions, wherein the plurality of separate compositions comprises a first composition comprising the CD8⁺ CAR-expressing T cells and a second composition comprising the CD4⁺ CAR-expressing T cells.

82. Use of a dose of CD4⁺ and CD8⁺ T cells in the manufacture of a medicament for treatment of a subject having or suspected of having relapsed/refractory (r/r) marginal zone lymphoma (MZL), wherein:
T cells of the dose comprises a chimeric antigen receptor (CAR) that specifically binds to CD19;
the subject has relapsed or is refractory to treatment after at least two prior lines of therapy for treating MZL;
the dose of T cells comprises between at or about 5×10⁷ CAR-expressing T cells and at or about 1.5×10⁸ CAR-expressing T cells, inclusive;
the dose of T cells comprises a ratio of approximately 1:1 CD4⁺ T cells expressing the CAR to CD8⁺ T cells expressing the CAR; and
the dose is formulated for administration of a plurality of separate compositions, wherein the plurality of separate compositions comprises a first composition comprising the CD8⁺ CAR-expressing T cells and a second composition comprising the CD4⁺ CAR-expressing T cells.
